SEQUENCE LISTING (25 sequences)

SEQ ID NO: 1 - protein, 8 aa, synthetic construct
DYKDDDDK

SEQ ID NO: 2 - protein, 5 aa, synthetic construct
DQNAT

SEQ ID NO: 3 - protein, 659 aa, Escherichia coli
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 4 - DNA, 1977 bp, Escherichia coli
  source 1..1977: mol_type = other DNA
atgatcacccacggttgctacacccgtacccgtcacaagcacaaactgaagaaaaccctgattatgctgagcgcgggtctgggcctgttcttttacgttaaccagaacagcttcgcgaacggcgagaactattttaagctgggcagcgacagcaaactgctgacccacgatagctaccagaaccgtctgttctataccctgaaaaccggtgaaaccgtggcggacctgagcaagagccaagatatcaacctgagcaccatttggagcctgaacaaacacctgtacagcagcgagagcgaaatgatgaaggcggcgccgggccagcaaatcattctgccgctgaagaaactgccgtttgagtatagcgcgctgccgctgctgggtagcgcgccgctggttgcggcgggtggcgtggcgggccacaccaacaagctgaccaaaatgagcccggacgttaccaaaagcaacatgaccgacgataaagcgctgaactatgcggcgcagcaagcggcgagcctgggtagccagctgcaaagccgtagcctgaacggcgactatgcgaaagataccgcgctgggtatcgcgggcaaccaagcgagcagccagctgcaagcgtggctgcagcactacggcaccgcggaagtgaacctgcaaagcggtaacaacttcgacggcagcagcctggatttcctgctgccgttttacgacagcgaaaaaatgctggcgtttggtcaagtgggtgcgcgttatattgatagccgttttaccgcgaacctgggtgcgggccagcgtttctttctgccggcgaacatgctgggttacaacgttttcatcgaccaagattttagcggtgacaacacccgtctgggcattggtggcgaatactggcgtgattatttcaaaagcagcgtgaacggttattttcgtatgagcggctggcacgagagctacaacaagaaagactatgatgaacgtccggcgaacggtttcgacatccgttttaacggctacctgccgagctatccggcgctgggtgcgaaactgatttacgagcagtactatggcgacaacgttgcgctgttcaacagcgataagctgcaaagcaacccgggtgcggcgaccgttggcgtgaactacaccccgatcccgctggtgacgatgggtattgactatcgtcacggcaccggcaacgaaaacgatctgctgtactccatgcagttccgttatcagtttgacaagagctggagccagcaaatcgagccgcagtacgttaacgaactgcgtaccctgagcggcagccgttatgatctggtgcagcgtaacaacaacatcattctggagtacaagaaacaagacattctgagcctgaacatcccgcacgatattaacggcaccgaacacagcacccagaagatccaactgatcgttaagagcaagtacggcctggaccgtatcgtgtgggacgatagcgcgctgcgtagccagggtggccagattcaacacagcggtagccagagcgcgcaagattaccaggcgatcctgccggcgtatgttcaaggtggcagcaacatttacaaggtgaccgcgcgtgcgtatgaccgtaacggcaacagcagcaacaacgttcagctgaccatcaccgtgctgagcaacggtcaagtggttgatcaggttggcgtgaccgacttcaccgcggataagaccagcgcgaaagcggacaacgcggataccatcacctacaccgcgaccgttaagaaaaacggtgtggcgcaggcgaacgttccggtgagctttaacattgtgagcggcaccgcgaccctgggtgcgaacagcgcgaagaccgacgcgaacggtaaagcgaccgtgaccctgaagagcagcaccccgggtcaagtggttgtgagcgcgaaaaccgcggagatgaccagcgcgctgaacgcgagcgcggtgatcttctttgatcagaccaaggcgagc

SEQ ID NO: 5 - protein, 303 aa, Escherichia coli
MTEIVADKTVEVVKNAIETADGALDLYNKYLDQVIPWQTFDETIKELSRFKQEYSQAASVLVGDIKTLLMDSQDKYFEATQTVYEWCGVATQLLAAYILLFDEYNEKKASAQKDILIKVLDDGITKLNEAQKSLLVSSQSFNNASGKLLALDSQLTNDFSEKSSYFQSQVDKIRREAYAGAAAGVVAGPFGLIISYSIAAAVVEGKLIPELKNKLKSVQNFFTTLSNTVKQANKDIDAAKLKLTTEIAAIGEIKTETETTRFYVDYDDLMLSLLKEAAKKMINTCNEYQKRHGKKTLFEVPEV

SEQ ID NO: 6 - DNA, 909 bp, Escherichia coli
  source 1..909: mol_type = other DNA
atgaccgagattgtggcggacaaaaccgttgaggtggttaagaacgcgatcgaaaccgcggacggtgcgctggatctgtacaacaaatatctggaccaagtgattccgtggcaaaccttcgatgaaaccatcaaagaactgagccgttttaagcaggaatacagccaagcggcgagcgtgctggttggtgatattaaaaccctgctgatggacagccaggataagtacttcgaggcgacccaaaccgtgtatgaatggtgcggtgttgcgacccagctgctggcggcgtacattctgctgtttgacgagtataacgaaaagaaagcgagcgcgcaaaaagatatcctgattaaggtgctggacgatggtatcaccaaactgaacgaggcgcagaagagcctgctggttagcagccaaagcttcaacaacgcgagcggcaagctgctggcgctggacagccagctgaccaacgatttcagcgagaaaagcagctactttcagagccaagtggacaagatccgtcgtgaagcgtatgcgggtgcggcggcgggcgtggttgcgggtccgtttggcctgatcattagctacagcattgcggcggcggtggttgagggcaaactgatcccggaactgaagaacaaactgaagagcgtgcagaacttctttaccaccctgagcaacaccgttaaacaagcgaacaaggacattgatgcggcgaaactgaagctgaccaccgagatcgcggcgattggtgaaatcaagaccgaaaccgaaaccacccgtttctacgttgattatgacgatctgatgctgagcctgctgaaagaggcggcgaagaaaatgatcaacacctgcaacgaatatcagaagcgtcacggcaagaaaaccctgtttgaggtgccggaagtt

SEQ ID NO: 7 - protein, 145 aa, Escherichia coli
MKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 8 - DNA, 435 bp, Escherichia coli
  source 1..435: mol_type = other DNA
atgaaggcgaccaaactggtgctgggtgcggttattctgggcagcaccctgctggcgggttgcagcagcaacgcgaaaatcgaccagggcattaacccgtacgtgggtttcgaaatgggctatgattggctgggtcgtatgccgtacaagggtagcgtggagaacggcgcgtataaagcgcagggtgttcaactgaccgcgaagctgggctacccgatcaccgacgatctggacatttatacccgtctgggtggcatggtgtggcgtgcggacaccaagagcaacgtttacggtaaaaaccacgataccggcgtgagcccggtttttgcgggtggcgttgagtacgcgatcaccccggaaattgcgacccgtctggagtatcaatggaccaacaacatcggtgacgcgcacaccattggcacccgtccggataac

SEQ ID NO: 9 - protein, 287 aa, Escherichia coli
SYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 10 - DNA, 861 bp, Escherichia coli
  source 1..861: mol_type = other DNA
agctataacaactttatcaccgaggtgggcagcctgaacaagcgtatgggtgacctgcgtgatattaacggcgaagcgggcacctgggttcgtctgctgaacggcagcggtagcgcggatggtggctttaccgaccactacaccctgctgcagatgggcgcggatcgtaaacacgagctgggcagcatggacctgttcaccggtgtgatggcgacctataccgacaccgatgcgagcgcggatctgtacagcggcaagaccaaaagctggggtggcggtttctatgcgagcggcctgtttcgtagcggtgcgtacttcgatgtgatcgcgaagtatattcacaacgagaacaaatacgacctgaactttgcgggcgcgggcaagcagaactttcgtagccacagcctgtatgcgggtgcggaagttggttaccgttatcacctgaccgacaccacctttgtggagccgcaagcggaactggtttggggccgtctgcagggtcaaaccttcaactggaacgatagcggcatggacgtgtccatgcgtcgtaacagcgtgaacccgctggttggccgtaccggtgtggttagcggcaagacctttagcggtaaagattggagcctgaccgcgcgtgcgggtctgcactatgagttcgacctgaccgatagcgcggacgttcacctgaaggatgcggcgggcgaacaccaaatcaacggtcgtaaagacagccgtatgctgtacggcgtgggtctgaacgcgcgttttggcgacaacacccgtctgggtctggaagtggaacgtagcgcgttcggtaaatataacaccgacgatgcgatcaacgcgaacattcgttacagcttc

SEQ ID NO: 11 - protein, 340 aa, Escherichia coli
LRSENAYRAEVPLYASMLTQAMDYDRILAGSRSHQTGVNGENNSVRLSIQGGHLGHDNNGGIVRGATPESSGSYGFVRLEGDLLRTEVAGMSLTTGVYGAAGHSSVDVKDDDGSRAGTVRDDAGSLGGYLNLVHTSSGLWADIVAQGTRHSMKASSDNNDFRARGRGWQGSLETGLPFSITDNLMLEPQLQYTWQGLSLDDGQDNAGYVKFGHGSAQHVRAGFRLGSHNDMTFGEGTSSRDTLRDSTKHGVSELPVNWWVQPSVIRTFSSRGDMSMGTAAAGSNMTFSPSRNGTSLDLQAGLEARVRENITLGVQAGYAHSVSGNSAEGYNGQATLNVTF

SEQ ID NO: 12 - DNA, 1020 bp, Escherichia coli
  source 1..1020: mol_type = other DNA
ctgcgtagcgagaacgcgtaccgtgcggaagtgccgctgtatgcgtccatgctgacccaggcgatggattacgaccgtatcctggcgggtagccgtagccaccaaaccggtgttaacggcgagaacaacagcgtgcgtctgagcatccagggtggccacctgggtcacgataacaacggtggcattgttcgtggcgcgaccccggaaagcagcggtagctacggcttcgttcgtctggagggtgacctgctgcgtaccgaagtggcgggcatgagcctgaccaccggtgtttatggcgcggcgggtcacagcagcgtggatgttaaagatgatgatggcagccgtgcgggcaccgttcgtgatgatgcgggtagcctgggtggctatctgaacctggtgcacaccagcagcggtctgtgggcggacatcgttgcgcaaggcacccgtcacagcatgaaagcgagcagcgataacaacgattttcgtgcgcgtggtcgtggctggcagggtagcctggaaaccggtctgccgtttagcattaccgataacctgatgctggaaccgcagctgcaatacacctggcagggtctgagcctggatgacggccaagacaacgcgggttatgtgaagttcggtcacggcagcgcgcaacatgttcgtgcgggtttccgtctgggtagccacaacgatatgacctttggcgagggcaccagcagccgtgataccctgcgtgacagcaccaaacacggcgtgagcgaactgccggtgaactggtgggttcagccgagcgtgatccgtaccttcagcagccgtggcgatatgagcatgggcaccgcggcggcgggcagcaacatgacctttagcccgagccgtaacggcaccagcctggacctgcaagcgggcctggaggcgcgtgttcgtgaaaacattaccctgggcgtgcaggcgggttatgcgcacagcgttagcggtaacagcgcggagggctataacggtcaagcgaccctgaacgttaccttt

SEQ ID NO: 13 - protein, 288 aa, Neisseria gonorrhoeae
STNTNSALSDAMASTQSILLDTGASLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGAALLYRNVTLNVNGSITKGKQLEKQKSGQIKIQIRF

SEQ ID NO: 14 - DNA, 864 bp, Neisseria gonorrhoeae
  source 1..864: mol_type = other DNA
agcaccaacaccaacagcgcgctgagcgatgcgatggcgagcacccagagcattctgctggataccggtgcgagcctgacccgtcacattgcgcaaaagagccgtgcggacgcggagaaaaacagcgtgtggatgagcaacaccggttacggccgtgattatgcgagcgcgcagtatcgtcgtttcagcagcaaacgtacccaaacccagatcggcattgaccgtagcctgagcgaaaacatgcaaattggtggcgttctgacctacagcgacagccaacacaccttcgatcaggcgggtggcaaaaacacctttgtgcaggcgaacctgtatggcaagtactatctgaacgacgcgtggtacgtggcgggcgatattggtgcgggcagcctgcgtagccgtctgcaaacccagcaaaaagcgaacttcaaccgtaccagcatccagaccggtctgaccctgggcaacaccctgaagattaaccaatttgagatcgtgccgagcgcgggtatccgttacagccgtctgagcagcgcggactataagctgggcgacgatagcgtgaaagttagcagcatggcggttaagaccctgaccgcgggtctggatttcgcgtaccgttttaaagtgggcaacctgaccgttaagccgctgctgagcgcggcgtacttcgcgaactatggtaaaggtggcgtgaacgttggtggcaagagctttgcgtacaaagcggataaccagcaacaatacagcgcgggtgcggcgctgctgtaccgtaacgtgaccctgaacgttaacggtagcattaccaagggcaaacaactggaaaagcagaaaagcggtcaaatcaagattcagatccgtttt

SEQ ID NO: 15 - protein, 325 aa, Neisseria gonorrhoeae
QYRPENGSYATNMALANSLFLMDLNERKQFRAMSDNTQPESASVWMKITGGISSGKLNDGQNKTTTNQFINQLGGDIYKFHAEQLGDFTLGIMGGYANAKGKTINYTSNKAARNTLDGYSVGVYGTWYQNGENATGLFAETWMQYNWFNASVKGDGLEEEKYNLNGLTASAGGGYNLNVHTWTSPEGITGEFWLQPHLQAVWMGVTPDTHQEDNGTVVQGAGKNNIQTKAGIRASWKVKSTLDKDTGRRFRPYIEANWIHNTHEFGVKMSDDSQLLSGSRNQGEIKTGIEGVITQNLSVNGGVAYQAGGHGSNAISGALGIKYSF

SEQ ID NO: 16 - DNA, 975 bp, Neisseria gonorrhoeae
  source 1..975: mol_type = other DNA
cagtaccgtccggaaaacggttcttatgcaactaacatggcgctggcgaacagcctgttcctgatggatctgaacgaacgtaaacaattccgcgctatgagcgacaatactcagccagaatctgcgagcgtttggatgaaaattaccggtggtatctctagcggcaagctgaacgatggtcagaacaaaactaccaccaaccaattcattaaccagctgggcggtgacatttacaagttccacgctgaacagctgggtgactttacgctgggcatcatgggtggttacgccaacgcgaaaggcaaaactatcaactacactagcaacaaagcagcgcgcaatacgctggacggttactctgtgggcgtttacggcacttggtatcagaatggtgaaaacgccacgggcctgttcgcggaaacctggatgcagtacaactggttcaacgcgtctgtgaaaggcgacggtctggaagaggaaaagtacaacctgaacggtctgactgcaagcgctggcggcggttacaatctgaacgtccatacttggaccagcccggaaggtatcaccggcgaattttggctgcaaccgcacctgcaggctgtctggatgggcgttaccccggacacccaccaagaagataatggcaccgttgtgcagggcgcaggcaaaaacaatatccagactaaagccggtatccgcgcgtcctggaaagtgaaatctacgctggataaagacaccggccgtcgcttccgtccgtacatcgaagcgaattggattcacaacactcacgagttcggcgtgaagatgtctgatgactctcagctgctgtccggcagccgtaaccaaggcgaaatcaaaaccggcatcgagggtgtaatcacccagaacctgtctgttaacggtggcgttgcgtatcaggcaggtggtcatggctccaacgcgatcagcggcgctctgggcatcaaatactctttt

SEQ ID NO: 17 - protein, 5 aa, synthetic construct
GGGGS

SEQ ID NO: 18 - protein, 10 aa, synthetic construct
GGGGSGGGGS

SEQ ID NO: 19 - protein, 15 aa, synthetic construct
GGGGSGGGGSGGGGS

SEQ ID NO: 20 - protein, 20 aa, synthetic construct
GGGGSGGGGSGGGGSGGGGS

SEQ ID NO: 21 - protein, 6 aa, synthetic construct
GGGGGG

SEQ ID NO: 22 - protein, 8 aa, synthetic construct
GGGGGGGG

SEQ ID NO: 23 - protein, 12 aa, synthetic construct
GSAGSAAGSGEF

SEQ ID NO: 24 - protein, 10 aa, synthetic construct
EQKLISEEDL

SEQ ID NO: 25 - protein, 5 aa, synthetic construct
DDDDK

(19) United States
(12) Patent Application Publication
(10) Pub. No.: US 2023/0083394 A1
(43) Pub. Date: Mar. 16, 2023

(54) METHODS AND COMPOSITIONS FOR DOCKING BIOTINYLATED ANTIGENS ON THE EXTERIOR OF BACTERIAL OUTER MEMBRANE VESICLES

(71) Applicant: CORNELL UNIVERSITY, Ithaca, NY (US)

(72) Inventors: Matthew P. DELISA, Ithaca, NY (US); Kevin WEYANT, Ithaca, NY (US)

(21) Appl. No.: 17/895,245

(22) Filed: Aug. 25, 2022

Related U.S. Application Data
(60) Provisional application No. 63/237,075, filed on Aug. 25, 2021.

(51) Int. Cl. C12N 15/10; C07K 14/005; C07K 16/44; A61K 39/385; A61K 47/54; A61K 47/61; C07K 14/195

(52) U.S. Cl. CPC C12N 15/1037; C07K 14/005; C07K 16/44; A61K 39/385; A61K 47/557; A61K 47/543; A61K 47/61; C07K 14/195

(57) ABSTRACT

The present disclosure is directed to a system for displaying antigens. This system includes an outer membrane vesicle comprising a lipid bilayer and a synthetic antigen receptor comprising an outer membrane scaffold protein fused to a biotin-binding protein, where the outer membrane scaffold protein is incorporated in the lipid bilayer and the biotin-binding protein is displayed outside the outer membrane vesicle. Also disclosed are therapeutic compositions, nucleic acid constructs, expression vectors, and methods of eliciting an immune response in a subject.

This application claims the benefit of U.S. Provisional Patent Application Ser. No. 63/237,075, filed Aug. 25, 2021, which is hereby incorporated by reference in its entirety.

This invention was made with government support under grant HDTRA1-20-10004 awarded by Defense Threat Reduction Agency, grant R01GM137314 and grant R01GM127578 awarded by National Institutes of Health, and grant CBET-1605242 and grant CBET-1936823 awarded by National Science Foundation. The government has certain rights in the invention.

### FIELD

The present disclosure is directed to a system for displaying antigen comprising an outer membrane vesicle (OMV) and synthetic antigen receptor (SNARE) proteins comprising a scaffold protein fused to a biotin-binding protein. Also disclosed are therapeutic compositions, nucleic acid constructs, expression vectors, and methods of eliciting an immune response in a subject.

### SEQUENCE LISTING

The instant application contains a Sequence Listing which has been submitted in XML format and is hereby incorporated by reference in its entirety. Said XML copy, created on Oct. 9, 2022, is named 147402_009052.xml and is 32 kilobytes in size.

### BACKGROUND

Outer membrane vesicles (OMVs) are spherical bilayered nanostructures (˜20-250 nm) ubiquitously released from the cell envelope of Gram-negative and Gram-positive bacteria and their production represents a bona fide bacterial secretion process(Schwechheimer and Kuehn, “Outer-Membrane Vesicles from Gram-Negative Bacteria: Biogenesis and Functions,” Nat. Rev. Microbiol. 13:605-619 (2015); Kulp and Kuehn, “Biological Functions and Biogenesis of Secreted Bacterial Outer Membrane Vesicles,” Annual Review of Microbiology 64:163-184 (2010)). As derivatives of the cell envelope, OMVs mimic the structural organization and conformation of the bacterial cell surface while also containing periplasmic lumenal components. Natively produced OMVs mediate diverse functions such as increasing pathogenicity in the host environment (Vidakovics et al., “B Cell Activation by Outer Membrane Vesicles—A Novel Virulence Mechanism,” PLoS Pathog. 6:e1000724 (2010)), promoting bacterial survival under conditions of stress (McBroom and Kuehn, “Release of Outer Membrane Vesicles by Gram-Negative Bacteria is a Novel Envelope Stress Response,” Mol. Microbiol. 63:545-558 (2007)), and controlling interactions within microbial communities (Biller et al., “Bacterial Vesicles in Marine Ecosystems,” Science 343:183-186 (2014)).

In addition to their natural biological roles, OMVs have enabled a spectrum of bioengineering applications, most notably in drug and vaccine delivery, that exploit the unique structural and functional attributes of these nanoparticle systems (Gnopo et al., “Designer Outer Membrane Vesicles as Immunomodulatory Systems—Reprogramming Bacteria for Vaccine Delivery,” Adv. Drug Deliv. Rev. 114:132-142 (2017); Rosenthal et al., “Pathogen-Like Particles: Biomimetic Vaccine Carriers Engineered at the Nanoscale,” Curr. Opin. Biotechnol. 28:51-58 (2014); Jahromi and Fuhrmann, “Bacterial Extracellular Vesicles: Understanding Biology Promotes Applications as Nanopharmaceuticals,” Adv. Drug Deliv. Rev 173:125-140 (2021); and Li et al., “Bacterial Outer Membrane Vesicles as a Platform for Biomedical Applications: An Update,” J. Control Release 323:253-268 (2020)). OMVs are especially attractive as a vaccine platform because they are non-replicating, immunogenic facsimiles of the producing bacteria and thus contain the pathogen-associated molecular patterns (PAMPs) present on bacterial outer membranes (Gnopo et al., “Designer Outer Membrane Vesicles as Immunomodulatory Systems—Reprogramming Bacteria for Vaccine Delivery,” Adv. Drug Deliv. Rev. 114:132-142 (2017) and Rosenthal et al., “Pathogen-Like Particles: Biomimetic Vaccine Carriers Engineered at the Nanoscale,” Curr. Opin. Biotechnol. 28:51-58 (2014)). These PAMPs endow OMVs with intrinsic immunostimulatory properties that strongly stimulate innate and adaptive immune responses (Alaniz et al., “Membrane Vesicles are Immunogenic Facsimiles of Salmonella typhimurium that Potently Activate Dendritic Cells, Prime B and T Cell Responses, and Stimulate Protective Immunity in vivo,” J. Immunol. 179:7692-7701 (2007); Sanders and Feavers, “Adjuvant Properties of Meningococcal Outer Membrane Vesicles and the use of Adjuvants in Neisseria meningitidis Protein Vaccines,” Expert. Rev. Vaccines 10:323-334 (2011); Ellis et al., “Naturally Produced Outer Membrane Vesicles from Pseudomonas aeruginosa Elicit a Potent Innate Immune Response via Combined Sensing of Both Lipopolysaccharide and Protein Components,” Infect. Immun. 78:3822-3831 (2010); Kaparakis-Liaskos and Ferrero, “Immune Modulation by Bacterial Outer Membrane Vesicles,” Nat. Rev. Immunol. 15:375-387 (2015)). In addition to this in-built adjuvanticity, OMVs are right-sized for direct drainage into lymph nodes and subsequent uptake by antigen presenting cells and cross-presentation (Bachmann and Jennings, “Vaccine Delivery: A Matter of Size, Geometry, Kinetics and Molecular Patterns,” Nat. Rev. Immunol. 10:787-796 (2010)). From a translational perspective, OMVs can be readily produced at high quantities and commercial scales via standard bacterial fermentation, and their clinical use has already been established in the context of OMVs from pathogenic Neisseria meningitidis serogroup B (MenB), also known as outer membrane protein complexes (OMPCs), that are the basis of a polyribosylribitol phosphate (PRP) conjugate vaccine approved for Haemophilus influenzae type b called PedvaxHlB® (Vella et al., “Immunogenicity of a New Haemophilus influenzae Type B Conjugate Vaccine (Meningococcal Protein Conjugate) (PedvaxHIB),” Pediatrics 85:668-675 (1990)) and are a component of the MenB vaccine Bexsero® (Giuliani et al., “A Universal Vaccine for Serogroup B meningococcus,” Proc. Natl. Acad. Sci. USA 103:10834-10839 (2006).

To generalize and expand the vaccine potential of OMVs, recombinant DNA technology and synthetic biology techniques have been leveraged to engineer OMVs with heterologous protein and peptide cargo (Kesty and Kuehn, “Incorporation of Heterologous Outer Membrane and Periplasmic Proteins into Escherichia coli Outer Membrane Vesicles,” J. Biol. Chem. 279:2069-2076 (2004); Kim et al., “Engineered Bacterial Outer Membrane Vesicles with Enhanced Functionality,” J. Mol. Biol. 380:51-66 (2008)). By targeting expression to the outer membrane or the periplasm of an OMV-producing host strain, both surface display as well as payload encapsulation are possible, providing versatility as biomedical research tools and vaccines. Typically, this involves genetic fusion of a protein or peptide of interest (POI) to an outer membrane scaffold protein (e.g., the E. coli cytolysin ClyA), with the resulting POI accumulating in released OMVs that can be readily recovered from the culture supernatant. These methods have made it possible to enlist non-pathogenic, genetically tractable bacteria such as Escherichia coli K-12 for the production of designer OMVs that are loaded with foreign antigens of interest (Gnopo et al., “Designer Outer Membrane Vesicles as Immunomodulatory Systems—Reprogramming Bacteria for Vaccine Delivery,” Adv. Drug Deliv. Rev. 114:132-142 (2017) and Baker et al., “Microbial Biosynthesis of Designer Outer Membrane Vesicles,” Curr. Opin. Biotechnol. 29:76-84 (2014)). When inoculated in mice, such engineered OMVs stimulate antigen-specific humoral B cell and dendritic cell (DC)-mediated T cell responses including activation of CD4⁺ and CD8⁺ T cells (Alaniz et al., “Membrane Vesicles are Immunogenic Facsimiles of Salmonella typhimurium that Potently Activate Dendritic Cells, Prime B and T Cell Responses, and Stimulate Protective Immunity in vivo,” J. Immunol. 179:7692-7701 (2007); Schetters et al., “Outer Membrane Vesicles Engineered to Express Membrane-Bound Antigen Program Dendritic Cells for Cross-Presentation to CD8(+) T Cells,” Acta Biomater. 91:248-257 (2019); Rosenthal et al., “Mechanistic Insight into the TH1-Biased Immune Response to Recombinant Subunit Vaccines Delivered by Probiotic Bacteria-Derived Outer Membrane Vesicles,” PLoS One 9:e112802 (2014); and Chen et al., “Delivery of Foreign Antigens by Engineered Outer Membrane Vesicle Vaccines,” Proc. Natl. Acad. Sci. USA 107:3099-3104 (2010)). Importantly, the immune responses triggered by antigen-loaded OMV vaccines have proven to be protective against a range of foreign pathogens including bacteria and viruses (Muralinath et al., “Immunization with Salmonella Enterica Serovar Typhimurium-Derived Outer Membrane Vesicles Delivering the pneumococcal Protein PspA Confers Protection Against Challenge with Streptococcus Pneumoniae,” Infect. Immun. 79:887-894 (2011); Fantappie et al., “Antibody-Mediated Immunity Induced by Engineered Escherichia coli OMVs Carrying Heterologous Antigens in their Lumen,” J. Extracell. Vesicles 3 (2014); Rappazzo et al., Recombinant M2e Outer Membrane Vesicle Vaccines Protect Against Lethal Influenza A Challenge in BALB/c Mice,” Vaccine 34:1252-1258 (2016); and Bartolini et al., “Recombinant Outer Membrane Vesicles Carrying Chlamydia Muridarum HtrA Induce Antibodies that Neutralize Chlamydial Infection in Vitro,” J. Extracell. Vesicles 2 (2013)) as well as against malignant tumors (Grandi et al., “Synergistic Protective Activity of Tumor-Specific Epitopes Engineered in Bacterial Outer Membrane Vesicles,” Front. Oncol. 7:253 (2017)). While proteins and peptides remain the focus of most OMV-based vaccine efforts, advances in bacterial glycoengineering have enabled decoration of OMV exteriors with heterologous polysaccharide antigens, giving rise to a new class of glycoconjugate vaccines that can effectively deliver pathogen-mimetic glycan epitopes to the immune system and confer protection to subsequent pathogen challenge (Chen et al., “Outer Membrane Vesicles Displaying Engineered Glycotopes Elicit Protective Antibodies,” Proc. Natl. Acad. Sci. USA 113:E3609-3618 (2016); Valentine et al., “Immunization with Outer Membrane Vesicles Displaying Designer Glycotopes Yields Class-Switched, Glycan-Specific Antibodies,” Cell Chem. Biol. 23:655-665 (2016); and Stevenson et al., “Immunization with Outer Membrane Vesicles Displaying Conserved Surface Polysaccharide Antigen Elicits Broadly Antimicrobial Antibodies,” Proc. Natl. Acad. Sci. USA 115:E3106-E3115 (2018)). Collectively, these and other studies have revealed that the repetitive, high-density arrangement of antigens on the OMV surface enhances the response to otherwise poorly immunogenic epitopes such as small peptides and polysaccharides, which likely results from induction of strong B-cell receptor clustering.

These successes notwithstanding, the classical approach to loading OMVs with foreign antigens prior to their isolation from bacterial cultures is not without its challenges. For example, many antigens that are desirable from a vaccine standpoint are incompatible with recombinant expression in the lumen or on the surface of OMVs. While there can be many reasons for this, the most common bottlenecks include misfolding, proteolytic degradation, and/or inefficient bilayer translocation of the POI, especially for those that are very bulky and/or structurally complex. Because there are currently no effective tools for predicting a priori the expressibility of OMV-directed antigens, the creation of heterologous OMV vaccines remains very much a time-consuming trial-and-error process that often must be repeated for each new antigen. Even when a foreign antigen can be successfully localized to OMVs, it may lack important post-translational modifications that are formed inefficiently (or not at all) in the bacterial expression host. In addition, it can be difficult or even impossible to precisely control the quantity of OMV-associated antigen, thereby excluding antigen density as a customizable design parameter. It should also be noted that while it is possible to integrate polypeptide and polysaccharide biosynthesis with the vesiculation process (Gnopo et al., “Designer Outer Membrane Vesicles as Immunomodulatory Systems—Reprogramming Bacteria for Vaccine Delivery,” Adv. Drug Deliv. Rev. 114:132-142 (2017); Baker et al., “Microbial Biosynthesis of Designer Outer Membrane Vesicles,” Curr. Opin. Biotechnol. 29:76-84 (2014)), it has yet to be demonstrated whether biosynthesis of other biomolecules can be similarly integrated, thereby limiting the spectrum of cargo that can be packaged in OMVs.

To address these shortcomings, reliable strategies are needed for modular OMV functionalization in which OMV vectors and structurally diverse target antigens are separately produced and then subsequently linked together in a controllable fashion. Along these lines, direct chemical conjugation of proteins and polysaccharides to OMVs/OMPCs following their isolation has been reported (Vella et al., “Immunogenicity of a New Haemophilus influenzae Type B Conjugate Vaccine (Meningococcal Protein Conjugate) (PedvaxHIB),” Pediatrics 85:668-675 (1990); Wu et al., “Sustained High-Titer Antibody Responses Induced by Conjugating a Malarial Vaccine Candidate to Outer-Membrane Protein Complex,” Proc. Natl. Acad. Sci. USA 103:18243-18248 (2006); however, this technique involves non-specific attachment of antigens to unknown OMV components and thus is heterogeneous and difficult to predict or analyze. Moreover, non-uniform coupling of antigen to particulate carriers may result in sub-optimal immunogenicity. For more precise, homogenous antigen attachment, site-specific conjugation methods are preferrable. To this end, two groups recently demonstrated specific bioconjugation on OMVs by adapting a “plug-and-display” approach that had previously been developed for decorating virus-like particles with protein and peptide antigens (Brune et al., “Plug-and-Display: Decoration of Virus-Like Particles Via Isopeptide Bonds for Modular Immunization,” Sci. Rep. 6:19234 (2016)). This approach involved the use of the SpyTag/SpyCatcher protein ligation system to covalently attach purified SpyTag-antigen (or SpyCatcher-antigen) fusion proteins onto cognate SpyCatcher-scaffold (or SpyTag-scaffold) fusions that were expressed on the surface of OMVs (Cheng et al., “Bioengineered Bacteria-Derived Outer Membrane Vesicles as a Versatile antigen Display Platform for Tumor Vaccination via Plug-and-Display Technology,” Nat. Commun. 12:2041 (2021); van den Berg et al., “Display of Recombinant Proteins on Bacterial Outer Membrane Vesicles by Using Protein Ligation,” Appl. Environ. Microbiol. 84(8):e02567-17 (2018)). While this enabled loading of exogenous antigens on OMVs, with one report even demonstrating specific anti-tumor immune responses (Cheng et al., “Bioengineered Bacteria-Derived Outer Membrane Vesicles as a Versatile antigen Display Platform for Tumor Vaccination via Plug-and-Display Technology,” Nat. Commun. 12:2041 (2021)), the protein ligation strategy is limited to proteinaceous antigens that are compatible with isopeptide bond formation. A more universal strategy is needed for tethering virtually any biomolecular cargo to the exterior of OMVs.

The present application is directed to overcoming these and other deficiencies in the art.

### SUMMARY

One aspect of the present disclosure relates to a system for displaying antigens. This system includes an outer membrane vesicle comprising a lipid bilayer and a synthetic antigen receptor comprising an outer membrane scaffold protein fused to a biotin-binding protein, where the outer membrane scaffold protein is incorporated in the lipid bilayer and the biotin-binding protein is displayed outside the outer membrane vesicle.

Another aspect of the present disclosure relates to a therapeutic composition comprising: (i) an outer membrane vesicle comprising a lipid bilayer; (ii) a synthetic antigen receptor comprising an outer membrane scaffold protein fused to a biotin-binding protein, where the outer membrane scaffold protein is incorporated in the lipid bilayer and the biotin-binding protein is displayed outside the outer membrane vesicle; and (iii) a biotinylated antigen bound to the biotin-binding protein, where the therapeutic composition is administered to the mammal under conditions effective to elicit the immune response.

Another aspect of the present disclosure relates to a nucleic acid construct encoding a system for displaying antigens comprising: a first nucleic acid sequence encoding a synthetic antigen receptor comprising at least a portion of an outer membrane scaffold protein; and a second nucleic acid sequence encoding a biotin-binding protein, where said first nucleic acid sequence is coupled to said second nucleic acid sequence.

Another aspect of the present disclosure relates to an expression vector for generating a system for displaying antigens comprising a nucleic acid construct according to the present disclosure.

Another aspect of the present disclosure relates to a method of eliciting an immune response in a subject. This method involves administering a therapeutic composition comprising (i) an outer membrane vesicle comprising a lipid bilayer; (ii) a synthetic antigen receptor comprising at least a portion of an outer membrane scaffold protein fused to a biotin-binding protein, wherein the at least a portion of the outer membrane scaffold protein is incorporated in the lipid bilayer and the biotin-binding protein is displayed outside the outer membrane vesicle; and (iii) a biotinylated antigen bound to the biotin-binding protein, where the therapeutic composition is administered to the subject to elicit the immune response.

To develop a more universal strategy for tethering virtually any biomolecular cargo to the exterior of outer membrane vesicles (“OMVs”), a system for displaying antigens (i.e., the AddVax (avidin-based dock-and-display for vaccine antigen cross (x)-linking) platform), whereby biotinylated antigens are linked to the exterior of ready-made OMVs whose surfaces are remodeled with biotin-binding proteins, was created. This method involves producing OMV vectors that repetitively display multiple copies of a synthetic antigen receptor (SNARE) comprised of an outer membrane scaffold protein fused to a member of the avidin family. Following their production and isolation, SNARE-OMVs can be readily decorated with a wide range of biotinylated subunit antigens, including globular and membrane proteins, glycans and glycoconjugates, haptens, lipids, and short peptides. Importantly, antigen-studded SNARE-OMVs promote strong antigen-specific antibody responses that compare favorably to the responses measured for classically prepared OMV formulations (i.e., cellular expression of antigen-scaffold fusions). As demonstrated by the examples of the present disclosure, AddVax is a highly modular and versatile platform for on-demand vaccine creation that should enable rapid cycles of development, testing, and production of new OMV-based vaccines for numerous diseases.

### BRIEF DESCRIPTION OF THE DRAWINGS

FIGS. 1A-1B show a modular platform for rapid self-assembly of OMV-based vaccine candidates. FIG. 1A is a schematic illustration of AddVax technology whereby ready-made OMVs displaying a synthetic antigen receptor (SNARE-OMVs) are remodeled with biotinylated antigens-of-interest. Using AddVax, the surface of SNARE-OMVs can be remodeled with virtually any biomolecule that is amenable to biotinylation including peptides, proteins, carbohydrates, glycolipids, glycoproteins, haptens, lipids, and nucleic acids. FIG. 1B shows a schematic illustration of the genetic architecture of SNARE constructs evaluated in the Examples of the present disclosure. Numbers in parentheses denote amino acids of the scaffold that were fused to the biotin-binding eMA domain and used for membrane anchoring. Additional features include: export signal peptide from PelB (spPelB); c-Myc epitope tag (M); FLAG epitope tag (F), and NdeI, SphI, and NcoI restriction enzyme sites used for cloning.

FIGS. 2A-2B show the expression and antigen-binding activity of engineered SNAREs. FIG. 2A shows immunoblot analysis of OMV fractions isolated from hypervesiculating E. coli strain KPM404 ΔnlpI expressing each of the different SNAREs from plasmid pBAD24. An equivalent amount of SNARE-OMVs as determined by total protein assay was loaded in each lane. The blot was probed with anti-FLAG antibody (α-FLAG) to detect FLAG epitope (DYKDDDDK(SEQ ID NO:1)) located at the N- or C-terminus of each construct. Expected location of full-length SNARE fusion proteins are denoted by black arrows. Molecular weight (Mw) ladder is indicated at left. FIG. 2B is a graph showing binding of biotin-GFP to each of the different SNARE-OMVs as indicated. Binding activity was determined by ELISA in which biotin-binding SNARE-OMVs were immobilized on plates and subjected to varying amounts of biotin-GFP, after which plates were extensively washed prior to detection of bound biotin-GFP using anti-polyhistidine antibody to detect C-terminal 6xHis tag on GFP. Controls were performed by treating the same set of SNARE-OMVs with unmodfied GFP in place of biotin-GFP. All data were normalized to the maximum signal correpsonding to the eMA-IgAPβ construct in the presence of 10 nM biotin-GFP. Datapoints represent the average of three biological replicates and error bars represent the standard deviation of the mean.

FIGS. 3A-3C show the expression and purification of biotinylated protein antigens. FIGS. 3A and 3B show Coomassie blue-stained SDS-PAGE gels of: purified GFP and Sx-Cm-MOMP as well as their biotinylated counterparts (FIG. 3A); and purified Pfs25 (FIG. 3B). GFP and Sx-Cm-MOMP were both produced using E. coli BL21(DE3), which yielded ˜100 mg/L of GFP and ˜5 mg/L of Sx-Cm-MOMP. Note that GFP was purified by nickel only, whereas SIMPLEx-MOMP was purified by Ni-NTA resin followed by amylose. Pfs25 was produced using a baculovirus expression system involving SF9 cells and P2 virus, which yielded ˜25 mg/L of Pfs25 using Ni-NTA resin. FIG. 3C shows immunoblot analysis of CRM197 with four tandemly repeated DQNAT (SEQ ID NO:2) glycosylation motifs purified from E. coli CLM24 with (left lane) or without (right lane) plasmid DNA encoding the FtO-PS biosynthetic machinery. Glyconjugate yields were typically 2-3 mg/L. Blots were probed with anti-polyhistidine antibody (α6xHis) to detect the CRM197 carrier protein or FB11 (αFtO-PS) to detect the FtO-PS glycan. Image shows merge of α6x-His and αFtO-PS signals. High molecular weight laddering for red signal is characteristic of variable chain length O-PS polymers that are seen in native FtLPS as well as in glycoconjugates derived from engineered E. coli (Stark et al., “On-Demand Biomanufacturing of Protective Conjugate Vaccines,” Sci. Adv. 7(6):eabe9444 (2021), which is hereby incorporated by reference in its entirety). All images are representative of at least three biological replicates. Molecular weight (Mw) markers are shown at the left of each image.

FIGS. 4A-4E demonstrate the optimization of biotin-GFP docking on eMA-IgAPβ and Lpp-OmpA-eMA SNAREs. FIG. 4A shows graphs of the binding of biotin-GFP to SNARE-OMVs isolated from hypervesiculating E. coli strain KPM404 ΔnlpI expressing Lpp-OmpA-eMA or eMA-IgAPβ from plasmid pBAD24 and induced at low (Abs600˜0.6), medium (Abs600˜1.2), or high (Abs600˜1.8) culture density. Data in both graphs were normalized to the maximum binding signal corresponding to Lpp-OmpA-eMA SNARE-OMVs (high induction case) in the presence of 3.3 nM biotin-GFP. FIG. 4B show graphs of cell growth for same cultures in FIG. 4A where cell density was measured at time of induction (white bars) and just prior to harvesting SNARE-OMVs (gray bars). FIG. 4C show graphs of biotin-GFP binding and cell growth as in FIGS. 4A and 4B but with 50-fold lower L-arabinose (L-ara) inducer for cells expressing Lpp-OmpA-eMA. Binding data were normalized to the maximum binding signal corresponding to the Lpp-OmpA-eMA SNARE-OMVs in the presence of 3.3 nM biotin-GFP. FIG. 4D shows graphs of (left panel) biotin-GFP binding for SNARE-OMVs isolated from cells expressing Lpp-OmpA-eMA or eMA-IgAPβ from plasmid pTrham in the presence of different amounts of L-rhamnose (1-rha) as indicated, and (right panel) cell growth for a subset of the cells in left panel. FIG. 4E is a graphical comparison of biotin-GFP binding for Lpp-OmpA-eMA expressed from pBAD24 versus pTrham with inducer amounts as indicated. Data in FIG. 4D and FIG. 4E were normalized to the maximum binding signal corresponding to the Lpp-OmpA-eMA construct in the presence of 0.5 mM L-rha. Binding activity in all panels was determined by ELISA in which SNARE-OMVs were immobilized on plates and subjected to varying amounts of biotin-GFP, after which plates were extensively washed prior to detection of bound biotin-GFP using anti-polyhistidine antibody to detect C-terminal 6xHis tag on GFP. All binding data are the average of three biological replicates and error bars represent the standard deviation of the mean.

FIGS. 5A-5B show expression and antigen-binding activity of SNAREs with alternative biotin-binding modules. FIG. 5A shows graphs of binding of biotin-GFP to each of the different SNARE-OMVs and cell growth, as indicated. Binding activity was determined by ELISA in which biotin-binding SNARE-OMVs were immobilized on plates and subjected to varying amounts of biotin-GFP, after which plates were extensively washed prior to detection of bound biotin-GFP using anti-polyhistidine antibody to detect C-terminal 6xHis tag on GFP (left panel). Controls were performed by treating the same set of SNARE-OMVs with unmodfied GFP in place of biotin-GFP. All data were normalized to the maximum signal corresponding to the Lpp-OmpA-eMA construct in the presence of 1 nM biotin-GFP. Datapoints represent the average of three biological replicates and error bars represent the standard deviation of the mean. FIG. 5A also shows a graph (right panel) of cell growth for same cultures in FIG. 5A (left panel) where cell density was measured at time of induction (white bars) and just prior to harvesting SNARE-OMVs (gray bars). FIG. 5B shows immunoblot analysis of OMV fractions isolated from hypervesiculating E. coli strain KPM404 ΔnlpI expressing each of the different SNAREs from plasmid pBAD24. An equivalent amount of SNARE-OMVs as determined by total protein assay was loaded in each lane. Blot was probed with anti-FLAG antibody (α-FLAG) to detect FLAG epitope (DYKDDDDK (SEQ ID NO:1)) located at the C-terminus of each construct. Expected location of full-length SNARE fusion proteins are denoted by black arrows. Molecular weight (Mw) ladder is indicated at left.

FIGS. 6A-6D demonstrate that chimeric Lpp-OmpA-eMA SNARE enables controllable antigen loading on OMVs. FIG. 6A is a graph showing the dose-response curve generated by loading biotin-GFP or unmodified GFP on SNARE-OMVs isolated from hypervesiculating E. coli strain KPM404 ΔnlpI expressing the Lpp-OmpA-eMA construct from plasmid pTrham (induced with 0.5 mM L-rhamnose). Blank OMVs were isolated from plasmid-free KPM404 ΔnlpI cells. Binding activity was determined by ELISA in which Lpp-OmpA-eMA SNARE-OMVs were immobilized on plates and subjected to varying amounts of biotin-GFP, after which plates were extensively washed prior to detection of bound biotin-GFP using anti-polyhistidine antibody to detect C-terminal 6xHis tag on GFP. Data were normalized to the maximum binding signal corresponding to Lpp-OmpA-eMA SNARE-OMVs in the presence of 3.3 nM biotin-GFP. FIG. 6B is a graph of the same OMVs as in FIG. 6A, but dose-response was generated by first incubating OMVs with biotin-GFP or unmodified GFP in solution, washing to remove unbound protein, and determining GFP levels by ELISA-based detection. FIG. 6C is a graph showing a comparison of GFP levels on Lpp-OmpA-eMA SNARE-OMVs versus ClyA-GFP OMVs. ClyA-GFP OMVs were isolated from KPM404 ΔnlpI cells expressing ClyA-GFP fusion construct from plasmid pBAD18 as described in Kim et al., “Engineered Bacterial Outer Membrane Vesicles with Enhanced Functionality,” J. Mol. Biol. 380:51-66 (2008), which is hereby incorporated by reference in its entirety. Binding data are the average of triplicate measurements, and all error bars represent the standard deviation of the mean. FIG. 6D shows transmission electron micrographs of Lpp-OmpA-eMA SNARE-OMVs alone or following incubation with unmodified GFP or biotin-GFP as indicated. The scale bar represents 200 nm.

FIGS. 7A-7J demonstrate the rapid self-assembly of OMV vaccine candidates decorated with diverse biomolecular antigens. FIG. 7A-7J are graphs showing dose-response curves generated by loading biotinylated or non-biotinylated antigens on SNARE-OMVs isolated from hypervesiculating KPM404 ΔnlpI cells expressing the Lpp-OmpA-eMA construct from plasmid pTrham (induced with 0.5 mM L-rhamnose). Blank OMVs were isolated from plasmid-free KPM404 ΔnlpI cells. Binding activity was determined by ELISA in which Lpp-OmpA-eMA SNARE-OMVs were immobilized on plates and subjected to varying amounts of unbiotinylated or biotinylated antigen, after which plates were extensively washed prior to detection of bound antigen using the antibodies indicated at top of each panel. Anti-6x-His antibody was used to detect Pfs25 membrane-anchored protein (FIG. 7A), Sx-Cm-MOMP integral membrane protein (FIG. 7B), CRM197-FtO-PS glycoconjugate (FIG. 7C), and B6016-M30 peptide (FIG. 7F) antigens; anti-FtLPS was used to detect CRM197-FtO-PS (FIG. 7D) and FtO-PS glycan (FIG. 7E); anti-GD2 antibody was used to detect GD2 glycan (FIG. 7G); anti-Le^{Y} was used to detect Le^{Y} glycan (FIG. 7H); anti-DNP was used to detect DNP hapten (FIG. 7I); and anti-PC was used to detect PC lipid (FIG. 7J). Data were normalized to the maximum binding signal in each experiment. All binding data are the average of triplicate measurements and error bars represent the standard deviation of the mean.

FIGS. 8A-8B demonstrate that SNARE-OMVs decorated with biotin-GFP boost GFP-specific IgG titers. FIG. 8A is a graph of GFP-specific IgG titers in endpoint (day 56) serum of individual mice (gray dots) and geometric mean titers of each group (horizontal black lines). Five groups of BALB/c mice, seven mice per group, were immunized s.c. with the following: PBS, SNARE-OMVs isolated from KPM404 ΔnlpI cells expressing the Lpp-OmpA-eMA construct, SNARE-OMVs mixed with non-biotinylated or biotinylated GFP, and ClyA-GFP isolated from KPM404 ΔnlpI cells expressing ClyA-GFP fusion. Mice received prime injections containing an equivalent amount of OMVs (20 μg total protein) on day 0 and were boosted on day 21 and day 42 with the same doses. FIG. 8B is a graph showing the geometric mean IgG subclass titers measured from endpoint serum with IgG1 titers in dark gray and IgG2a in light gray. Statistical significance of antibody titers for SNARE-OMVs+biotin-GFP against blank SNARE-OMVs and SNARE-OMVs+GFP indicates statistically significant difference (p<0.0001 and p<0.01, respectively; unpaired t test with Welch's correction) between the groups; ns—not significant.

FIGS. 9A-9E demonstrate that SNARE-OMVs decorated with biotinylated Sx-Cm-MOMP elicit pathogen-specific IgGs. FIG. 9A is a schematic of SIMPLEx strategy for converting integral membrane proteins into water-soluble proteins that can be expressed at high titers in the cytoplasm of host cells. Here, the β-barrel outer membrane protein Cm-MOMP was fused at its N-terminus with E. coli maltose-binding protein (MBP) and at its C-terminus with truncated ApoAI (ApoAI*). Structural analysis indicates that ApoAI* adopts a belt-like conformation around the membrane helices of proteins to which it is fused, effectively shielding these highly hydrophobic segments from water (Mizrachi et al., “Making Water-Soluble Integral Membrane Proteins in vivo using an Amphipathic Protein Fusion Strategy,” Nat. Commun. 6:6826 (2015), which is hereby incorporated by reference in its entirety). FIG. 9B (left blot) shows the antigenicity of an Sx-Cm-MOMP construct evaluated by immunoblot analysis using mAb MoPn-40. Native Cm-MOMP (nCm-MOMP) served as a positive control while Sx-CtE-MOMP served as a negative control. FIG. 9B (right blot) shows that the latter construct was detected with commercial antibody specific for CtE-MOMP, which did not react with Sx-Cm-MOMP or nMOMP. Expected location of full-length SIMPLEx fusion proteins are denoted by black arrows. Molecular weight (M_{w}) ladder is indicated at left. FIG. 9C is a graph showing total IgG titers against recombinant preparations of Cm-MOMP (rCm-MOMP) in endpoint (day 56) serum of individual mice (gray dots) and median titers of each group (horizontal black lines). Three groups of BALB/c mice, seven mice per group, were immunized s.c. with the following: PBS, SNARE-OMVs isolated from KPM404 ΔnlpI cells expressing the Lpp-OmpA-eMA construct, and SNARE-OMVs mixed with biotinylated Sx-Cm-MOMP. Mice received prime injections containing an equivalent amount of OMVs (20 μg total protein) on day 0 and were boosted on day 21 and day 42 with the same doses. FIG. 9D and FIG. 9E are the same as in FIG. 9C, but with either a native preparation of Cm-MOMP (nCm-MOMP) (FIG. 9D) or elementary bodies (EBs) (FIG. 9E) as immobilized antigens. Statistical significance of antibody titers for SNARE-OMVs+biotin-Sx-Cm-MOMP against blank SNARE-OMVs and PBS indicates statistically significant differences (p<0.0001 for ELISAs with rCm-MOMP and nCm-MOMP; p<0.01 for ELISA with EBs; unpaired t test with Welch's correction) between the groups.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the disclosure.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of”.

Terms of degree such as “substantially,” “about,” and “approximately” and the symbol “—” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “—”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

The term “subject” is inclusive of the definition of the term “patient” and inclusive of the term “healthy subject” (i.e., an individual (e.g., a human) who is entirely normal in all respects or with respect to a particular condition.

The term “patient” means a subject (preferably a human) who has presented a clinical manifestation of a particular symptom or symptoms suggesting the need for treatment, who is treated preventatively or prophylactically for a condition, or who has been diagnosed with a condition to be treated.

The terms “treat”, “treatment of”, “treating” and the like refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For example, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

Suitable subjects in accordance with the methods described herein include, without limitation, a mammal, e.g., a human. In certain embodiments, the subject is an infant, a child, an adolescent, a young adult, an adult, or a geriatric adult. Additional suitable subjects include, but are not limited to, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

Engineered outer membrane vesicles (OMVs) derived from laboratory strains of bacteria are a promising technology for the creation of non-infectious, nanoparticle vaccines against diverse pathogens. As mimics of the bacterial cell surface, OMVs offer a molecularly-defined architecture for programming repetitive, high-density display of heterologous antigens in conformations that elicit strong B and T cell immune responses. However, antigen display on the surface of OMVs can be difficult to control and highly variable due to bottlenecks in protein expression and localization to the outer membrane of the host cell, especially for bulky and/or complex antigens. To address this shortcoming, the present application describes a universal approach called AddVax (avidin-based dock-and-display for vaccine antigen cross (x)-linking) whereby virtually any antigen that is amenable to biotinylation can be linked to the exterior of OMVs whose surfaces are remodeled with multiple copies of a synthetic antigen receptor (SNARE) comprised of an outer membrane scaffold protein fused to a member of the avidin family. As shown herein, SNARE-OMVs can be readily decorated with a molecularly diverse array of biotinylated subunit antigens, including globular and membrane proteins, glycans and glycoconjugates, haptens, lipids, and short peptides. When the resulting OMV formulations were injected in wild-type BALB/c mice, strong antigen-specific antibody responses were observed that depended on the physical coupling between the antigen and SNARE-OMV delivery vehicle. Overall, these results demonstrate AddVax as a modular platform for rapid self-assembly of antigen-studded OMVs with the potential to accelerate vaccine generation, respond rapidly to pathogen threats in humans and animals, and simplify vaccine stockpiling.

Accordingly, one aspect of the present disclosure relates to a system for displaying antigens. This system includes an outer membrane vesicle comprising a lipid bilayer and a synthetic antigen receptor comprising an outer membrane scaffold protein fused to a biotin-binding protein, where the outer membrane scaffold protein is incorporated in the lipid bilayer and the biotin-binding protein is displayed outside the outer membrane vesicle.

The terms “outer membrane vesicle” or “OMV” refers to a spherical nanostructure (˜20-250 nm) produced by gram-negative bacteria. OMVs are composed of proteins, lipids, and glycans, including LPS, derived primarily from the bacterial periplasm and outer membrane. As described herein, OMVs are nonreplicating, immunogenic mimics of their parental bacteria that stimulate both innate and adaptive immunity and possess intrinsic adjuvant properties (see, e.g., Chen et al., “Outer Membrane Vesicle Displaying Engineered Glycotopes Elicit Protective Antibodies,” PNASE 113(26): E3609-E3618, which is hereby incorporated by reference in its entirety).

The term “synthetic antigen receptor” or “SNARE” refers to a fusion protein comprising at least a portion of an outer membrane scaffold protein and a biotin binding protein.

The term “outer membrane scaffold protein” refers to an integral membrane protein (e.g., a virulence factor such as E. coli Cytolysin A) or a portion thereof which is sufficient to be associated with an outer membrane vesicle.

The outer membrane scaffold protein may have a structure sufficient to be imbedded into the lipid bilayer of the outer membrane vesicle. In some embodiments, the outer membrane scaffold protein comprises a beta domain or a portion which forms a beta strand and/or beta barrel, e.g., the β domain of an autotransporter protein, the β domain of a pore forming toxin, or Lpp-OmpA.

Autotransporter proteins are bacterial virulence factors that contain an N-terminal extracellular (“passenger”) domain and a C-terminal β barrel (“β”) domain that anchors the protein to the outer membrane. Exemplary suitable autotransporter proteins include, without limitation, adhesin involved in diffuse adherence (“AIDA-I”), antigen-43 (“Ag43”), haemoglobin binding protease (“HbP”), immunoglobulin A1 protease (“IgA1”), and intimin (“Int”). Additional suitable autotransporter proteins are identified in Clarke et al., “Phylogenic Classification and Functional Review of Autotransporters,” Front. Immunol. 13:921272 (2022), which is hereby incorporated by reference in its entirety.

Pore forming toxins are virulence factors that oligomerize upon binding to cellular membrane and convert to stable membrane-integrated pores. Exemplary suitable pore forming toxins include, without limitation, cytolysin A (“ClyA”).

Lpp-OmpA comprises a lipoprotein signal peptide and the first nine N-terminal amino acids of the E. coli lipoprotein (“Lpp”) attached to a transmembrane domain (amino acids 46-159) from outer membrane protein A (“OmpA”) (see, e.g., Francisco et al., “Production and Fluorescence-Activated Cell Sorting of Escherichia coli Expressing a Functional Antibody Fragment on the External Surface,” Proc. Natl. Acad. Sci. USA 90(22):10444-10448, which is hereby incorporated by reference in its entirety).

In some embodiments, the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), the C-terminal domain of adhesin involved in diffuse adherence (AIDA-I), and derivatives thereof.

As described herein supra, the outer membrane scaffold protein may comprise a portion which forms a beta strand and/or beta barrel. In accordance with such embodiments, the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), AIDA-I (962-1286, SEQ ID NO:15), and derivatives thereof.

The amino acid sequence for intimin (1-659) is SEQ ID NO:3, as follows:

MITHGCYTRTRHKHKLKKTLIMLSAGLGLFEYVNQNSFANGENYFKLGSDSKLLTHDSYQNRLE
YTLKTGETVADLSKSQDINLSTIWSLNKHLYSSESEMMKAAPGQQIILPLKKLPFEYSALPLLG
SAPLVAAGGVAGHTNKLTKMSPDVTKSNMTDDKALNYAAQQAASLGSQLQSRSLNGDYAKDTAL
GIAGNQASSQLQAWLQHYGTAEVNLQSGNNFDGSSLDFLLPFYDSEKMLAFGQVGARYIDSRFT
ANLGAGQRFFLPANMLGYNVFIDQDFSGDNTRLGIGGEYWRDYFKSSVNGYFRMSGWHESYNKK
DYDERPANGFDIRFNGYLPSYPALGAKLIYEQYYGDNVALFNSDKLQSNPGAATVGVNYTPIPL
VTMGIDYRHGTGNENDLLYSMQFRYQFDKSWSQQIEPQYVNELRTLSGSRYDLVQRNNNIILEY
KKQDILSLNIPHDINGTEHSTQKIQLIVKSKYGLDRIVWDDSALRSQGGQIQHSGSQSAQDYQA
ILPAYVQGGSNIYKVTARAYDRNGNSSNNVQLTITVLSNGQVVDQVGVTDFTADKTSAKADNAD
TITYTATVKKNGVAQANVPVSFNIVSGTATLGANSAKTDANGKATVTLKSSTPGQVVVSAKTAE
MTSALNASAVIFFDQTKAS

The nucleotide sequence for intimin (1-659) is SEQ ID NO:4, as follows:

ATGATCACCCACGGTTGCTACACCCGTACCCGTCACAAGCACAAACTGAAGAAAACCCTGATTA
TGCTGAGCGCGGGTCTGGGCCTGTTCTTTTACGTTAACCAGAACAGCTTCGCGAACGGCGAGAA
CTATTTTAAGCTGGGCAGCGACAGCAAACTGCTGACCCACGATAGCTACCAGAACCGTCTGTTC
TATACCCTGAAAACCGGTGAAACCGTGGCGGACCTGAGCAAGAGCCAAGATATCAACCTGAGCA
CCATTTGGAGCCTGAACAAACACCTGTACAGCAGCGAGAGCGAAATGATGAAGGCGGCGCCGGG
CCAGCAAATCATTCTGCCGCTGAAGAAACTGCCGTTTGAGTATAGCGCGCTGCCGCTGCTGGGT
AGCGCGCCGCTGGTTGCGGCGGGTGGCGTGGCGGGCCACACCAACAAGCTGACCAAAATGAGCC
CGGACGTTACCAAAAGCAACATGACCGACGATAAAGCGCTGAACTATGCGGCGCAGCAAGCGGC
GAGCCTGGGTAGCCAGCTGCAAAGCCGTAGCCTGAACGGCGACTATGCGAAAGATACCGCGCTG
GGTATCGCGGGCAACCAAGCGAGCAGCCAGCTGCAAGCGTGGCTGCAGCACTACGGCACCGCGG
AAGTGAACCTGCAAAGCGGTAACAACTTCGACGGCAGCAGCCTGGATTTCCTGCTGCCGTTTTA
CGACAGCGAAAAAATGCTGGCGTTTGGTCAAGTGGGTGCGCGTTATATTGATAGCCGTTTTACC
GCGAACCTGGGTGCGGGCCAGCGTTTCTTTCTGCCGGCGAACATGCTGGGTTACAACGTTTTCA
TCGACCAAGATTTTAGCGGTGACAACACCCGTCTGGGCATTGGTGGCGAATACTGGCGTGATTA
TTTCAAAAGCAGCGTGAACGGTTATTTTCGTATGAGCGGCTGGCACGAGAGCTACAACAAGAAA
GACTATGATGAACGTCCGGCGAACGGTTTCGACATCCGTTTTAACGGCTACCTGCCGAGCTATC
CGGCGCTGGGTGCGAAACTGATTTACGAGCAGTACTATGGCGACAACGTTGCGCTGTTCAACAG
CGATAAGCTGCAAAGCAACCCGGGTGCGGCGACCGTTGGCGTGAACTACACCCCGATCCCGCTG
GTGACGATGGGTATTGACTATCGTCACGGCACCGGCAACGAAAACGATCTGCTGTACTCCATGC
AGTTCCGTTATCAGTTTGACAAGAGCTGGAGCCAGCAAATCGAGCCGCAGTACGTTAACGAACT
GCGTACCCTGAGCGGCAGCCGTTATGATCTGGTGCAGCGTAACAACAACATCATTCTGGAGTAC
AAGAAACAAGACATTCTGAGCCTGAACATCCCGCACGATATTAACGGCACCGAACACAGCACCC
AGAAGATCCAACTGATCGTTAAGAGCAAGTACGGCCTGGACCGTATCGTGTGGGACGATAGCGC
GCTGCGTAGCCAGGGTGGCCAGATTCAACACAGCGGTAGCCAGAGCGCGCAAGATTACCAGGCG
ATCCTGCCGGCGTATGTTCAAGGTGGCAGCAACATTTACAAGGTGACCGCGCGTGCGTATGACC
GTAACGGCAACAGCAGCAACAACGTTCAGCTGACCATCACCGTGCTGAGCAACGGTCAAGTGGT
TGATCAGGTTGGCGTGACCGACTTCACCGCGGATAAGACCAGCGCGAAAGCGGACAACGCGGAT
ACCATCACCTACACCGCGACCGTTAAGAAAAACGGTGTGGCGCAGGCGAACGTTCCGGTGAGCT
TTAACATTGTGAGCGGCACCGCGACCCTGGGTGCGAACAGCGCGAAGACCGACGCGAACGGTAA
AGCGACCGTGACCCTGAAGAGCAGCACCCCGGGTCAAGTGGTTGTGAGCGCGAAAACCGCGGAG
ATGACCAGCGCGCTGAACGCGAGCGCGGTGATCTTCTTTGATCAGACCAAGGCGAGC

The amino acid sequence for cytolysin (ClyA) is SEQ ID NO:5, as follows:

MTEIVADKTVEVVKNAIETADGALDLYNKYLDQVIPWQTFDETIKELSRFKQEYSQAASVLVGD
IKTLLMDSQDKYFEATQTVYEWCGVATQLLAAYILLFDEYNEKKASAQKDILIKVLDDGITKLN
EAQKSLLVSSQSFNNASGKLLALDSQLTNDFSEKSSYFQSQVDKIRREAYAGAAAGVVAGPFGL
IISYSIAAAVVEGKLIPELKNKLKSVQNFFTTLSNTVKQANKDIDAAKLKLTTEIAAIGEIKTE
TETTRFYVDYDDLMLSLLKEAAKKMINTCNEYQKRHGKKTLFEVPEV

The nucleotide sequence for cytolysin (ClyA) is SEQ ID NO:6, as follows:

ATGACCGAGATTGTGGCGGACAAAACCGTTGAGGTGGTTAAGAACGCGATCGAAACCGCGGACG
GTGCGCTGGATCTGTACAACAAATATCTGGACCAAGTGATTCCGTGGCAAACCTTCGATGAAAC
CATCAAAGAACTGAGCCGTTTTAAGCAGGAATACAGCCAAGCGGCGAGCGTGCTGGTTGGTGAT
ATTAAAACCCTGCTGATGGACAGCCAGGATAAGTACTTCGAGGCGACCCAAACCGTGTATGAAT
GGTGCGGTGTTGCGACCCAGCTGCTGGCGGCGTACATTCTGCTGTTTGACGAGTATAACGAAAA
GAAAGCGAGCGCGCAAAAAGATATCCTGATTAAGGTGCTGGACGATGGTATCACCAAACTGAAC
GAGGCGCAGAAGAGCCTGCTGGTTAGCAGCCAAAGCTTCAACAACGCGAGCGGCAAGCTGCTGG
CGCTGGACAGCCAGCTGACCAACGATTTCAGCGAGAAAAGCAGCTACTTTCAGAGCCAAGTGGA
CAAGATCCGTCGTGAAGCGTATGCGGGTGCGGCGGCGGGCGTGGTTGCGGGTCCGTTTGGCCTG
ATCATTAGCTACAGCATTGCGGCGGCGGTGGTTGAGGGCAAACTGATCCCGGAACTGAAGAACA
AACTGAAGAGCGTGCAGAACTTCTTTACCACCCTGAGCAACACCGTTAAACAAGCGAACAAGGA
CATTGATGCGGCGAAACTGAAGCTGACCACCGAGATCGCGGCGATTGGTGAAATCAAGACCGAA
ACCGAAACCACCCGTTTCTACGTTGATTATGACGATCTGATGCTGAGCCTGCTGAAAGAGGCGG
CGAAGAAAATGATCAACACCTGCAACGAATATCAGAAGCGTCACGGCAAGAAAACCCTGTTTGA
GGTGCCGGAAGTT

The amino acid sequence for Lpp-OmpA is SEQ ID NO:7, as follows:

MKATKLVLGAVILGSTLLAGCSSNAKIDQGINPYVGFEMGYDWLGRMPYKGSVENGAYKAQGVQ
LTAKLGYPITDDLDIYTRLGGMVWRADTKSNVYGKNHDTGVSPVFAGGVEYAITPEIATRLEYQ
WTNNIGDAHTIGTRPDN

The nucleotide sequence for Lpp-OmpA is SEQ ID NO:8, as follows:

ATGAAGGCGACCAAACTGGTGCTGGGTGCGGTTATTCTGGGCAGCACCCTGCTGGCGGGTTGCA
GCAGCAACGCGAAAATCGACCAGGGCATTAACCCGTACGTGGGTTTCGAAATGGGCTATGATTG
GCTGGGTCGTATGCCGTACAAGGGTAGCGTGGAGAACGGCGCGTATAAAGCGCAGGGTGTTCAA
CTGACCGCGAAGCTGGGCTACCCGATCACCGACGATCTGGACATTTATACCCGTCTGGGTGGCA
TGGTGTGGCGTGCGGACACCAAGAGCAACGTTTACGGTAAAAACCACGATACCGGCGTGAGCCC
GGTTTTTGCGGGTGGCGTTGAGTACGCGATCACCCCGGAAATTGCGACCCGTCTGGAGTATCAA
TGGACCAACAACATCGGTGACGCGCACACCATTGGCACCCGTCCGGATAAC

The amino acid sequence for HbpΔβ (1091-1377) is SEQ ID NO:9, as follows:

SYNNFITEVGSLNKRMGDLRDINGEAGTWVRLLNGSGSADGGETDHYTLLQMGADRKHELGSMD
LFTGVMATYTDTDASADLYSGKTKSWGGGFYASGLFRSGAYFDVIAKYIHNENKYDLNFAGAGK
QNFRSHSLYAGAEVGYRYHLTDTTFVEPQAELVWGRLQGQTFNWNDSGMDVSMRRNSVNPLVGR
TGVVSGKTFSGKDWSLTARAGLHYEFDLTDSADVHLKDAAGEHQINGRKDSRMLYGVGLNARFG
DNTRLGLEVERSAFGKYNTDDAINANIRYSF

The nucleotide sequence for HbpΔβ (1091-1377) is SEQ ID NO:10, as follows:

AGCTATAACAACTTTATCACCGAGGTGGGCAGCCTGAACAAGCGTATGGGTGACCTGCGTGATA
TTAACGGCGAAGCGGGCACCTGGGTTCGTCTGCTGAACGGCAGCGGTAGCGCGGATGGTGGCTT
TACCGACCACTACACCCTGCTGCAGATGGGCGCGGATCGTAAACACGAGCTGGGCAGCATGGAC
CTGTTCACCGGTGTGATGGCGACCTATACCGACACCGATGCGAGCGCGGATCTGTACAGCGGCA
AGACCAAAAGCTGGGGTGGCGGTTTCTATGCGAGCGGCCTGTTTCGTAGCGGTGCGTACTTCGA
TGTGATCGCGAAGTATATTCACAACGAGAACAAATACGACCTGAACTTTGCGGGCGCGGGCAAG
CAGAACTTTCGTAGCCACAGCCTGTATGCGGGTGCGGAAGTTGGTTACCGTTATCACCTGACCG
ACACCACCTTTGTGGAGCCGCAAGCGGAACTGGTTTGGGGCCGTCTGCAGGGTCAAACCTTCAA
CTGGAACGATAGCGGCATGGACGTGTCCATGCGTCGTAACAGCGTGAACCCGCTGGTTGGCCGT
ACCGGTGTGGTTAGCGGCAAGACCTTTAGCGGTAAAGATTGGAGCCTGACCGCGCGTGCGGGTC
TGCACTATGAGTTCGACCTGACCGATAGCGCGGACGTTCACCTGAAGGATGCGGCGGGCGAACA
CCAAATCAACGGTCGTAAAGACAGCCGTATGCTGTACGGCGTGGGTCTGAACGCGCGTTTTGGC
GACAACACCCGTCTGGGTCTGGAAGTGGAACGTAGCGCGTTCGGTAAATATAACACCGACGATG
CGATCAACGCGAACATTCGTTACAGCTTC

The amino acid sequence for Ag43 (700-1039) is SEQ ID NO:11, as follows:

LRSENAYRAEVPLYASMLTQAMDYDRILAGSRSHQTGVNGENNSVRLSIQGGHLGHDNNGGIVR
GATPESSGSYGFVRLEGDLLRTEVAGMSLTTGVYGAAGHSSVDVKDDDGSRAGTVRDDAGSLGG
YLNLVHTSSGLWADIVAQGTRHSMKASSDNNDFRARGRGWQGSLETGLPFSITDNLMLEPQLQY
TWQGLSLDDGQDNAGYVKFGHGSAQHVRAGFRLGSHNDMTFGEGTSSRDTLRDSTKHGVSELPV
NWWVQPSVIRTFSSRGDMSMGTAAAGSNMTFSPSRNGTSLDLQAGLEARVRENITLGVQAGYAH
SVSGNSAEGYNGQATLNVTF

The nucleotide sequence for Ag43 (700-1039) is SEQ ID NO:12, as follows:

CTGCGTAGCGAGAACGCGTACCGTGCGGAAGTGCCGCTGTATGCGTCCATGCTGACCCAGGCGA
TGGATTACGACCGTATCCTGGCGGGTAGCCGTAGCCACCAAACCGGTGTTAACGGCGAGAACAA
CAGCGTGCGTCTGAGCATCCAGGGTGGCCACCTGGGTCACGATAACAACGGTGGCATTGTTCGT
GGCGCGACCCCGGAAAGCAGCGGTAGCTACGGCTTCGTTCGTCTGGAGGGTGACCTGCTGCGTA
CCGAAGTGGCGGGCATGAGCCTGACCACCGGTGTTTATGGCGCGGCGGGTCACAGCAGCGTGGA
TGTTAAAGATGATGATGGCAGCCGTGCGGGCACCGTTCGTGATGATGCGGGTAGCCTGGGTGGC
TATCTGAACCTGGTGCACACCAGCAGCGGTCTGTGGGCGGACATCGTTGCGCAAGGCACCCGTC
ACAGCATGAAAGCGAGCAGCGATAACAACGATTTTCGTGCGCGTGGTCGTGGCTGGCAGGGTAG
CCTGGAAACCGGTCTGCCGTTTAGCATTACCGATAACCTGATGCTGGAACCGCAGCTGCAATAC
ACCTGGCAGGGTCTGAGCCTGGATGACGGCCAAGACAACGCGGGTTATGTGAAGTTCGGTCACG
GCAGCGCGCAACATGTTCGTGCGGGTTTCCGTCTGGGTAGCCACAACGATATGACCTTTGGCGA
GGGCACCAGCAGCCGTGATACCCTGCGTGACAGCACCAAACACGGCGTGAGCGAACTGCCGGTG
AACTGGTGGGTTCAGCCGAGCGTGATCCGTACCTTCAGCAGCCGTGGCGATATGAGCATGGGCA
CCGCGGCGGCGGGCAGCAACATGACCTTTAGCCCGAGCCGTAACGGCACCAGCCTGGACCTGCA
AGCGGGCCTGGAGGCGCGTGTTCGTGAAAACATTACCCTGGGCGTGCAGGCGGGTTATGCGCAC
AGCGTTAGCGGTAACAGCGCGGAGGGCTATAACGGTCAAGCGACCCTGAACGTTACCTTT

The amino acid sequence for IgAP (1245-1532) is SEQ ID NO:13, as follows:

STNTNSALSDAMASTQSILLDTGASLTRHIAQKSRADAEKNSVWMSNTGYGRDYASAQYRRFSS
KRTQTQIGIDRSLSENMQIGGVLTYSDSQHTFDQAGGKNTFVQANLYGKYYLNDAWYVAGDIGA
GSLRSRLQTQQKANFNRTSIQTGLTLGNTLKINQFEIVPSAGIRYSRLSSADYKLGDDSVKVSS
MAVKTLTAGLDFAYRFKVGNLTVKPLLSAAYFANYGKGGVNVGGKSFAYKADNQQQYSAGAALL
YRNVTLNVNGSITKGKQLEKQKSGOIKIQIRF

The nucleotide sequence for IgAP (1245-1532) is SEQ ID NO:14, as follows:

AGCACCAACACCAACAGCGCGCTGAGCGATGCGATGGCGAGCACCCAGAGCATTCTGCTGGATA
CCGGTGCGAGCCTGACCCGTCACATTGCGCAAAAGAGCCGTGCGGACGCGGAGAAAAACAGCGT
GTGGATGAGCAACACCGGTTACGGCCGTGATTATGCGAGCGCGCAGTATCGTCGTTTCAGCAGC
AAACGTACCCAAACCCAGATCGGCATTGACCGTAGCCTGAGCGAAAACATGCAAATTGGTGGCG
TTCTGACCTACAGCGACAGCCAACACACCTTCGATCAGGCGGGTGGCAAAAACACCTTTGTGCA
GGCGAACCTGTATGGCAAGTACTATCTGAACGACGCGTGGTACGTGGCGGGCGATATTGGTGCG
GGCAGCCTGCGTAGCCGTCTGCAAACCCAGCAAAAAGCGAACTTCAACCGTACCAGCATCCAGA
CCGGTCTGACCCTGGGCAACACCCTGAAGATTAACCAATTTGAGATCGTGCCGAGCGCGGGTAT
CCGTTACAGCCGTCTGAGCAGCGCGGACTATAAGCTGGGCGACGATAGCGTGAAAGTTAGCAGC
ATGGCGGTTAAGACCCTGACCGCGGGTCTGGATTTCGCGTACCGTTTTAAAGTGGGCAACCTGA
CCGTTAAGCCGCTGCTGAGCGCGGCGTACTTCGCGAACTATGGTAAAGGTGGCGTGAACGTTGG
TGGCAAGAGCTTTGCGTACAAAGCGGATAACCAGCAACAATACAGCGCGGGTGCGGCGCTGCTG
TACCGTAACGTGACCCTGAACGTTAACGGTAGCATTACCAAGGGCAAACAACTGGAAAAGCAGA
AAAGCGGTCAAATCAAGATTCAGATCCGTTTT

The amino acid sequence for AIDA-I (962-1286) is SEQ ID NO:15, as follows:

QYRPENGSYATNMALANSLELMDLNERKQFRAMSDNTQPESASVWMKITGGISSGKLNDGQNKT
TTNQFINQLGGDIYKFHAEQLGDFTLGIMGGYANAKGKTINYTSNKAARNTLDGYSVGVYGTWY
QNGENATGLFAETWMQYNWFNASVKGDGLEEEKYNLNGLTASAGGGYNLNVHTWTSPEGITGEF
WLQPHLQAVWMGVTPDTHQEDNGTVVQGAGKNNIQTKAGIRASWKVKSTLDKDTGRRFRPYIEA
NWIHNTHEFGVKMSDDSQLLSGSRNQGEIKTGIEGVITQNLSVNGGVAYQAGGHGSNAISGALG
IKYSF

The nucleotide sequence for AIDA-I (962-1286) is SEQ ID NO:16, as follows

CAGTACCGTCCGGAAAACGGTTCTTATGCAACTAACATGGCGCTGGCGAACAGCCTGTTCCTGA
TGGATCTGAACGAACGTAAACAATTCCGCGCTATGAGCGACAATACTCAGCCAGAATCTGCGAG
CGTTTGGATGAAAATTACCGGTGGTATCTCTAGCGGCAAGCTGAACGATGGTCAGAACAAAACT
ACCACCAACCAATTCATTAACCAGCTGGGCGGTGACATTTACAAGTTCCACGCTGAACAGCTGG
GTGACTTTACGCTGGGCATCATGGGTGGTTACGCCAACGCGAAAGGCAAAACTATCAACTACAC
TAGCAACAAAGCAGCGCGCAATACGCTGGACGGTTACTCTGTGGGCGTTTACGGCACTTGGTAT
CAGAATGGTGAAAACGCCACGGGCCTGTTCGCGGAAACCTGGATGCAGTACAACTGGTTCAACG
CGTCTGTGAAAGGCGACGGTCTGGAAGAGGAAAAGTACAACCTGAACGGTCTGACTGCAAGCGC
TGGCGGCGGTTACAATCTGAACGTCCATACTTGGACCAGCCCGGAAGGTATCACCGGCGAATTT
TGGCTGCAACCGCACCTGCAGGCTGTCTGGATGGGCGTTACCCCGGACACCCACCAAGAAGATA
ATGGCACCGTTGTGCAGGGCGCAGGCAAAAACAATATCCAGACTAAAGCCGGTATCCGCGCGTC
CTGGAAAGTGAAATCTACGCTGGATAAAGACACCGGCCGTCGCTTCCGTCCGTACATCGAAGCG
AATTGGATTCACAACACTCACGAGTTCGGCGTGAAGATGTCTGATGACTCTCAGCTGCTGTCCG
GCAGCCGTAACCAAGGCGAAATCAAAACCGGCATCGAGGGTGTAATCACCCAGAACCTGTCTGT
TAACGGTGGCGTTGCGTATCAGGCAGGTGGTCATGGCTCCAACGCGATCAGCGGCGCTCTGGGC
ATCAAATACTCTTTT

The term “biotin-binding protein” may refer to any protein or peptide fragment thereof capable of binding to biotin. Suitable biotin binding proteins include, e.g., members of the avidin family and derivatives thereof. In some embodiments, the biotin binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}, and derivatives thereof.

In some embodiments, the biotin-binding protein is a monomeric biotin-binding protein. In accordance with such embodiments, the biotin-binding protein is selected from the group consisting of enhanced monoavidin (eMA) and monomeric streptavidin mSA^{S25H}.

The synthetic antigen receptor may be selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}. In some embodiments, the synthetic antigen receptor is selected from the group consisting of Lpp-OmpA-eMA, Lpp-OmpA-RA, and Lpp-OmpA-mSA^{S25H}.

Fusions between the outer membrane scaffold protein (or at least a portion of the outer membrane scaffold protein which is sufficient to be associated with an outer membrane vesicle) and the biotin-binding protein according to the present disclosure may be such that the amino acid sequence of the outer membrane scaffold protein (or at least a portion of the outer membrane scaffold protein which is sufficient to be associated with an outer membrane vesicle) is directly contiguous with the amino acid sequence of the biotin-binding protein. Alternatively, the at least a portion of the outer membrane scaffold protein portion may be coupled to the biotin-binding protein by way of a linker sequence such as the flexible 5-residue Gly linker described herein or the flexible linkers from an immunoglobulin disclosed in U.S. Pat. No. 5,516,637 to Huang et al., which is hereby incorporated by reference in its entirety. Thus, in some embodiments, the system according to the present disclosure further includes a peptide linker connecting the outer membrane scaffold protein (or at least a portion of the outer membrane scaffold protein which is sufficient to be associated with an outer membrane vesicle) and the biotin-binding protein.

Suitable linker sequences include, without limitation, GGGGS (SEQ ID NO:17), GGGGSGGGGS (SEQ ID NO:18), GGGGSGGGGSGGGGS (SEQ ID NO:19), GGGGSGGGGSGGGGSGGGGS (SEQ ID NO:20); GGGGGG (SEQ ID NO:21), GGGGGGGG (SEQ ID NO:22), and GSAGSAAGSGEF (SEQ ID NO:23).

Another aspect of the present disclosure relates to a therapeutic composition comprising: (i) an outer membrane vesicle comprising a lipid bilayer; (ii) a synthetic antigen receptor comprising an outer membrane scaffold protein fused to a biotin-binding protein, where the outer membrane scaffold protein is incorporated in the lipid bilayer and the biotin-binding protein is displayed outside the outer membrane vesicle; and (iii) a biotinylated antigen bound to the biotin-binding protein, where the therapeutic composition is administered to the mammal under conditions effective to elicit the immune response.

Suitable outer membrane scaffold proteins (and portions of the outer membrane scaffold protein which is sufficient to be associated with an outer membrane vesicle) for use in the compositions and methods of the present disclosure are described in more detail supra. In some embodiments of the therapeutic composition described herein, the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), domain of immunoglobulin A protease (IgAP), and the C-terminal domain of adhesin involved in diffuse adherence (AIDA-I).

In some embodiments, the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

Suitable biotin-binding proteins for use in the compositions and methods of the present disclosure are described in more detail supra. Accordingly, in some embodiments of the therapeutic composition described herein, the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}.

Suitable synthetic antigen receptors for use in the compositions and methods of the present disclosure are described in more detail supra. Thus, in some embodiments of the therapeutic composition described herein, the synthetic antigen receptor is selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}. In accordance with such embodiments, the synthetic antigen receptor may be selected from the group consisting of Lpp-OmpA-eMA, Lpp-OmpA-RA, and Lpp-OmpA-mSA^{S25H}.

In some embodiments, the therapeutic composition further comprises a peptide linker connecting the outer membrane scaffold protein and the biotin binding protein. Suitable peptide linkers for use in the compositions and methods of the present disclosure are described in more detail supra.

The term “antigen” refers to any substance that can induce an immune response against that substance. Suitable exemplary antigens include, without limitation, globular proteins, membrane proteins, glycans, glycoconjugates, haptens, saccharides, lipids, peptides, nucleic acids, and combinations thereof. Specific examples include proteins, peptides, and polysaccharides having functional groups such as amino groups, carboxyl groups, thiol groups, and hydroxyl groups at the ends or side chains. Further, the substance may be a naturally occurring substance, a synthesized substance, or a gene-expressed substance, and may be a complete molecule or a fragment. In the present disclosure, the type of antigen is appropriately selected according to a desired outcome, e.g., the induction of an immune response against a specific target.

A “biotinylated antigen” refers to an antigen which has been modified with biotin. Methods of biotinylating antigens are well known in the art and can be used without any limitation. In some embodiments, an antigen may be biotinylated by contacting and reacting a biotinylated reagent and an antigen solution to covalently bind biotin to the antigen, and then removing the unreacted biotinylated reagent by gel filtration, dialysis, or the like.

In some embodiments, the biotinylated antigen comprises a globular protein, a post-translationally modified protein, a membrane protein, a glycan, a glycoconjugate, a hapten, a saccharide, a lipid, a peptide, a nucleic acid, or combinations thereof.

The therapeutic composition may be directed against an infectious disease, a neoplastic condition, a cancer, or a self-antigen. In accordance with such embodiments, the antigen may comprise an antigenic portion of a globular protein, a membrane protein, a glycan, a glycoconjugate, a hapten, a saccharide, a lipid, a peptide, and/or a nucleic acid.

The antigen may be selected from the group consisting of a human antigen, a cancer antigen, a viral antigen, a bacterial antigen, a parasitic antigen, a fungal antigen, and an allergen.

In some embodiments, the antigen is a human antigen. Suitable exemplary human antigens include self-antigens and antigens associated with a cancer or tumor cell (e.g., tumor-associated antigens).

In some embodiments, the antigen is a self-antigen. As described herein, a “self-antigen” is an antigen against which the host elicits an unwanted immune response that contributes to tissue destruction and the damage of normal tissues. Suitable exemplary self-antigens include, without limitation, antigens associated with an autoimmune disease such as multiple sclerosis, rheumatoid arthritis, type 1 diabetes, psoriasis or other autoimmune disorders. Additional exemplary antigens may be associated with Crohn's disease and other inflammatory bowel diseases such as ulcerative colitis, systemic lupus erythematosus (SLE), autoimmune encephalomyelitis, myasthenia gravis (MG), Hashimoto's thyroiditis, Goodpasture's syndrome, pemphigus, Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune associated infertility, glomerulonephritis (for example crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin dependent diabetes mellitus), autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, and Guillain-Bare syndrome. In some embodiments, the antigen is a self-antigen associated with arteriosclerosis or Alzheimer's disease. In some embodiments, the self-antigen is associated with multiple sclerosis. In accordance with such embodiments, the antigen is phosphocholine (PC).

In some embodiments, the antigen is associated with a cancer or tumor cell.

In some embodiments, the antigen is a cancer associated antigen. Exemplary cancer associated peptide antigens include, without limitation, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, GAGE-9, BAGE-1, RAGE-1, LB33/MUM-1, PRAME, NAG, MAGE-B2, MAGE-B3, MAGE-B4, tyrosinase, brain glycogen phosphorylase, Melan-A, MAGE-C1, MAGE-C2, MAGE-C3, MAGE-C4, MAGE-05, NY-ESO-1, LAGE-1, SSX-1, SSX-2 (HOM-MEL-40), SSX-4, SSX-5, SCP-1 and CT-7. See, for example, PCT application publication no. W096/10577. Other examples will be known to one of ordinary skill in the art and can be used in the invention in a like manner as those disclosed herein.

In some embodiments, the antigen is a tumor associated carbohydrate antigen (TACA). Exemplary tumor associated carbohydrate antigens include, without limitation, Tn, sialyl Tn (STn), T antigen, Globo-H, Lewis Y (Le^{Y}), sialyl Lewis A (SLea), sialyl Lewis X (SLeX), GM2, GM3, fucosyl GM1, GD2, GD3.

Additional suitable cancer and tumor-associated antigens are identified in Table 1 below.

TABLE 1
Exemplary Cancer and Tumor-Associated Antigens

| Antigen | Disease |
|---|---|
| Epidermal growth factor | NSCLC, epithelial carcinoma, glioma |
| receptor (EGFR) | |
| Variant III of the epidermal | Glioblastoma |
| growth factor receptor | |
| (EGFRvIII) | |
| Human epidermal growth | Ovarian cancer, breast cancer, |
| factor 2 (HER2) | glioblastoma, colon cancer, |
| | osteosarcoma, medulloblastoma |
| Mesothelin (MSLN) | Mesothelioma, ovarian cancer, |
| | pancreatic adenocarcinoma |
| Prostate-specific membrane | Prostate cancer |
| antigen (PSMA) | |
| Carcinoembryonic | Pancreatic adenocarcinoma, breast |
| antigen (CEA) | cancer, colorectal carcinoma |
| Disialoganglioside 2 (GD2) | Neuroblastoma, melanoma, |
| | osteosarcoma, and small-cell |
| | lung cancer |
| GD3 | Melanoma |
| Mucin 1 (MUC1) | Seminal vesicle cancer |
| Lewis Y (Le^{Y}) | Small Cell Lung Cancer |
| B16-M30 peptide | B16F10 melanoma |
| Wilms' tumor 1 (WT1) | Breast cancer, endometrial cancer, |
| peptide | ovarian cancer, hepatocellular carcinoma, |
| | colorectal cancer, glioblastoma, glioma, |
| | melanoma, non-small cell lung cancer |
| Melanoma associated antigen | Melanoma, adenocarcinoma |
| (MAGE) | |
| carcinoembryonic | Colorectal carcinoma |
| antigen (CEA) | |
| Ganglioside GM3 | Lung cancer, brain cancer, melanoma |
| Mucin 1 (MUC1) | Epithelial adenocarcinomas such as lung, |
| | liver, colon, breast, pancreatic, and |
| | ovarian cancer |

See, e.g., Qi et al., “Wilms' Tumor 1 (WT1) Expression and Prognosis in Solid Cancer Patients: A Systemic Review and Meta-Analysis,” Sci. Rep. 5:8924 (2015); Wagner et al., “Colorectal Cancer Vaccines: Tumor-Associated Antigens vs Neoantigens,” World J. Gastroenterol. 24(48):5418-5432 (2018); Chen et al., “MUC1: Structure, Function, and Clinic Application in Epithelial Cancers,” Int. J. Mol. Sci. 22(12):6567 (2021); and Zheng et al., “Ganglioside GM3 and its Role in Cancer,” Curr. Med. Chem. 26(16):2933-2947 (2019), which are hereby incorporated by reference in their entirety.

In some embodiments, the antigen is an allergen. Suitable exemplary allergens may be derived from milk, eggs, fish, crustacean shellfish, tree nuts, peanuts, wheat, coconut, and soybeans. Examples of specific food allergens include, without limitation, the following: milk (Bosd4, Bosd5, and Bosd6), eggs (ovomucoid, ovalbumin, ovotransferrin, lysozyme, and alpha-livetin), fish (Gadm1, Gadm2, Gadm3, Sals1, Sals2, Sals3, Gadc1, and Xipg1), crustacean shellfish (Homa1, Homa3, Homa6, Penm1, Penm2, Penm3, Penm4, Penm6, Litv1, Litv2, Litv3, Litv4, and Chafl), tree nuts (Prudu3, Prudu4, Prudu5, Prudu6, Jugn1, Jugn2, Jugr1, Jugr2, Bere2, Bere1, Cass5, Cora 1.0401, Cora 1.0402, Cora 1.0403, Cora 1.0404, Coral 1, Cora8, Cora9, Anah1, pecan protein albumin 2S, and Litc1), peanuts (Arah1, Arah2, Arah3, Arah4, and Arah5), wheat (Tria12, Tria14, Tria18, and Trial9), coconut (CNP1), and soybeans (Glym1, Glym2, Glym3, Glym4, and Glym5). In some embodiments, the allergen is derived from peanuts. In accordance with such embodiments, the allergen comprises Arah2 or a fragment thereof.

In some embodiments, the antigen is a viral antigen. For example, the antigen may be derived from a virus selected from the group consisting of the family Retroviridae (for example human deficiency viruses, such as HIV-1 (also referred to as HTLV-III), HIV-II, LAC or IDLV-III/LAV or HIV-III and other isolates such as HIV-LP), Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses (for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses. Suitable viral antigens are identified in Table 2 below (see, e.g., US Patent Application Publication No. 2020/0397886 A1, which is hereby incorporated by reference in its entirety).

TABLE 2
Exemplary Viral Antigens

| Antigen | Virus |
|---|---|
| gp160, gp140, gp21, MPER | Human Immunodeficiency |
| | Virus-1 (HIV-1) |
| F protein (prefusion) | Respiratory Syncytial Virus (RSV) |
| HA | Influenza A and B |
| glycoprotein 350/220 (L4p350) | Epstein-Barr Virus (EBV) |
| gB; UL128, UL130, UL131A, | Cytomegalovirus (CMV) |
| gH (UL75), and gL (UL115) | |
| E protein | Dengue virus (DENV) |
| Spike (S) glycoprotein | Severe Acute Respiratory |
| | Syndrome (SARS) |
| Spoke (S) glycoprotein | Middle East respiratory |
| | syndrome (MERS) |
| EBOV | Ebola virus |
| Marbera GP or sGP | Marberg |
| Gn and Gc envelope | Hantaan virus |
| glycoproteins | |
| HepB surface antigen (HBs) | Hepatitis B |
| H and F proteins | Measles |
| G and F proteins | Nipah virus |
| VP4 and VP8 | Rotavirus |
| G and F proteins | Human Metapneumo virus |
| HN and F proteins | Parainfluenza virus |
| Sika envelope domain | Zika |
| III (ZEDIII) | |

In some embodiments, the antigen is a bacterial antigen. For example, the antigen may be derived from an infectious bacteria including, but not limited to, Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae (Group B Streptococcus), Streptococcus (viridans group), Streptococcus faecalis, streptococcus bovis, Streptococcus (anaerobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii. Suitable bacterial antigens are identified in Table 3 below.

TABLE 3
Exemplary Bacterial Antigens

| Antigen | Bacteria |
|---|---|
| Pfs25 protein (Pfs25) | Plasmodium falciparum |
| Major outer membrane protein (MOMP) | Chlamydia |
| SchuS4 O-antigen polysaccharide | Francisella tularensis |
| (FtO-PS) | |
| Outer membrane protein | Haemophilus influenzae type b |
| M protein | Streptococcus pyogenes |
| Toxoid A, toxoid B | Clostridium difficile |

In some embodiments, the bacterial antigen is selected from the group consisting of Chlamydia major outer membrane protein (MOMP) and Francisella tularensis SchuS4 O-antigen polysaccharide (FtO-PS), or a fragment thereof.

In some embodiments, the antigen is a parasitic antigen. For example, the antigen may be derived from the group consisting of blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

TABLE 4
Exemplary Parasitic Antigens

| Antigen | Parasite |
|---|---|
| circumsporozoite (PfCSP), sporozoite | Plasmodium falciparum |
| surface protein 2 (PfSSP2), exported | |
| protein 1 (PfExp-1), Pfs25 protein (Pfs25) | |
| ASP-1 and ASP-2 | Ancylostoma caninum |

In some embodiments, the parasitic antigen is Plasmodium falciparum Pfs25 protein (Pfs25).

In some embodiments, the antigen is a fungal antigen. For example, the antigen may be derived from the group consisting of Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

In some embodiments, the antigen is an allergen. Suitable exemplary allergens may be derived from the following genera: Canine, Dermatophagoides, Felis, Ambrosia, Lotium, Cryptomeria, Alternaria, Alder, Alinus, Betula, Quercus, Olea, Artemisia, Plantago, Parietaria, Blatella, Apis, Cupressus, Juniperus, Thuya, Chamaecyparis, Periplanet, Agopyron, Secale, Triticum, Dactylis, Festuca, Poa, Avena, Holcus, Anthoxanthum, Arrhenatherum, Agrostis, Phleum, Phalaris, Paspalum, Sorghum, and Bromis.

In some embodiments, the therapeutic composition further comprises a pharmaceutically-acceptable carrier. As used herein, the term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives, or buffers, which enhance the shelf life or effectiveness of the therapeutic agent.

The therapeutic compositions according to the present disclosure may be prophylactic (i.e., to prevent infection) or therapeutic (i.e., to treat infection), but will typically be prophylactic. In accordance with such embodiments, the synthetic antigen receptor displays the biotinylated antigen on the surface of the outer membrane vesicle. Suitable antigens (e.g., human, viral, bacterial, parasitic, and fungal antigens) are described supra. In some embodiments, the antigen is a vaccine subunit antigen. Immunogenic compositions used as vaccines comprise an immunologically effective amount of antigen(s), as well as any other components, as needed. An immunologically effective amount, is the amount administrated to an individual, either in a single dose or as part of a series, that is effective for treatment or prevention. The amount varies depending upon the health and physical condition of the individual to be treated, age, the taxonomic group of individual to be treated (e.g., non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

Methods for preparing cellular vesicles suitable for administration as a therapeutic composition and methods and formulations for administration of cellular vesicles are known in the art and described herein and in WO2002/0028215 to Kadurugamuwa and Beveridge, WO2006/024946 to Oster et al., and WO2003/051379 to Foster et al., which are hereby incorporated by reference in their entirety.

The therapeutic compositions of the present disclosure can be formulated into pharmaceutically acceptable compositions for patient administration. An effective quantity of the outer membrane vesicles comprising the synthetic antigen receptor coupled to the biotinylated antigen according to the present disclosure are combined with a pharmaceutically acceptable vehicle as described, for example, in Remington's Pharmaceutical Sciences (Remington's Pharmaceutical Sciences, Mack Publishing Company, Easton, Pa., USA 1985, which is hereby incorporated by reference in its entirety). On this basis, the pharmaceutical compositions include, albeit not exclusively, solutions of the membrane vesicles in association with one or more pharmaceutically acceptable vehicles or diluents, and contained in buffered solutions with a suitable pH and isoosmotic with the physiological fluids.

The therapeutic compositions of the present disclosure can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The therapeutic compositions of the present disclosure may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, the therapeutic compositions may be incorporated with excipients and used in the form of tablets, capsules, elixirs, suspensions, syrups, and the like. Such compositions and preparations should contain at least 0.1% of outer membrane vesicle comprising the synthetic antigen receptor coupled with the biotinylated antigen according to the present disclosure. The percentage of the outer membrane vesicle comprising the synthetic antigen receptor coupled with the biotinylated antigen according to the present disclosure carrying the drug or vaccine in these compositions may, of course, be varied and may conveniently be between about 2% to about 60% of the weight of the unit. The amount of the biotinylated antigen in such therapeutically useful compositions is such that a suitable dosage will be obtained. Preferred compositions according to the present disclosure are prepared so that an oral dosage unit contains between about 1 and 250 mg of active drug or vaccine.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The therapeutic compositions according to the present disclosure may also be administered parenterally. Solutions or suspensions of these therapeutic compositions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The therapeutic compositions of the present disclosure may also be administered directly to the airways in the form of an aerosol. For use as aerosols, the therapeutic compositions of the present disclosure in solution or suspension may be packaged in a pressurized aerosol container together with suitable propellants, for example, hydrocarbon propellants like propane, butane, or isobutane with conventional adjuvants. The therapeutic compositions of the present disclosure also may be administered in a non-pressurized form such as in a nebulizer or atomizer.

The synthetic antigen receptor of the present disclosure may be encoded by a nucleic acid construct generated as described herein or using any other standard techniques known in the art. Accordingly, another aspect of the present disclosure relates to a nucleic acid construct encoding a system for displaying antigens comprising: a first nucleic acid sequence encoding a synthetic antigen receptor comprising at least a portion of an outer membrane scaffold protein; and a second nucleic acid sequence encoding a biotin-binding protein, where said first nucleic acid sequence is coupled to said second nucleic acid sequence.

Suitable outer membrane scaffold proteins (and portions of the outer membrane scaffold protein which is sufficient to be associated with an outer membrane vesicle) for use in the compositions and methods of the present disclosure are described in more detail supra. In some embodiments, the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

In some embodiments, the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

Suitable biotin-binding proteins for use in the compositions and methods of the present disclosure are described in more detail supra. In some embodiments, the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S2H}.

In some embodiments, the nucleic acid construct further includes a third nucleic acid sequence encoding a peptide linker, where the third nucleic acid sequence is positioned between said first nucleic acid sequence and said second nucleic acid sequence. Suitable peptide linkers for use in the compositions and methods of the present disclosure are described in more detail supra.

A nucleic acid construct encoding a SNARE according to the present disclosure may be inserted into an expression system to which the molecule is heterologous. The heterologous nucleic acid molecule is inserted into the expression system or vector in proper sense (5′ to 3′) orientation relative to the promoter and any other 5′ regulatory molecules, and correct reading frame. The preparation of the nucleic acid constructs can be carried out using standard cloning methods well known in the art as described by Sambrook et al., Molecular Cloning: A Laboratory Manual, Cold Springs Laboratory Press, Cold Springs Harbor, N.Y. (1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Accordingly, another aspect of the present disclosure relates to an expression vector for generating a system for displaying antigens comprising a nucleic acid construct according to the present disclosure.

Suitable expression vectors include those which contain replicon and control sequences that are derived from species compatible with the host cell. For example, if E. coli is used as a host cell, plasmids such as pUC19, pUC18, or pBR322 may be used.

Also contemplated are host cells comprising an expression vector according to the present disclosure. Host cells suitable for expressing and displaying the synthetic antigen receptors according to the present disclosure on an outer membrane vesicle surface include any one of the more commonly available gram negative bacteria. Suitable microorganisms include, without limitation, Pseudomonas aeruginosa, Escherichia coli, Salmonella gastroenteritis (typhimirium), S. typhi, S. enteriditis, Shigella flexneri, S. sonnie, S dysenteriae, Neisseria gonorrhoeae, N. meningitides, Haemophilus influenzae, H. pleuropneumoniae, Pasteurella haemolytica, P. multilocida, Legionella pneumophila, Treponema pallidum, T. denticola, T. orates, Borrelia burgdorferi, Borrelia spp., Leptospira interrogans, Klebsiella pneumoniae, Proteus vulgaris, P. morganii, P. mirabilis, Rickettsia prowazeki, R. typhi, R. richettsii, Porphyromonas (Bacteroides) gingivalis, Chlamydia psittaci, C. pneumoniae, C. trachomatis, Campylobacter jejuni, C. intermedis, C. fetus, Helicobacter pylori, Francisella tularenisis, Vibrio cholerae, Vibrio parahaemolyticus, Bordetella pertussis, Burkholderie pseudomallei, Brucella abortus, B. susi, B. melitensis, B. canis, Spirillum minus, Pseudomonas mallei, Aeromonas hydrophila, A. salmonicida, and Yersinia pestis. Methods for transforming/transfecting host cells with expression vectors are well-known in the art and depend on the host system selected as described in Sambrook et al., Molecular Cloning: A Laboratory Manual, Cold Springs Laboratory Press, Cold Springs Harbor, N.Y. (1989), which is hereby incorporated by reference in its entirety.

Following transformation of a host cell with an expression vector comprising a nucleic acid construct encoding the synthetic antigen receptor according to the present disclosure, the synthetic antigen receptor is expressed and displayed on the cell surface as well as the surface of outer membrane vesicles.

Another aspect of the present disclosure relates to a method of eliciting an immune response in a subject. This method involves administering a therapeutic composition comprising (i) an outer membrane vesicle comprising a lipid bilayer; (ii) a synthetic antigen receptor comprising at least a portion of an outer membrane scaffold protein fused to a biotin-binding protein, wherein the at least a portion of the outer membrane scaffold protein is incorporated in the lipid bilayer and the biotin-binding protein is displayed outside the outer membrane vesicle; and (iii) a biotinylated antigen bound to the biotin-binding protein, where the therapeutic composition is administered to the subject to elicit the immune response.

In accordance with this and all other aspects of the present invention, the term “immune response” refers to the development in a subject of a humoral and/or a cellular immune response to an antigen present in the composition of interest. A “humoral immune response” refers to an immune response mediated by antibody molecules, while a “cellular immune response” is one mediated by T-lymphocytes and/or other white blood cells. The antigen of interest may also elicit an antibody-mediated immune response. Hence, an immunological response may include one or more of the following effects: the production of antibodies by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The therapeutic compositions according to the present disclosure may be administered to the subject using methods known in the art including parenteral, topical, intravenous, oral, subcutaneous, intraperitoneal, intranasal or intramuscular means. The most typical route of administration for compositions formulated to induce an immune response is subcutaneous although others can be equally as effective. The next most common is intramuscular injection. This type of injection is most typically performed in the arm or leg muscles. Intravenous injections as well as intraperitoneal injections, intraarterial, intracranial, or intradermal injections are also effective in generating an immune response.

The therapeutic compositions of the present disclosure may be formulated for parenteral administration. Solutions or suspensions of the therapeutic composition can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the therapeutic compositions of the present disclosure systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present disclosure can also be achieved using infusion pump devices such as those described by Medtronic, Northridge, Calif. Such devices allow continuous infusion of desired compounds avoiding multiple injections and multiple manipulations.

In addition to the formulations described previously, the compositions of the present disclosure may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Detection of an effective immune response may be determined by a number of assays known in the art. For example, a cell-mediated immunological response can be detected using methods including, lymphoproliferation (lymphocyte activation) assays, CTL cytotoxic cell assays, or by assaying for T-lymphocytes specific for the antigen in a sensitized subject. Such assays are well known in the art.

The presence of a humoral immunological response can be determined and monitored by testing a biological sample (e.g., blood, plasma, serum, urine, saliva feces, CSF or lymph fluid) from the mammal for the presence of antibodies directed to the immunogenic component of the administered polymerized product. Methods for detecting antibodies in a biological sample are well known in the art, e.g., ELIS A, Dot blots, SDS-PAGE gels or ELISPOT. The presence of a cell-mediated immunological response can be determined by proliferation assays (CD4⁺ T cells) or CTL (cytotoxic T lymphocyte) assays which are readily known in the art.

Effective doses of the probiotic cells of the present invention, for the induction of an immune response, vary depending upon many different factors, including means of administration, target site, physiological state of the subject, whether the subject is a human or an animal, other medications administered, and whether treatment is prophylactic or therapeutic. Treatment dosages need to be titrated to optimize safety and efficacy, and could involve oral treatment.

In some embodiments of the methods disclosed herein, the vaccine composition further comprises a pharmaceutically-acceptable carrier. Suitable pharmaceutically-acceptable carriers for use in the compositions and methods according to the present disclosure are described in detail supra.

Suitable outer membrane scaffold proteins (and portions of the outer membrane scaffold protein which is sufficient to be associated with an outer membrane vesicle) for use in the compositions and methods of the present disclosure are described in more detail supra. In some embodiments, the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

In some embodiments, the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

Suitable biotin-binding proteins for use in the compositions and methods of the present disclosure are described in more detail supra. In some embodiments, the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S2H}.

Suitable synthetic antigen receptors for use in the compositions and methods of the present disclosure are described in more detail supra. Thus, in some embodiments of the methods disclosed herein, the synthetic antigen receptor is selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}. In some embodiments, the synthetic antigen receptor is selected from the group consisting of Lpp-OmpA-eMA, Lpp-OmpA-RA, or Lpp-OmpA-mSA^{S25H}.

In some embodiments, the method involves administering a therapeutic composition further comprising a peptide linker connecting the at least a portion of the outer membrane scaffold protein and the biotin-binding protein. Suitable peptide linkers are described in more detail supra.

In some embodiments, the biotinylated antigen comprises a globular protein, a membrane protein, a glycan, a glycoconjugate, a hapten, a saccharide, a lipid, a peptide, a nucleic acid, or combinations thereof. Suitable biotinylated antigens are described in more detail supra. In some embodiments, the biotinylated antigen is a selected to elicit an immune response against a human antigen.

In some embodiments, the biotinylated antigen is selected to elicit an immune response against a viral antigen. Suitable viral antigens are described supra.

In some embodiments, the biotinylated antigen is a selected to elicit an immune response against bacterial antigen. Suitable bacterial antigens are described supra. In some embodiments, the bacterial antigen is selected from the group consisting of Chlamydia major outer membrane protein (MOMP) and Francisella tularensis SchuS4 O-antigen polysaccharide (FtO-PS), or a fragment thereof.

In some embodiments, the biotinylated antigen is a selected to elicit an immune response against parasitic antigen. Suitable parasitic antigens are described supra. In some embodiments, the parasitic antigen is Plasmodium falciparum Pfs25 protein (Pfs25).

The subject may be a mammal. In some embodiments, the subject is a human. In other embodiments, the subject is a non-human mammal, e.g., a non-human primate, a dog, a cat, a rodent (e.g., a mouse, rat, guinea pig), a horse, a cattle, a sheep, or a pig.

In some embodiments, the method further involves selecting a subject at risk of developing a disease, disorder, or infection. In accordance with such embodiments, the immune response is effective to prevent the disease, disorder, or infection in the subject.

In other embodiments, the method further involves selecting a subject having a disease, disorder, or infection. In accordance with such embodiments, the immune response is effective to treat the disease, disorder, or infection in the selected subject.

In some embodiments, the immune response is effective to treat or prevent a disease, disorder, or infection in the subject. For example, the immune response may be effective to treat or prevent an infection such as a viral infection, a bacterial infection, a parasitic infection, or a fungal infection. In some embodiments the immune response is effective to treat or prevent a neoplastic conditions such as cancer. In other embodiments, the immune response is effective to treat or prevent an autoimmune disorder or allergic response in a subject.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

### EXAMPLES

The following examples are provided to illustrate embodiments of the present technology but are by no means intended to limit its scope.

### Materials and Methods for Examples 1-5

Strains, Growth Media, and Plasmids

All OMVs in this study were isolated from the hypervesiculating E. coli strain KPM404 ΔnlpI (Watkins et al., “Safe Recombinant Outer Membrane Vesicles that Display M2e Elicit Heterologous Influenza Protection,” Mol. Ther. 25:989-1002 (2017), which is hereby incorporated by reference in its entirety), which contains several genetic modifications that render its LPS less reactogenic. E. coli strain BL21(DE3) (Novagen) was used to express GFP and rCm-MOMP. The SIMPLEx constructs Sx-Cm-MOMP and Sx-CtE-MOMP were produced in two different ways, cell-based expression with BL21(DE3) or cell-free expression using an E. coli-based translation kit (RTS 500 ProteoMaster E. coli HY kit, Biotechrabbit GmbH) as described previously (He et al., “Cell-Free Production of a Functional Oligomeric form of a Chlamydia Major Outer-Membrane Protein (MOMP) for Vaccine Development,” J. Biol. Chem. 292:15121-15132 (2017), which is hereby incorporated by reference in its entirety). Both methods yield comparable amounts of similar quality products as assessed by SDS-PAGE and immunoblot analysis. E. coli strain CLM24 was used to produce CRM197 conjugated with FtO-PS (Cuccui et al., “Exploitation of Bacterial N-Linked Glycosylation to Develop a Novel Recombinant Glycoconjugate Vaccine Against Francisella tularensis,” Open Biol. 3:130002 (2013), which is hereby incorporated by reference in its entirety) while strain JC8031 (Bernadac et al., “Escherichia coli Tol-Pal Mutants form Outer Membrane Vesicles,” J. Bacteriol. 180:4872-4878 (1998), which is hereby incorporated by reference in its entirety) was used to produce recombinant FtLPS. Recombinant GFP used for serum antibody titering was expressed and purified from Saccharomyces cerevisiae strain SEY6210.1 to avoid cross-reaction of serum antibodies with contaminating host proteins present in protein preparations derived from E. coli cultures. The C. muridarum strain Nigg II (ATCC VR-123) was used to produce nCm-MOMP and EBs utilized in serum antibody titering experiments.

For cloning and strain propagation, E. coli strains were grown on solid Luria-Bertani LB (10 g/L tryptone, 10 g/L NaCl, and 5 g/L yeast extract) supplemented with agar (LBA) and yeast strain SEY6210.1 was grown on synthetic defined media without uracil (SD-URA; MP Biomedicals) supplemented with agar. For OMV production, hypervesiculating E. coli were grown in terrific broth (TB) (12 g/L tryptone, 24 g/L yeast extract, 0.4% v/v glycerol, 0.17 M KH₂PO₄ and 0.72 M K₂HPO₄). For production of recombinant antigens using E. coli, cells were grown in LB media. SEY6210.1 was grown in SD-URA or yeast extract-peptone-dextrose (YPD) media (20 g/L peptone, 10 g/L yeast extract and 2% w/v glucose). C. muridarum cells were grown as described (Nigg, “An Unidentified Virus which Produces Pneumonia and Systemic Infection in Mice,” Science 95:49-50 (1942) and Pal et al., “Protection Against Infertility in a BALB/c Mouse Salpingitis Model by Intranasal Immunization with the Mouse Pneumonitis Biovar of Chlamydia Trachomatis,” Infect. Immun. 62:3354-3362 (1994), which are hereby incorporated by reference in their entirety).

All plasmids used in the present disclosure are described in Table 5.

TABLE 5
Plasmids

| Name | Description | Reference* |
|---|---|---|
| pET24a(+)- | E. coli expression plasmid derived | Lab stock |
| Cm | from pET-24a(+) but with Cm^{R} | |
| | resistance marker; Cm^{R} | |
| pET24-GFP | Encodes FACS-optimized | Present disclosure |
| | GFPmut2 variant with C- terminal | |
| | 6xHis tag in pET-24a(+)-Cm; Cm^{R} | |
| pET21d-Sx | Encodes SIMPLEX components | Vidakovics et al., “B Cell Activation by |
| | MBP at the N- terminus and | Outer Membrane Vesicles--A Novel |
| | ApoAI* at the C-terminus with | Virulence Mechanism,” PLoS Pathog. |
| | multicloning site for insertion of | 6: e1000724 (2010), which is hereby |
| | POIs between MBP and ApoAI*; | incorporated by reference in its entirety |
| | Amp^{R} | |
| pET21-Sx- | Encodes Cm-MOMP in pET21d- | Present disclosure |
| Cm- MOMP | Sx; Amp^{R} | |
| pCM189 | Yeast expression plasmid with | McBroom and Kuehn, “Release of |
| | tetracycline- regulated promoter; | Outer Membrane Vesicles by Gram- |
| | Amp^{R} | Negative Bacteria is a Novel Envelope |
| | | Stress Response,” Mol. Microbiol. |
| | | 63: 545-558 (2007), which is hereby |
| | | incorporated by reference in its entirety |
| pCM-GFP | Encodes S. cerevisae codon- | Present disclosure |
| | optimized GFP with a C-terminal | |
| | 6xHis tag in pCM189; Amp^{R} | |
| pTrc99S- | Encodes DsbA signal peptide fused | Biller et al., “Bacterial Vesicles in |
| ssDsbA- | in-frame with CRM197 followed by | Marine Ecosystems,” Science 343: 183- |
| CRM197^{4XDQNAT} | a 4x tandemly repeated DQNAT | 186 (2014), which is hereby |
| | (SEQ ID NO: 2) glycosylation tag in | incorporated by reference in its entirety |
| | pTrc99S; Amp^{R} | |
| pGAB2 | Encodes F. tularensis SchuS4 O-PS | Gnopo et al., “Designer Outer |
| | biosynthesis pathway; Tet^{R} | Membrane Vesicles as |
| | | Immunomodulatory Systems - |
| | | Reprogramming Bacteria for Vaccine |
| | | Delivery,” Adv. Drug Deliv. Rev. |
| | | 114: 132-142 (2017), which is hereby |
| | | incorporated by reference in its entirety |
| pMAF10- | Encodes Campylobacter jejuni PglB | Rosenthal et al., “Pathogen-Like |
| PglB | oligosaccharyltransferase in | Particles: Biomimetic Vaccine Carriers |
| | pMAF10; Tmp^{R} | Engineered at the Nanoscale,” Curr. |
| | | Opin. Biotechnol. 28: 51-58 (2014), |
| | | which is hereby incorporated by |
| | | reference in its entirety |
| pIVEX2.4d | Plasmid for E. coli cell-based and | Jahromi and Fuhrmann, “Bacterial |
| | cell-free expression with a strong T7 | Extracellular Vesicles: Understanding |
| | promoter; Amp^{R} | Biology Promotes Applications as |
| | | Nanopharmaceuticals,” Adv. Drug. |
| | | Deliv. Rev. 173: 125-140 (2021), which |
| | | is hereby incorporated by reference in |
| | | its entirety |
| pIVEX-Sx- | Encodes SIMPLEX fusion MBP- | Present disclosure |
| CtE- MOMP | CtE-MOMP-ApoAI* in | |
| | pIVEX2.4d; Amp^{R} | |
| pET45-rCm- | Encodes Cm-MOMP without its | Li et al., “Bacterial Outer Membrane |
| MOMP | native signal peptide in pET- | Vesicles as a Platform for Biomedical |
| | 45b(+); Amp^{R} | Applications: An Update,” J. Control. |
| | | Release 323: 253-268 (2020), which is |
| | | hereby incorporated by reference in its |
| | | entirety |
| pClyA-GFP | Encodes ClyA-GFPmut2 fusion in | Alaniz et al., “Membrane Vesicles are |
| | pBAD18-Cm; Cm^{R} | Immunogenic Facsimiles of |
| | | Salmonella typhimurium that Potently |
| | | Activate Dendritic Cells, Prime B and |
| | | T Cell Responses, and Stimulate |
| | | Protective Immunity in vivo,” J. |
| | | Immunol. 179: 7692-7701 (2007), |
| | | which is hereby incorporated by |
| | | reference in its entirety |
| pBAD24- | Encodes ClyA-c-Myc-eMA-FLAG | Present disclosure |
| ClyA- eMA | fusion in pBAD24; Amp^{R} | |
| pBAD24- | Encodes Lpp-OmpA-c-Myc-eMA- | Present disclosure |
| Lpp- OmpA- | FLAG fusion in pBAD24; Amp^{R} | |
| eMA | | |
| pBAD24- | Encodes Int-c-Myc-eMA-FLAG fusion in | Present disclosure |
| Intimin- | pBAD24; Amp^{R} | |
| eMA | | |
| pBAD24- | Encodes spPelB-FLAG-eMA-c- | Present disclosure |
| eMA-Hbpβ | Myc-HBPβ fusion in pBAD24; | |
| | Amp^{R} | |
| pBAD24- | Encodes spPelB-FLAG-eMA-c- | Present disclosure |
| cMA-Ag43β | Myc-Ag43β fusion in pBAD24; | |
| | Amp^{R} | |
| pBAD24- | Encodes spPelB-FLAG-eMA-c- | Present disclosure |
| eMA-IgAPβ | Myc-IgAPβ fusion in pBAD24; | |
| | Amp^{R} | |
| pBAD24- | Encodes spPelB-FLAG-eMA- | Present disclosure |
| eMA-AIDA- | AIDA-Iβ fusion in pBAD24; Amp^{R} | |
| Iβ | | |
| pTrham | E. coli expression vector containing | Amid Biosciences |
| | L-rhamnose inducible promoter | |
| | rhaBAD; Amp^{R} | |
| pTrham-Lpp- | Encodes Lpp-OmpA-c-Myc-eMA- | Present disclosure |
| OmpA-eMA | FLAG fusion in pTrham; Amp^{R} | |
| pTrham- | Encodes ssPelB-FLAG-eMA-c-Myc- | Present disclosure |
| eMA-IgAPβ | IgAPβ fusion in pTrham; Amp^{R} | |
| pTrham-Lpp- | Encodes Lpp-OmpA-c-Myc- | Present disclosure |
| OmpA- | mSA^{S25H}-FLAG fusion in pTrham; | |
| SA^{S25H} | Amp^{R} | |
| pTrham-Lpp- | Encodes Lpp-OmpA-myc-SA-FLAG | Present disclosure |
| OmpA-SA | fusion in pTrham; Amp^{R} | |
| pTrham-Lpp- | Encodes Lpp-OmpA-myc-RA- | Present disclosure |
| OmpA-RA | FLAG fusion in pTrham; Amp^{R} | |

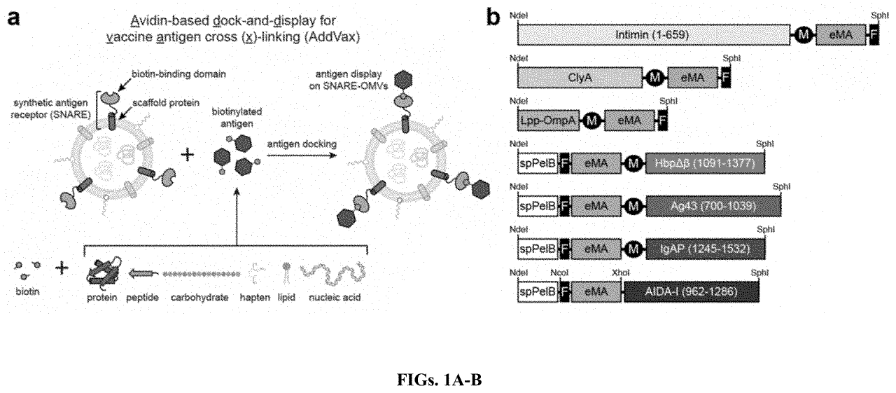

Standard restriction enzyme-based cloning methods were used and sequences were confirmed through Sanger sequencing performed by the Cornell Biotechnology Resource Center (BRC) unless specified otherwise. For expression of SNARE constructs in OMVs, eMA fusions to ClyA, Lpp-OmpA, and the membrane-associated transporter domains of the autotransporters Int, Hbp, Ag43, and IgAP were codon-optimized for E. coli expression, synthesized, and cloned into plasmid pBAD24 (Guzman et al., “Tight Regulation, Modulation, and High-Level Expression by Vectors Containing the Arabinose PBAD Promoter,” J. Bacteriol. 177:4121-4130 (1995), which is hereby incorporated by reference in its entirety) between EcoRI and SphI restriction sites with an NdeI site at the start codon by GenScript. SNARES involving ClyA, Lpp-OmpA and Int were cloned with eMA fused to the 3′ end of the scaffold while SNAREs involving Hbp, Ag43 and IgAP were cloned with eMA fused to the 5′ end of the scaffold (FIG. 1B). For the latter set of constructs, DNA encoding a Sec-dependent export signal peptide derived from PelB (spPelB), identical to the sequence in pET22b (Novagen), was introduced at the 5′-end of the eMA-scaffold gene fusions. For all of these constructs, DNA encoding c-Myc (EQKLISEEDL (SEQ ID NO:24)) and FLAG (DYKDDDDK(SEQ ID NO:1)) epitope tags was introduced at the 5′ and 3′ ends of eMA as depicted in FIG. 1B. In the case of the autotransporter AIDA-I, the transporter unit (amino acids 962 through 1286) was PCR-amplified from pIB264 (Benz et al., “AIDA-I, the Adhesin Involved in Diffuse Adherence of the Diarrhoeagenic Escherichia coli Strain 2787 (0126:H27), is Synthesized via a Precursor Molecule,” Mol. Microbiol. 6:1539-1546 (1992), which is hereby incorporated by reference in its entirety) and ligated into pBAD24 between XhoI and SphI restriction sites. A “gBlock” (Integrated DNA Technologies, IDT) encoding eMA with a 5′ FLAG tag (IDT) was ligated between NcoI and XhoI restriction sites, after which a gBlock encoding spPelB (IDT) was ligated between EcoRI and NcoI.

To construct L-rhamnose inducible plasmids, DNA encoding Lpp-OmpA-eMA and eMA-IgAP was digested from the respective pBAD24 expression vectors and ligated into pTrham (Amid Biosciences) between NdeI and SphI sites, yielding plasmids pTrham-Lpp-OmpA-eMA and pTrham-eMA-IgAP, respectively. To construct SNAREs based on alternative avidin domains, Rhizobium etli RA, Streptomyces avidinii SA, and an optimized version of monomeric streptavidin, namely mSA^{S25H}, with a lowered off-rate (Demonte et al., “Structure-Based Engineering of Streptavidin Monomer with a Reduced Biotin Dissociation Rate,” Proteins 81:1621-1633 (2013), which is hereby incorporated by reference in its entirety) were codon-optimized and synthesized as gBlocks with flanking BbsI and HindIII restriction sites (IDT). The sequences were then used to replace the eMA sequence in the pTrham-Lpp-OmpA-eMA vector, resulting in plasmids pTrham-Lpp-OmpA-RA, pTrham-Lpp-OmpA-SA, and pTrham-Lpp-OmpA-mSA.

For expression of GFP antigen for docking on OMVs, the gene encoding FACS-optimized GFPmut2 with a C-terminal 6xHis tag was cloned in pET24a(+)-Cm^{R} between SacI and HindIII restriction sites, yielding pET24-GFP. For yeast expression of GFP used in serum antibody titering, a codon-optimized gene encoding GFPmut2 was synthesized as a double-stranded DNA fragment or gBlock (IDT) with a 5′ Kozak sequence and 3′ 6xHis tag and ligated into the yeast-expression plasmid pCM189 (ATCC) between BamHI and PstI sites, yielding pCM-GFP. For expression of the Sx-Cm-MOMP antigen for OMV docking studies, the sequence encoding codon-optimized Cm-MOMP, which was designed previously (He et al., “Cell-Free Production of a Functional Oligomeric form of a Chlamydia Major Outer-Membrane Protein (MOMP) for Vaccine Development,” J. Biol. Chem. 292:15121-15132 (2017), which is hereby incorporated by reference in its entirety), was synthesized as a gBlock (IDT) and ligated into the SIMPLEx plasmid pET21d-Sx (Mizrachi et al., “A Water-Soluble DsbB Variant that Catalyzes Disulfide-Bond Formation in Vivo,” Nat. Chem. Biol. 13:1022-1028 (2017), which is hereby incorporated by reference in its entirety) between NdeI and EcoRI restriction sites, yielding pET21-Sx-Cm-MOMP. A modified strategy was used to generate plasmid pIVEX-Sx-CtE-MOMP encoding the Sx-CtE-MOMP construct. Briefly, codon optimized CtE-MOMP was generated in-house following a previously described strategy for Cm-MOMP (He et al., “Cell-Free Production of a Functional Oligomeric form of a Chlamydia Major Outer-Membrane Protein (MOMP) for Vaccine Development,” J Biol. Chem. 292:15121-15132 (2017), which is hereby incorporated by reference in its entirety). PCR products corresponding to CtE-MOMP, MBP, and ApoAI* (human ApoAI with 49 N-terminal amino acids removed) were cloned into pIVEX-2.4d using the following restriction enzyme strategy: NdeI-MBP-XhoI-MOMP-NsiI-ApoA1-SacI. The plasmid sequence was confirmed through Sanger sequencing performed by ElimBiopharm.

Protein Purification

For production of GFP and Sx-Cm-MOMP protein antigens, BL21(DE3) cells containing plasmids corresponding to each antigen were grown overnight at 37° C. in 5 mL LB supplemented with the appropriate antibiotic and subcultured 1:100 into the same media. Protein expression was induced with 0.1 mM isopropyl-β-D-1-thiogalactopyranoside (IPTG) when culture densities reached an absorbance at 600 nm (Abs₆₀₀) of ˜1.0 and proceeded for 16 hours at 30° C. Cells were then harvested and lysed by homogenization, and proteins were purified by Ni-NTA resin (Thermo-Fisher) following the manufacturer's protocol. For Sx-Cm-MOMP, Ni-NTA resin elute was immediately diluted with PBS containing 1 mM EDTA (PBS-E) and incubated with amylose resin (New England Biolabs) for 30 minutes, followed by washing with 10 resin volumes of PBS-E and elution with 10 mM maltose in PBS-E. All purified proteins were buffer exchanged into PBS using PD-10 desalting columns (Cytiva), filter-sterilized, quantified by Lowry (MilliporeSigma), and stored at 4° C. for up to 2 months or at −80° C. for longer term storage. Pfs25 was expressed and purified from a baculovirus expression system using Spodoptera frugiperda SF9 cells and P2 virus by Genscript.

To produce CRM197-FtO-PS glycoconjugate, E. coli strain CLM24 was transformed with plasmid pTrc99S-spDsbA-CRM197^{4×DQNAT} encoding the CRM197 carrier protein modified at its C-terminus with four tandemly repeated DQNAT (SEQ ID NO:2) glycosylation motifs (Stark et al., “On-Demand Biomanufacturing of Protective Conjugate Vaccines,” Sci. Adv. 7(6):eabe9444 (2021), which is hereby incorporated by reference in its entirety), plasmid pGAB2 encoding the FtO-PS biosynthesis pathway (Cuccui et al., “Exploitation of Bacterial N-Linked Glycosylation to Develop a Novel Recombinant Glycoconjugate Vaccine Against Francisella tularensis,” Open Biol. 3:130002 (2013), which is hereby incorporated by reference in its entirety), and plasmid pMAF10-Pg1B encoding the Campylobacter jejuni oligosaccharyltransferase Pg1B for transfer of the FtO-PS (Feldman et al., “Engineering N-Linked Protein Glycosylation with Diverse 0 Antigen Lipopolysaccharide Structures in Escherichia Coli,” Proc. Natl. Acad. Sci. USA 102:3016-3021 (2005), which is hereby incorporated by reference in its entirety). Overnight cultures were subcultured 1:100 into fresh LB containing appropriate antibiotics. When culture densities reached Abs₆₀₀ of ˜0.8, Pg1B expression was induced with 0.2% arabinose for 16 hours at 30° C., at which point CRM197^{4×DQNAT} expression was induced with 0.1 mM IPTG and cells were grown for an additional 8 hours at 30° C. Cells were then harvested and purified as described above for GFP.

To purify GFP for serum antibody titering, yeast strain SEY6210.1 was transformed with pCM189-GFP-6xHis and grown on SD-URA agar plates at 30° C. for two days. Afterwards, a colony was picked and grown overnight at 30° C. in 5 mL of SD-URA media containing tetracycline, subcultured 1:10 into YPD, and grown for 20 hours at 30° C. Yeast cells were lysed by homogenization and protein was purified by Ni-NTA resin as above. For Cm-MOMP serum antibody titering, rCm-MOMP was expressed recombinantly in E. coli while nCm-MOMP was extracted from C. muridarum strain Nigg II as described previously (Sun et al., “Protection Against an Intranasal Challenge by Vaccines Formulated with Native and Recombinant Preparations of the Chlamydia Trachomatis Major Outer Membrane Protein,” Vaccine 27:5020-5025 (2009), which is hereby incorporated by reference in its entirety).

Antigen Biotinylation

Purified GFP, Pfs25, Sx-Cm-MOMP, and CRM197-FtO-PS were mixed at 1 mg/mL (0.25-1 mg total protein) with 1.5× molar excess EZ-Link Sulfo-NHS-LC biotin (Thermo-Fisher) in PBS and incubated on ice for 2-3 hours. Afterwards, the reaction mix was passed five times over PBS-equilibrated monomeric avidin resin (Thermo-Fisher). Final flow-through fractions were concentrated and saved for repeat biotinylation reactions as needed. Following 6 washes each with one resin volume of PBS, biotinylated protein was eluted 6 times each with one resin volume of 2 mM D-biotin (MilliporeSigma) in PBS. Elutions were pooled and diluted to 6 mL with PBS and concentrated to <200 μL using 6-mL, 10-kDa cut-off protein concentrators (Pierce). Dilution and concentration was repeated three more times to remove the D-biotin. The final concentrated biotinylated proteins were filter-sterilized, quantified by Lowry, and stored at 4° C. for up to 2 months.

To biotinylate F. tularensis LPS, we first purified FtLPS as described previously (Chen et al., “Outer Membrane Vesicles Displaying Engineered Glycotopes Elicit Protective Antibodies,” Proc. Natl. Acad. Sci. USA 113:E3609-3618 (2016), which is hereby incorporated by reference in its entirety) with the addition of DNase-I (0.5 mg/mL; MilliporeSigma) in the Proteinase K treatment step. To remove sugar monomers and short polysaccharide chains, FtLPS was buffer exchanged into PBS using PD-10 columns and quantified by the Purpald assay (Leitner et al., “Lipopolysaccharide Modifications of a Cholera Vaccine Candidate Based on Outer Membrane Vesicles Reduce Endotoxicity and Reveal the Major Protective Antigen,” Infect. Immun. 81:2379-2393 (2013), which is hereby incorporated by reference in its entirety). Biotinylation was performed as described previously using 1-cyano-4-dimethylaminopyridinium tetrafluoroborate (CDAP) as the activation reagent linking EZ-Link-Amine-PEG3-Biotin (Pierce) to hydroxyl groups on the polysaccharide (Zhang et al., “Multiple Antigen-Presenting System (MAPS) to Induce Comprehensive B- and T-Cell Immunity,” Proc. Natl. Acad. Sci. USA 110:13564-13569 (2013), which is hereby incorporated by reference in its entirety). A 27-amino acid B16-M30 peptide with N-terminal biotin and C-terminal polyhistidine (6xHis) motif for antibody-based detection was synthesized by Biomatik to ˜85% purity, and a 1 mg/mL stock was prepared in dimethyl sulfoxide (DMSO). Biotinylated GD2 ganglioside oligosaccharide and biotinylated Le^{Y} oligosaccharide were purchased from Elicityl, biotinylated DNP containing a polyethylene glycol (PEG) linker was purchased from Nanocs, and 1-oleoyl-2-[12-biotinyl(aminododecanoyl)]-sn-glycero-3-phosphocholine (18:1-12:0 biotin PC) powder was purchased from Avanti Polar Lipids. The biotin-GD2, biotin-Le^{Y}, and biotin-DNP were dissolved in sterile water (1-5 mg/mL) while biotin-PC was suspended in DMSO (1 mg/mL). All stocks were diluted in PBS for avidin binding studies.

OMV Preparation

KPM404 ΔnlpI cells containing pBAD24 or pTrham expression plasmids were spread from −80° C. glycerol stocks onto LBA plates supplemented with 100 μg/ml carbenicillin and grown overnight at 37° C. (˜20 hours). On the following day, cells were suspended from the agar using TB and subcultured to Abs₆₀₀ of ˜0.06 in 50-100 mL TB supplemented with carbenicillin. Cells were grown at 37° C. and 220 rpm and induced when Abs₆₀₀ reached ˜0.6 to ˜1.8 with varying concentrations of L-arabinose (pBAD24) or L-rhamnose (pTrham). Following induction, cells were grown for 16 hours at 28° C. followed by 6 hours at 37° C., after which cells were pelleted via centrifugation at 10,000×g for 15 minutes. Supernatants were filtered through 0.2 μm filters and stored overnight at 4° C. OMVs were isolated by ultracentrifugation at 141,000×g for 3 hours at 4° C. and resuspended in sterile PBS. For quantitative analysis and immunizations, resuspended OMVs were diluted in sterile PBS and ultracentrifuged a second time to remove residual media and soluble proteins. Following a second resuspension in PBS, large irreversible aggregates were removed by centrifuging for 2 minutes at 3,000×g in a microcentrifuge and filtering the supernatant using sterile 4-mm, 0.45-μm syringe filters (MilliporeSigma). Total OMV proteins were quantified by Lowry (Peterson's modification; MilliporeSigma) using bovine serum albumin (BSA) as protein standard. OMVs were stored for up to 1 month at 4° C. for binding analysis and up to 2 weeks prior to immunizations.

Immunoblot Analysis

Biotinylated and non-biotinylated protein antigens and OMVs were mixed with loading buffer containing β-mercaptoethanol and boiled for 10 minutes prior to loading onto Mini-PROTEAN TGX polyacrylamide gels (Bio-Rad). To determine protein purity, gels were stained with Coomassie G-250 stain (Bio-Rad) following the manufacturer's protocol. For immunoblot analysis, proteins were transferred to polyvinylidene difluoride (PVDF) membranes and blocked with 5% milk followed by probing with antibodies, which were all used at 1:5,000 dilution. Avidin expression on OMVs was analyzed with horseradish peroxidase (HRP)-conjugated anti-c-Myc (Abcam; Cat #ab19312) or HRP-conjugated anti-DDDDK (SEQ ID NO:25)(Abcam; Cat #ab1162) antibodies that recognized c-Myc and FLAG epitope tags, respectively. Proteins and peptides bearing C-terminal 6xHis tags were detected with mouse anti-6xHis antibody clone AD1.1.10 (BioRad; Cat #MCA1396GA) while detection of glycosylated CRM197-FtO-PS was with anti-F. tularensis LPS antibody clone FB11 (Invitrogen; Cat #MA1-21690) that is specific to FtLPS (Chen et al., “Outer Membrane Vesicles Displaying Engineered Glycotopes Elicit Protective Antibodies,” Proc. Natl. Acad. Sci. USA 113:E3609-3618 (2016), which is hereby incorporated by reference in its entirety). HRP-conjugated goat anti-mouse (Abcam; Cat #ab6789) was used as needed. All membranes were developed using Clarity ECL substrate (Bio-Rad) and visualized using a ChemiDoc imaging system (Bio-Rad).

For probing antigenicity of SIMPLEx constructs, Sx-Cm-MOMP and Sx-CtE-MOMP were mixed with loading buffer containing DTT and boiled for 10 minutes before loading onto 4-12% NuPAGE Bis-Tris gels (Thermo-Fisher). For denaturing immunoblot analysis, proteins were transferred to PVDF membranes and blocked with 5% BSA (Sigma) followed by probing with mAb MoPn-40 (1:1,000) (Pal et al., “Protection of Wild-Type and Severe Combined Immunodeficiency Mice Against an Intranasal Challenge by Passive Immunization with Monoclonal Antibodies to the Chlamydia Trachomatis Mouse Pneumonitis Major Outer Membrane Protein,” Infect. Immun. 76:5581-5587 (2008), which is hereby incorporated by reference in its entirety) or anti-CtE-MOMP (1:2,000; Novus Biologicals; Cat #NB100-66403) antibodies. For non-denaturing dot blot analysis, purified Sx-Cm-MOMP and Sx-CtE-MOMP proteins were spotted directly onto nitrocellulose membranes and incubated for 5 minutes before being blocked with 5% BSA (Sigma) and probing with the same primary antibodies. IRDye 800CW-conjugated goat anti-mouse secondary antibodies (1:10,000; Li-Cor; Cat #926-32210) were used to detect primary antibody binding and membranes were visualized using an Odyssey Li-Cor Fc imaging system.

TEM Analysis of OMVs

Ultrastructural analysis of OMVs was performed via TEM as previously described (Chen et al., “Delivery of Foreign Antigens by Engineered Outer Membrane Vesicle Vaccines,” Proc. Natl. Acad. Sci. USA 107:3099-3104 (2010), which is hereby incorporated by reference in its entirety) with a few modifications. Briefly, OMVs were diluted to 100 μg/mL and negatively stained with 1.5% uranyl acetate and deposited on 300-mesh Formvar carbon-coated copper grids. Imaging was performed using a FEI Tecnai 12 BioTwin transmission electron microscope.

ELISA

For qualitative assessment of antigen binding by SNARE-OMVs, OMV samples were diluted to 2 μg/mL in PBS, coated on Costar 9018 high-binding 96-well plates (50 μL per well), and incubated overnight at 4° C. Plates were blocked with 2% BSA in PBS (100 μL/well) for 3 hours at room temperature and subsequently washed two times with PBST (PBS pH 7.4 with 0.005% Tween-20 and 0.3% BSA). To analyze relative binding capacities, biotinylated or unbiotinylated antigens were serially diluted in triplicate by a factor of 3 in PBST, starting from 10 nM, and incubated for 90 minutes at room temperature (50 μL/well). Unbound antigen was removed by washing twice with PBST. Bound antigen was labeled by incubating with primary antibody for 1 hour in PBST followed by two more PBST washes and a 1-hour incubation with HRP-conjugated secondary antibody. After three final washes with PBST, 3,3′-5,5′-tetramethylbenzidine substrate (1-Step Ultra TMB-ELISA; Thermo-Fisher) was added and the plate was incubated at room temperature for 30 minutes in the dark. The reaction was stopped with 2M H₂SO₄ and absorbance was measured via microplate spectrophotometer (Molecular Devices) at Abs₄₅₀. The absorbance reading from OMVs incubated with PBST without antigen was subtracted from the signal in all wells with antigen added. The resulting values were normalized to the highest average absorbance value among all antigen concentrations, including unbiotinylated antigen controls. Primary anti-6xHis and anti-FtLPS antibodies and HRP-conjugated anti-mouse secondary antibody were identical to those used for immunoblotting above and were used at the same dilutions. The remaining antigens were detected with the following antibodies: GD2 was detected with mouse anti-ganglioside GD2 antibody (1:1,000; Abcam; Cat #ab68456); Le^{Y} was detected with mouse anti-Lewis Y antibody clone H18A (1:1,000; Absolute Antibody; Cat #Ab00493-1.1); DNP was detected with goat anti-DNP (1:5,000; Bethyl Laboratories; Cat #A150-117A); and PC was detected with anti-phosphorylcholine antibody clone BH8 (1:250; MilliporeSigma; Cat #MABF2084). HRP-conjugated donkey anti-goat secondary was used as needed (1:5,000; Abcam; Cat #ab97110).

For quantification of antigen binding capacity on SNARE-OMVs, 50 μg OMVs were diluted to 0.1 mg/mL in PBS, mixed with unbiotinylated or biotinylated GFP at concentrations between 0 and 100 pmol/mL (0 to 1 pmol antigen/μg OMV), and incubated at room temperature for 1 hour. Mixtures were then diluted to 30 mL in PBS and ultracentrifuged for 141,000×g for 3 hours at 4° C. After discarding the supernatant, the pellet was resuspended with 100 μL PBS, and the washed OMVs were quantified by Lowry (Peterson's modification). An ELISA-based method that could be applied to a variety of molecules was then used to quantify the amount of antigen remaining. Specifically, washed OMVs were diluted to 2 μg/mL and coated on high-binding ELISA plates (Costar 9018) in triplicate (50 μL per well). Known standards were prepared by mixing blank SNARE-OMVs at 2 μg/mL with 1:2 serial dilutions of unbiotinylated or biotinylated GFP, starting from 1 pmol GFP/μg OMV, and coating each antigen concentration in triplicate on the ELISA plates (50 μL per well). Following overnight coating at 4° C., plates were blocked with 2% BSA in PBS for 3 hours (100 μL per well) at room temperature and subsequently washed two times with PBST. Antibody and substrate incubations were identical to the qualitative binding ELISA described above. The final Abs₄₅₀ signals of the OMV mixtures containing known amounts of unbiotinylated or biotinylated antigen were used to generate standard curves from which the amount of antigen remaining in each unknown washed OMV sample was calculated. The amount of GFP displayed on positive-control ClyA-GFP OMVs was quantified by fluorescence as described previously (Chen et al., “Delivery of Foreign Antigens by Engineered Outer Membrane Vesicle Vaccines,” Proc. Natl. Acad. Sci. USA 107:3099-3104 (2010), which is hereby incorporated by reference in its entirety).

Mouse Immunizations

One day prior to immunization (day −1), different OMV formulations were diluted to 0.1 mg/mL in sterile PBS pH 7.4. For the formulations involving docked antigens, antigens and OMVs were mixed to a final concentration of 100 pmol/mL and 0.1 mg/mL, respectively, corresponding to 1 pmol antigen/μg OMV (˜3 wt % for GFP). All formulations were immediately stored at 4° C. On day 0, 200 μL (20 μg) OMVs were injected subcutaneously into six-week-old BALB/c mice (7 mice per group). Identical booster injections were administered on days 21 and 42, and blood was drawn from the mandibular sinus on days −1, 28, and 49. Mice were euthanized on day 56, which was immediately followed by blood collection via cardiac puncture and spleen collection. The protocol number for the animal trial was 2012-0132 and was approved by the Institutional Animal Care and Use Committee (IACUC) at Cornell University.

Serum Antibody Titers

Sera was isolated from the blood of immunized mice by centrifugation at 5,000×g for 10 minutes and stored at −20° C. Antigen-specific antibodies in the sera were measured using indirect ELISA as described previously (Chen et al., “Outer Membrane Vesicles Displaying Engineered Glycotopes Elicit Protective Antibodies,” Proc. Natl. Acad. Sci. USA 113:E3609-3618 (2016)), which is hereby incorporated by reference in its entirety) with a few modifications. Briefly, high-binding 96-well plates (Costar 9018) were coated with purified antigen (5 μg/mL in PBS, pH 7.4) and incubated overnight at 4° C., followed by overnight blocking with 5% non-fat dry milk (Carnation) in PBS. Serum samples were serially diluted in triplicate by a factor of two in blocking buffer, starting from 1:100, and incubated on the antigen-coated plates for 2 hours at 37° C. Plates were washed 3 times with PBST and incubated for 1 hour at 37° C. in the presence of one of the following HRP-conjugated antibodies: goat anti-mouse IgG (1:10,000; Abcam Cat #ab6789); anti-mouse IgG1 (1:10,000; Abcam Cat #ab97240), or anti-mouse IgG2a (1:10,000; Abcam Cat #ab97245). Following 3 final washes with PBST, 1-Step Ultra TMB-ELISA (Thermo-Fisher) was added and the plate was incubated at room temperature for 30 minutes in the dark. The reaction was stopped with 2M H₂SO₄, and absorbance was quantified via microplate spectrophotometer (Molecular Devices) at Abs₄₅₀. Serum antibody titers were determined by measuring the highest dilution that resulted in signal three standard deviations above no-serum background controls.

The Chlamydia-specific antibody titers in sera from mice immunized with Sx-Cm-MOMP were determined by ELISA as previously described (Sun et al., “Protection Against an Intranasal Challenge by Vaccines Formulated with Native and Recombinant Preparations of the Chlamydia Trachomatis Major Outer Membrane Protein,” Vaccine 27:5020-5025 (2009), which is hereby incorporated by reference in its entirety). Briefly, 96-well plates were coated with 2 μg/ml of rCm-MOMP or nCm-MOMP, or 100 μL/well of C. muridarum EBs containing 10 μg/mL of protein in PBS. Next, 100 μL of serum was added per well in serial dilutions. Following incubation at 37° C. for 1 hour, the plates were washed, and HRP-conjugated goat anti-mouse IgG (1:10,000; BD Biosciences Cat #554002) was added. The plates were incubated and washed, and the binding was measured in an ELISA reader (Labsystem Multiscan) using 2,2′-azino-bis-(3-ethylbenzthiazoline-6-sulfonate) as the substrate.

Statistical Analysis

Statistical significance between groups was determined by unpaired t-test with Welch's correction using GraphPad Prism software (version 9.0.2). Statistical parameters including the definitions and values of n, p values, and SDs are reported in the figures and corresponding figure legends.

### Example 1—A Modular Framework for Self-Assembly of Antigens on OMV Surfaces

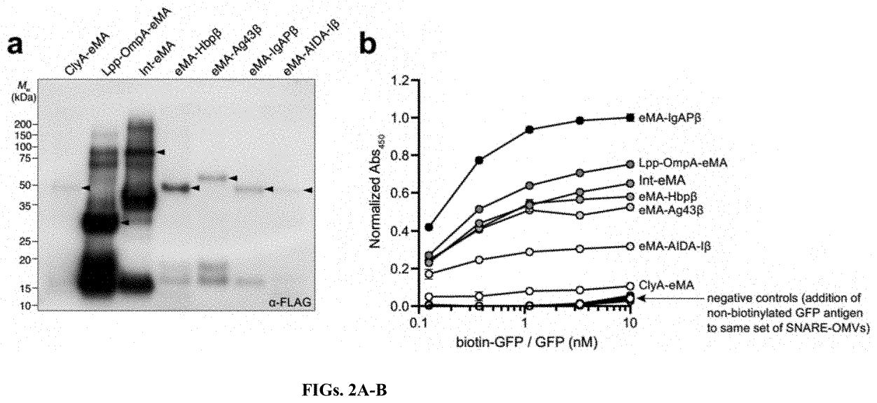

As a first step towards developing a universal platform for rapidly assembling antigens of interest on the surface of OMVs, SNAREs were constructed by translationally fusing a cell surface scaffold protein to a biotin-binding protein (FIG. 1A). A panel of cell surface scaffold modules were chosen based on their ability to direct passenger proteins to the E. coli outer membrane. These included cytolysin ClyA (Kim et al., “Engineered Bacterial Outer Membrane Vesicles with Enhanced Functionality,” J. Mol. Biol. 380:51-66 (2008), which is hereby incorporated by reference in its entirety), hybrid protein Lpp-OmpA (Francisco et al., “Transport and Anchoring of Beta-Lactamase to the External Surface of Escherichia coli,” Proc. Natl. Acad. Sci. USA 89:2713-2717 (1992), which is hereby incorporated by reference in its entirety), and the autotransporter β-domains derived from the N-terminus of intimin (Int) (Jong et al., “Extracellular Production of Recombinant Proteins Using Bacterial Autotransporters,” Curr. Opin. Biotechnol. 21:646-652 (2010), which is hereby incorporated by reference in its entirety) and the C-termini of adhesin involved in diffuse adherence (AIDA-I), antigen-43 (Ag43), hemoglobin-binding protease (Hbp), and immunoglobulin A protease (IgAP) (Jong et al., “Comparing Autotransporter Beta-Domain Configurations for their Capacity to Secrete Heterologous Proteins to the Cell Surface,” PLoS ONE 13:e0191622 (2018), which is hereby incorporated by reference in its entirety). Initially, each scaffold was fused in-frame to enhanced monoavidin (eMA) (FIG. 1B), a derivative of dimeric rhizavidin (RA) that was designed to be monomeric with highly stable, biotin-binding properties (Lee et al., “A Rhizavidin Monomer with Nearly Multimeric Avidin-Like Binding Stability Against Biotin Conjugates,” Angew Chem. Int. Ed. Engl. 55:3393-3397 (2016), which is hereby incorporated by reference in its entirety), and subsequently expressed from the arabinose-inducible plasmid pBAD24 in hypervesiculating E. coli strain KPM404 ΔnlpI (Mamat et al., “Detoxifying Escherichia Coli for Endotoxin-Free Production of Recombinant Proteins,” Microb. Cell Fact 14:57 (2015), which is hereby incorporated by reference in its entirety). This strain is an endotoxin-free BL21(DE3) derivative (sold as ClearColi™ by Lucigen) that was previously engineered to vesiculate through knockout of the nlpI gene (Watkins et al., “Safe Recombinant Outer Membrane Vesicles that Display M2e Elicit Heterologous Influenza Protection,” Mol. Ther. 25:989-1002 (2017), which is hereby incorporated by reference in its entirety). Using this strain, OMVs were readily produced that contained full-length SNARE chimeras, with Lpp-OmpA-eMA and Int-eMA showing the strongest expression albeit with significant amounts of higher and lower molecular weight species that likely corresponded to aggregation and degradation products, respectively (FIG. 2A).

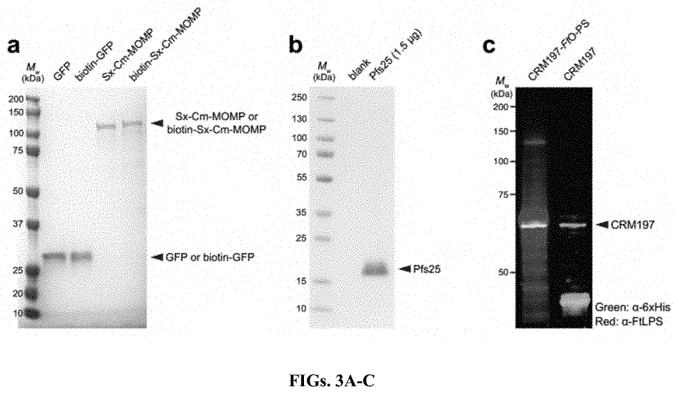

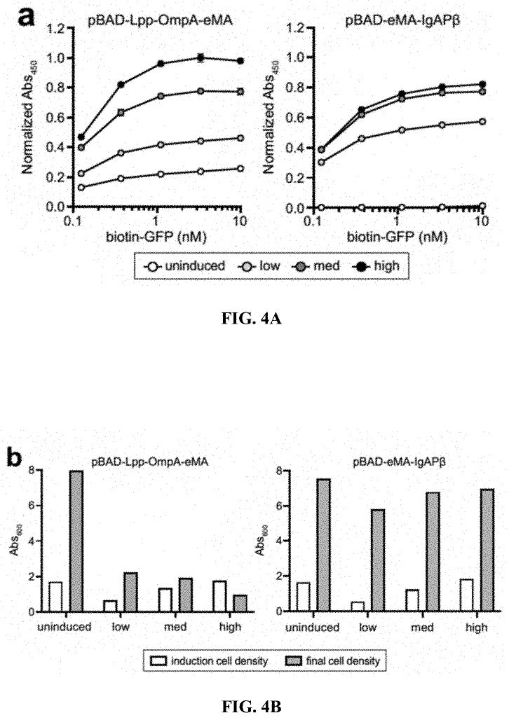

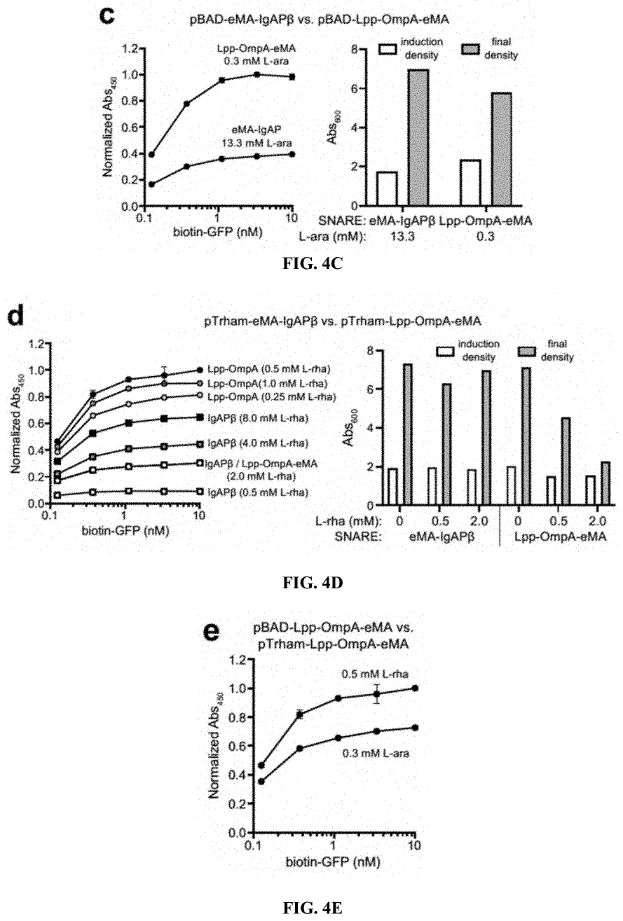

To evaluate antigen docking, the initial focus was on biotinylated green fluorescent protein (biotin-GFP) as the target antigen (FIG. 3A), which enabled facile and quantitative prototyping of the different SNARE-OMV designs. When biotin-GFP was incubated with 100 ng SNARE-OMVs coated on ELISA plates, all exhibited dose-dependent binding up to ˜10 nM of biotin-GFP except for the eMA-AIDA-Iβ and ClyA-eMA receptors, which appeared saturated at low levels of biotin-GFP (FIG. 2B). The lack of binding for these two SNAREs was not entirely surprising given that these constructs exhibited very weak expression compared to the other SNAREs (FIG. 2B). Importantly, there was no detectable binding of unmodified GFP by any of the SNARE-OMVs, indicating that antigen capture was entirely dependent upon the presence of the biotin moiety. Next, the two most effective SNAREs in terms of biotin-GFP binding, namely eMA-IgAPβ and Lpp-OmpA-eMA, were evaluated over a range of conditions to identify parameters (e.g., growth temperature, culture density at time of induction, inducer level, etc.) that affected GFP docking levels (discussed below, and shown in FIGS. 4A-4E).

A preliminary test of different cultivation variables (e.g., growth temperature, culture density at time of induction, inducer level, plasmid backbone, etc.), revealed that the density of the culture at the time of receptor induction had the greatest impact on the levels of biotin-GFP loading, with higher induction densities (Abs₆₀₀≈1.8) resulting in SNARE-OMVs that captured the most antigen (FIG. 3A). The Lpp-OmpA-eMA construct showed the highest biotin-GFP binding levels under the conditions tested. However, expression of this SNARE was detrimental to the host cells based on the observation that the final culture densities hardly changed, and in some cases even decreased, from the densities at the time of induction, which was not the case for IgAP-eMA (FIG. 3B). Given the different biogenesis pathways of the IgAP autotransporter versus the Lpp-OmpA β-barrel outer membrane protein, it is suspected that the host cell toxicity associated with Lpp-OmpA might result from inducer levels that were too strong. In support of this notion, when the Lpp-OmpA-eMA constructs were induced with ˜50-times less inducer (0.27 mM vs. 13.3 mM L-arabinose), the post-induction cell growth was markedly improved, with Lpp-OmpA-eMA-expressing cells reaching a final density on par with that of cells expressing IgAP-eMA (FIG. 3C). Importantly, the Lpp-OmpA-eMA SNARE-OMVs isolated from these healthier host cells captured significantly more biotin-GFP compared to IgAP-eMA SNARE-OMVs.

To determine whether these effects were specific to the choice of plasmid, an alternative plasmid was evaluated for expression of both SNAREs. Specifically, the IgAP-eMA and Lpp-OmpA-eMA constructs were re-cloned into the L-rhamnose-inducible plasmid, pTrham, which is known to afford tighter expression control compared to pBAD vectors and can help to overcome the deleterious saturation of membrane and secretory protein biogenesis pathways (Giacalone et al., “Toxic Protein Expression in Escherichia Coli Using a Rhamnose-Based Tightly Regulated and Tunable Promoter System,” Biotechniques 40:355-364 (2006); Hjelm et al., “Tailoring Escherichia Coli for the 1-Rhamnose PBAD Promoter-Based Production of Membrane and Secretory Proteins,” ACS Synth. Biol. 6:985-994 (2017), which are hereby incorporated by reference in their entirety). As was observed with pBAD24, cells expressing IgAP-eMA from pTrham reached similar final densities regardless of the inducer levels, while growth of cells expressing Lpp-OmpA-eMA from pTrham decreased with increasing inducer levels (FIG. 3D). Despite these differences in growth, IgAP-eMA and Lpp-OmpA-eMA SNARE-OMVs derived from cultures that were induced with 2 mM L-rhamnose each bound equivalent amounts of biotin-GFP (FIG. 3D). Interestingly, increasing the amount of L-rhamnose yielded IgAP-eMA SNARE-OMVs that captured 2-3 times more biotin-GFP whereas decreasing the amount of L-rhamnose yielded Lpp-OmpA SNARE-OMVs that bound 4-5 times more biotin-GFP, consistent with the contrasting effects of inducer on the post-induction growth of cells expressing these constructs. Overall, the engineered Lpp-OmpA-eMA receptor expressed from pTrham plasmid using 0.5 mM L-rhamnose was the strongest performer in terms of biotin-GFP binding (FIGS. 3D and 3E); hence, this plasmid/inducer combination was chosen for all further studies.

The engineered Lpp-OmpA-eMA receptor outperformed eMA-IgAPβ in terms of biotin-GFP binding capacity (FIG. 4A); however, expression of this construct from pBAD24 using standard amounts of L-arabinose (0.2% or 13.3 mM) was detrimental to the host cells based on the observation that the final culture densities hardly changed, and in some cases even decreased, from the densities at the time of induction, which was not the case for eMA-IgAPβ (FIG. 4B). Given the different biogenesis pathways of the IgAP autotransporter versus the Lpp-OmpA β-barrel outer membrane protein, it was suspected that the host cell toxicity associated with Lpp-OmpA might result from inducer levels that were too strong. In support of this notion, when Lpp-OmpA-eMA constructs were induced with approximately 50-times less inducer, the post-induction cell growth was markedly improved, with Lpp-OmpA-eMA-expressing cells reaching a final density on par with that of cells expressing eMA-IgAPβ (FIG. 4C). Importantly, the Lpp-OmpA-eMA SNARE-OMVs isolated from these healthier host cells captured significantly more biotin-GFP compared to eMA-IgAPβ SNARE-OMVs. An even higher level of biotin-GFP binding was obtained by moving the Lpp-OmpA-eMA construct into the L-rhamnose-inducible plasmid pTrham (FIGS. 4D-4F), which is known for its tighter expression control compared to pBAD vectors and can help to overcome the deleterious saturation of membrane and secretory protein biogenesis pathways (Giacalone et al., “Toxic Protein Expression in Escherichia Coli Using a Rhamnose-Based Tightly Regulated and Tunable Promoter System,” Biotechniques 40:355-364 (2006) and Hjelm et al., “Tailoring Escherichia Coli for the 1-Rhamnose PBAD Promoter-Based Production of Membrane and Secretory Proteins,” ACS Synth. Biol. 6:985-994 (2017), which are hereby incorporated by reference in their entirety).

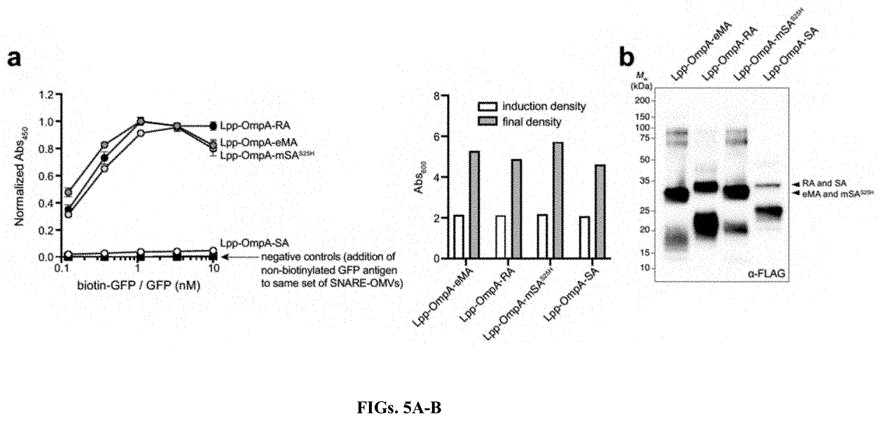

To determine the effect of the biotin-binding module on antigen loading and to further highlight the modularity of AddVax, a panel of Lpp-OmpA-based SNAREs comprised of alternative biotin-binding domains including dimeric RA, tetrameric streptavidin (SA), and monomeric streptavidin with a lowered off-rate (mSA^{S25H}) were constructed (Demonte et al., “Structure-Based Engineering of Streptavidin Monomer with a Reduced Biotin Dissociation Rate,” Proteins 81:1621-1633 (2013), which is hereby incorporated by reference in its entirety). The SNAREs comprised of RA and mSA^{S25H} both captured biotin-GFP at a level that was nearly identical to the eMA-based receptor, while the SA-based receptor showed binding that was barely above background, a result that appears to be due to the poor expression of this SNARE compared to the others (FIGS. 5A and 5B). Given the similarity in antigen capture efficiency for the eMA, RA, and mSA^{S25H} SNAREs as well as post-induction culture growth (FIG. 5A), the more extensively characterized Lpp-OmpA-eMA SNARE (expressed from plasmid pTrham with 0.5 mM L-rhamnose inducer) was chosen for all further studies.

### Example 2—Antigen Loading Capacity of SNARE-OMVs

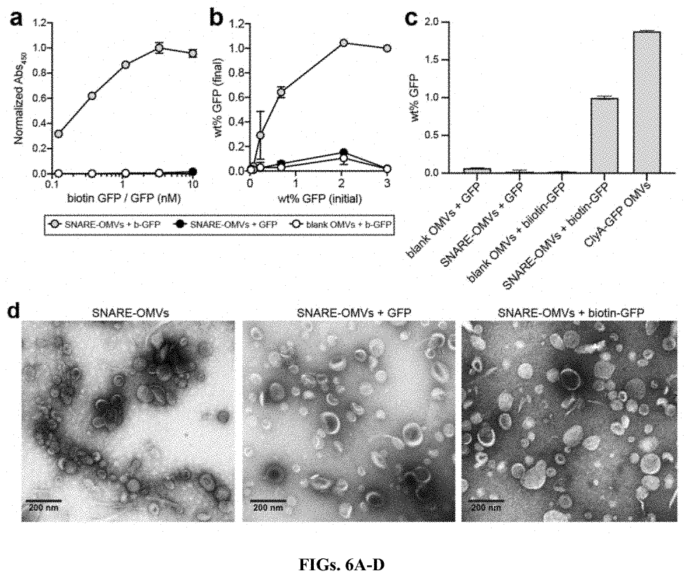

To determine the loading capacity of Lpp-OmpA-eMA SNARE-OMVs, the OMV fractions were first subjected to extensive washing with ultracentrifugation to recover washed OMVs, and then irreversible aggregates were removed by filtration through 0.45 μm pores. Next, bound antigen was quantified by mixing Lpp-OmpA-eMA SNARE-OMVs with biotin-GFP in solution and subsequently measuring the amount of OMV-bound GFP in an ELISA-style format. This assay was designed to mirror the process of vaccine self-assembly, whereby ready-made SNARE-OMVs are mixed with biotinylated antigens in an on-demand fashion. Importantly, the dose-response profile for pre-binding biotin-GFP on SNARE-OMVs in solution was in relative agreement with the dose-response curve generated by capturing biotin-GFP on the surface of immobilized SNARE-OMVs (FIG. 6A). The maximum amount of biotin-GFP that was captured on the SNARE-OMV surface was ˜1% by mass when ˜2 wt % biotin-GFP was input to the mixture, with the addition of higher amounts of biotin-GFP leading to no significant increase in biotin-GFP binding (FIG. 6B). In both assay formats, the combination of SNARE-OMVs with non-biotinylated GFP or biotin-GFP with blank OMVs lacking a SNARE resulted in little to no detectable binding (FIGS. 6A-6B). It was also found that the maximum biotin-GFP loading on SNARE-OMVs was lower but on par with the amount of GFP that was displayed on the surface of OMVs following cellular expression of a scaffold-antigen fusion, ClyA-GFP (FIG. 6C) (Kim et al., “Engineered Bacterial Outer Membrane Vesicles with Enhanced Functionality,” J. Mol. Biol. 380:51-66 (2008), which is hereby incorporated by reference in its entirety). Despite this difference, an advantage of SNARE-OMVs was the ability to vary the antigen loading density over a wide biotin-GFP concentration range, thereby providing a level of control that is more difficult to achieve with cellular expression of scaffold-antigen fusions. When visualized by transmission electron microscopy (TEM), SNARE-OMVs decorated with biotin-GFP had a size (˜50 nm) and overall appearance that was indistinguishable from unloaded SNARE-OMVs (FIG. 6D) and consistent with previous TEM images of engineered OMVs (Kim et al., “Engineered Bacterial Outer Membrane Vesicles with Enhanced Functionality,” J. Mol. Biol. 380:51-66 (2008); Chen et al., “Delivery of Foreign Antigens by Engineered Outer Membrane Vesicle Vaccines,” Proc Natl Acad Sci USA 107:3099-3104 (2010); and Rappazzo et al., “Recombinant M2e Outer Membrane Vesicle Vaccines Protect Against Lethal Influenza A Challenge in BALB/c Mice,” Vaccine 34:1252-1258 (2016), which are hereby incorporated by reference in their entirety) including those from the same hypervesiculating host strain used here (Giacalone et al., “Toxic Protein Expression in Escherichia Coli Using a Rhamnose-Based Tightly Regulated and Tunable Promoter System,” Biotechniques 40:355-364 (2006), which is hereby incorporated by reference in its entirety). These findings indicate that controllable vesicle loading could be achieved using the AddVax approach without significantly impacting OMV ultrastructure.

### Example 3—Decoration of OMV Surfaces with Structurally Diverse Antigens

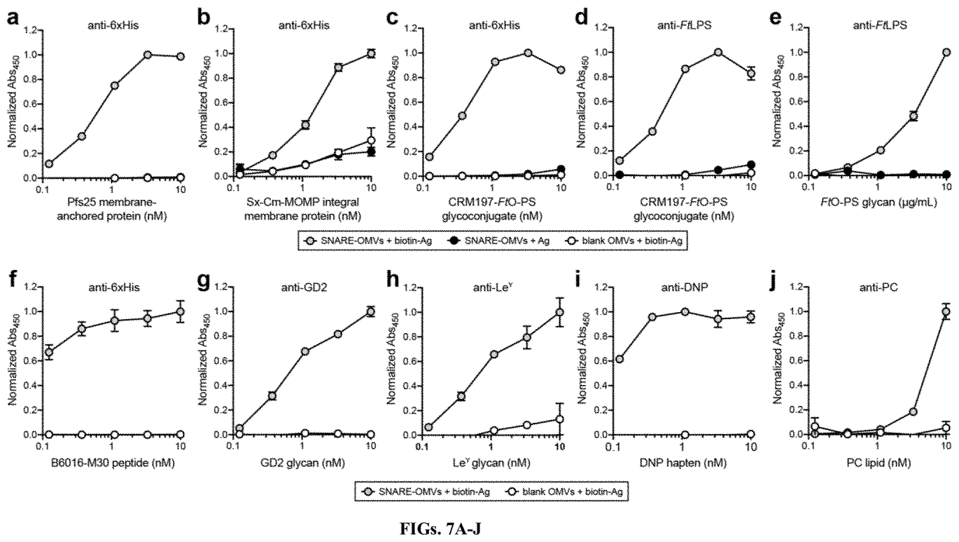

To demonstrate the universality of the AddVax system, decoration of SNARE-OMVs with a diverse array of biotinylated antigens was investigated next. Some of these were chosen because their incorporation into the OMV structure through cellular expression as a scaffold-antigen fusion protein was predicted to be difficult or impossible. For example, Plasmodium falciparum Pfs25 protein (Pfs25), a glycophosphotidylinositol (GPI)-anchored protein expressed on the surface of zygotes and ookinetes, is a promising malaria transmission-blocking vaccine antigen (Kaslow et al., “A Vaccine Candidate from the Sexual Stage of Human Malaria that Contains EGF-like Domains,” Nature 333:74-76 (1988), which is hereby incorporated by reference in its entirety). However, Pfs25 could not be expressed in soluble form in E. coli likely due to its 11 disulfide bonds (Lee et al., “Assessment of Pfs25 Expressed from Multiple Soluble Expression Platforms for use as Transmission-Blocking Vaccine Candidates,” Malar J 15:405 (2016), which is hereby incorporated by reference in its entirety), and thus is incompatible with conventional cellular expression techniques for OMV engineering. Along similar lines, Chlamydia major outer membrane protein (MOMP) is a β-barrel integral membrane protein (IMP) that accounts for approximately 60% of the mass of the outer membrane of Chlamydia spp. (Caldwell et al., “Purification and Partial Characterization of the Major Outer Membrane Protein of Chlamydia trachomatis,” Infect. Immun. 31:1161-1176 (1981) and Hatch et al., “Identification of a Major Envelope Protein in Chlamydia spp,” J. Bacteriol. 146:426-429 (1981), which are hereby incorporated by reference in their entirety) and is highly antigenic (Baehr et al., “Mapping Antigenic Domains Expressed by Chlamydia trachomatis Major Outer Membrane Protein Genes,” Proc. Natl. Acad. Sci. USA 85:4000-4004 (1988), which is hereby incorporated by reference in its entirety), making it an attractive subunit vaccine candidate (de la Maza et al, “Chlamydia trachomatis Vaccines for Genital Infections: Where Are We and How Far is There to Go?” Expert Rev Vaccines, 1-15 (2021), which is hereby incorporated by reference in its entirety). However, expression of MOMP in the E. coli cytoplasm results in aggregation and the formation of inclusion bodies (Hoelzle et al., “Expression of the Major Outer Membrane Protein (MOMP) of Chlamydophila abortus, Chlamydophila pecorum, and Chlamydia suis in Escherichia coli Using an Arabinose-Inducible Plasmid Vector,” J. Vet. Med. B. Infect. Dis. Vet. Public Health 50:383-389 (2003) and Sun et al., “Protection Against an Intranasal Challenge by Vaccines Formulated with Native and Recombinant Preparations of the Chlamydia trachomatis Major Outer Membrane Protein,” Vaccine 27:5020-5025 (2009), which are hereby incorporated by reference in their entirety) while expression in the E. coli outer membrane results in significant cell toxicity (Hoelzle et al., “Expression of the Major Outer Membrane Protein (MOMP) of Chlamydophila abortus, Chlamydophila pecorum, and Chlamydia suis in Escherichia coli Using an Arabinose-Inducible Plasmid Vector,” J. Vet. Med. B Infect. Dis. Vet. Public Health 50:383-389 (2003); Koehler et al., “Overexpression and Surface Localization of the Chlamydia trachomatis Major Outer Membrane Protein in Escherichia coli,” Mol. Microbiol. 6:1087-1094 (1992); and Wen et al., “Recombinant Expression of Chlamydia trachomatis Major Outer Membrane Protein in E. coli Outer Membrane as a Substrate for Vaccine Research,” BMC Microbiol. 16:165 (2016), which are hereby incorporated by reference in their entirety). To incorporate these two challenging membrane protein antigens into SNARE-OMVs required generation of soluble versions of each antigen. For Pfs25, soluble expression was achieved using a baculovirus-insect cell expression system (FIG. 3B), while for MOMP from Chlamydia trachomatis mouse pneumonitis (MoPn) biovar (strain Nigg II; now called Chlamydia muridarum), soluble expression was achieved using a protein engineering technology known as SIMPLEx (solubilization of IMPs with high levels of expression) (Mizrachi et al., “Making Water-Soluble Integral Membrane Proteins in Vivo Using an Amphipathic Protein Fusion Strategy,” Nat. Commun. 6:6826 (2015), which is hereby incorporated by reference in its entirety) in which sandwich fusion between an N-terminal “decoy” protein, namely E. coli maltose-binding protein (MBP), and C-terminal truncated human apolipoprotein AI (ApoAI*) transformed C. muridarum MOMP (Cm-MOMP) into a water-soluble protein that was expressed at high levels in the E. coli cytoplasm (FIG. 3A). Following incubation of SNARE-OMVs with biotinylated versions of insect cell-derived Pfs25 and E. coli-derived SIMPLEx-Cm-MOMP (Sx-Cm-MOMP), efficient OMV decoration that depended on both the presence of the chimeric Lpp-OmpA-eMA receptor on OMVs and the biotin moiety on each antigen was observed (FIG. 7A and FIG. 7B). In the case of Sx-Cm-MOMP, a low but reproducible signal for both controls was observed (SNARE-OMVs with non-biotinylated Sx-Cm-MOMP and blank OMVs with biotinylated Sx-Cm-MOMP) that may correspond to a small amount of auto-insertion of Sx-Cm-MOMP into OMVs.

Next, carbohydrate structures such as lipopolysaccharide (LPS) antigens that represent appealing molecules for vaccine development owing to their ubiquitous presence on the surface of diverse pathogens and malignant cells were investigated. A challenge faced with most polysaccharides is that they make poor vaccines on their own because they are unable to interact with the receptors on T cells in germinal centers (GCs) (Avci and Kasper, “How Bacterial Carbohydrates Influence the Adaptive Immune System,” Annu. Rev. Immunol. 28:107-130 (2010), which is hereby incorporated by reference in its entirety). This can be overcome by covalent attachment of a polysaccharide to a carrier protein, which provides T cell epitopes that can induce polysaccharide-specific IgM-to-IgG class switching, initiate the process of affinity maturation, and establish long-lived memory (Rappuoli, “Glycoconjugate Vaccines: Principles and Mechanisms,” Sci. Transl. Med. 10 (2018), which is hereby incorporated by reference in its entirety). Despite the widespread success of glycoconjugates, there is an unmet need to identify formulations that elicit stronger primary antibody responses after a single immunization, especially in primed or pre-exposed adolescents and adults, and achieve prolonged vaccine efficiency (Rappuoli, “Glycoconjugate Vaccines: Principles and Mechanisms,” Sci. Transl. Med. 10 (2018), which is hereby incorporated by reference in its entirety). To this end, it was speculated that AddVax would provide a convenient strategy for combining glycoconjugates with the intrinsic adjuvant properties of OMVs (Alaniz et al., “Membrane Vesicles are Immunogenic Facsimiles of Salmonella typhimurium that Potently Activate Dendritic Cells, Prime B and T Cell Responses, and Stimulate Protective Immunity in vivo,” J. Immunol. 179:7692-7701 (2007); Sanders and Feavers, “Adjuvant Properties of Meningococcal Outer Membrane Vesicles and the use of Adjuvants in Neisseria meningitidis Protein Vaccines,” Expert Rev Vaccines 10:323-334 (2011); and Ellis et al., “Naturally Produced Outer Membrane Vesicles from Pseudomonas aeruginosa Elicit a Potent Innate Immune Response via Combined Sensing of Both Lipopolysaccharide and Protein Components,” Infect. Immun. 78:3822-3831 (2010), which are hereby incorporated by reference in their entirety). Such an approach would provide a simpler alternative than attempting to combine OMV biogenesis with cellular expression of glycoconjugate vaccine candidates in E. coli (Kay et al., “Recent Advances in the Production of Recombinant Glycoconjugate Vaccines,” NPJ Vaccines 4:16 (2019), which is hereby incorporated by reference in its entirety), a feat that has yet to be reported. Thus, adorning SNARE-OMVs with biotinylated glycoconjugates was attempted by leveraging an engineered E. coli strain (Cuccui et al., “Exploitation of Bacterial N-Linked Glycosylation to Develop a Novel Recombinant Glycoconjugate Vaccine Against Francisella tularensis,” Open Biol 3:130002 (2013), which is hereby incorporated by reference in its entirety) to produce the carrier protein CRM197 that was glycosylated at its C-terminus with a recombinant mimic of the Francisella tularensis SchuS4 O-antigen polysaccharide (FtO-PS) (FIG. 3C). Decoration of SNARE-OMVs with a biotinylated version of this glycoconjugate was readily detected by immunoblotting against both the CRM197 carrier and its covalently linked FtO-PS antigen (FIGS. 7C and 7D, respectively).

An alternative strategy for combining OMVs with polysaccharide antigens whereby a biotinylated version of F. tularensis SchuS4 LPS (FtLPS) was directly bound to the exterior of SNARE-OMVs was also demonstrated (FIG. 7E). This formulation was motivated by the fact that a protein providing T cell help only needs to be in close proximity to the polysaccharide in order to target the same B cell and does not have to be covalently linked to the polysaccharide to induce class switching and T-cell activation (Chen et al., “Outer Membrane Vesicles Displaying Engineered Glycotopes Elicit Protective Antibodies,” Proc. Natl. Acad. Sci. USA 113:E3609-3618 (2016); Valentine et al., “Immunization with Outer Membrane Vesicles Displaying Designer Glycotopes Yields Class-Switched, Glycan-Specific Antibodies,” Cell Chem. Biol. 23:655-665 (2016); and Thanawastien et al., “Conjugate-Like Immunogens Produced as Protein Capsular Matrix Vaccines,” Proc. Natl. Acad. Sci. USA 112:E1143-1151 (2015), which are hereby incorporated by reference in their entirety). Indeed, the co-delivery of non-covalently linked proteins and polysaccharides present on the exterior of OMVs is sufficient to make a polysaccharide immunogenic (Vella et al., “Immunogenicity of a New Haemophilus influenzae Type B Conjugate Vaccine (Meningococcal Protein Conjugate) (PedvaxHIB),” Pediatrics 85:668-675 (1990); Chen et al., “Outer Membrane Vesicles Displaying Engineered Glycotopes Elicit Protective Antibodies,” Proc. Natl. Acad. Sci. USA 113:E3609-3618 (2016); and Valentine et al., “Immunization with Outer Membrane Vesicles Displaying Designer Glycotopes Yields Class-Switched, Glycan-Specific Antibodies,” Cell Chem. Biol. 23:655-665 (2016), which are hereby incorporated by reference in their entirety).

The final group of antigens that were investigated in this study were small-sized biomolecules that are known to be weakly immunogenic by themselves and therefore require carrier molecules to increase chemical stability and adjuvanticity for the induction of a robust immune response. This group included: (i) B16-M30 peptide, a CD4⁺ T-cell neoepitope expressed in the B16F10 melanoma as a consequence of a mutation in the kif18b gene (Kreiter et al., “Mutant MHC Class II Epitopes Drive Therapeutic Immune Responses to Cancer,” Nature 520:692-696 (2015), which is hereby incorporated by reference in its entirety); (ii) ganglioside GD2 glycan, a pentasaccharide antigen found on human tumors including melanoma, neuroblastoma, osteosarcoma, and small-cell lung cancer, that was highly ranked (12 out of 75) in a National Cancer Institute pilot program that prioritized the most important cancer antigens (Cheever et al., “The Prioritization of Cancer Antigens: A National Cancer Institute Pilot Project for the Acceleration of Translational Research,”. Clin. Cancer Res. 15:5323-5337 (2009), which is hereby incorporated by reference in its entirety); (iii) Lewis Y (Le^{Y}), a tetrasaccharide extension of the H blood group galactose-glucosamine that has been shown to be overexpressed on tumors (Kim et al., “Expression of LeY and Extended LeY Blood Group-Related Antigens in Human Malignant, Premalignant, and Nonmalignant Colonic Tissues,” Cancer Res. 46:5985-5992 (1986), which is hereby incorporated by reference in its entirety); (iv) 2,4-dinitrophenol (DNP), a model hapten to which the immune system is unresponsive (Feldman, “Induction of Immunity and Tolerance to the Dinitrophenyl Determinant in Vitro,” Nat. New Biol. 231:21-23 (1971), which is hereby incorporated by reference in its entirety); and (v) phosphocholine (PC), a major lipid component of myelin and one of the main antigenic targets of the autoimmune response in multiple sclerosis, with lipid-reactive antibodies likely contributing to disease pathogenesis (Sadaba et al., “Serum Antibodies to Phosphatidylcholine in MS,” Neurol. Neuroimmunol. Neuroinflamm. 7:(2020), which is hereby incorporated by reference in its entirety). In each case, detectable antigen binding on the surface of SNARE-OMVs that was significantly above the background seen with blank OMVs lacking biotin-binding receptors was clearly observed (FIGS. 7F-J). Collectively, these results illustrate the potential of the AddVax approach for modular self-assembly of candidate OMV vaccines decorated with diverse biomolecular cargo.

### Example 4—Immunogenicity of SNARE-OMVs Loaded with Model GFP Antigen

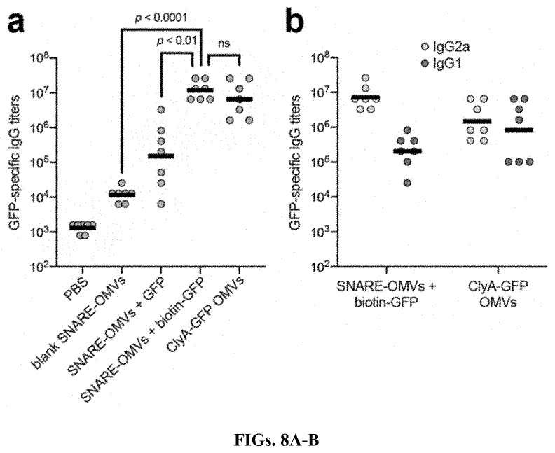

The immunological potential of SNARE-OMVs displaying biotin-GFP was assessed next. Specifically, BALB/c mice were immunized via subcutaneous (s.c.) injection of SNARE-OMVs decorated with biotin-GFP or other control formulations after which blood was collected at regular intervals. Negative controls included blank SNARE-OMVs, SNARE-OMVs that were mixed with non-biotinylated GFP, and PBS. ClyA-GFP-containing OMVs generated by cellular expression, which were previously reported to elicit high antibody titers following immunization in mice (Chen et al., “Delivery of Foreign Antigens by Engineered Outer Membrane Vesicle Vaccines,” Proc. Natl. Acad. Sci. USA 107:3099-3104 (2010), which is hereby incorporated by reference in its entirety), served as a positive control. Importantly, SNARE-OMVs displaying biotin-GFP elicited robust IgG responses to GFP that were significantly higher (p<0.0001) than the titers measured for control mice immunized with blank SNARE-OMVs or PBS (FIG. 8A). It is particularly noteworthy that the total IgG titers triggered by SNARE-OMVs were indistinguishable from those measured in response to ClyA-GFP-containing OMVs, validating the antigen docking strategy as a potent alternative to cellular expression of scaffold-antigen fusions for boosting the immunogenicity of foreign subunit antigens, in particular those that are weakly immunogenic on their own such as GFP (Chen et al., “Delivery of Foreign Antigens by Engineered Outer Membrane Vesicle Vaccines,” Proc. Natl. Acad. Sci. USA 107:3099-3104 (2010); Koser et al., “Rabies Virus Nucleoprotein as a Carrier for Foreign Antigens,” Proc. Natl. Acad. Sci. USA 101:9405-9410 (2004), which are hereby incorporated by reference in their entirety). Interestingly, the IgG response elicited by non-tethered GFP that was mixed with SNARE-OMVs gave a significantly lower (p<0.01) antigen-specific IgG response compared to biotin-GFP that was docked on OMVs, indicating that the physical coupling of the antigen to the surface of the OMV is essential for exploiting the full intrinsic adjuvanticity of OMVs. To determine whether the immune responses were Th1 or Th2 biased (Collins, “IgG Subclass Co-Expression Brings Harmony to the Quartet Model of Murine IgG Function,” Immunol. Cell Biol. 94:949-954 (2016), which is hereby incorporated by reference in its entirety), IgG antibody titers were further broken down by analyzing IgG1 and IgG2a subclasses. Mice immunized with different GFP-containing OMVs showed robust mean titers of both GFP-specific IgG1 and IgG2a antibodies (FIG. 8B). For the groups immunized with ClyA-GFP OMVs, the relative titers of IgG1 and IgG2a subclasses were comparable, consistent with earlier work and in line with responses typically seen with traditional subunit vaccines (Rosenthal et al., “Mechanistic Insight into the TH1-Biased Immune Response to Recombinant Subunit Vaccines Delivered by Probiotic Bacteria-Derived Outer Membrane Vesicles,” PLoS One 9:e112802 (2014), which is hereby incorporated by reference in its entirety). In contrast, biotin-GFP-studded SNARE-OMVs elicited an IgG2a-dominant humoral response, suggesting induction of a Th1-biased immune response consistent with heightened cellular immunity stimulation.

### Example 5—Immunogenicity of SNARE-OMVs Loaded with Validated Cm-MOMP Antigen

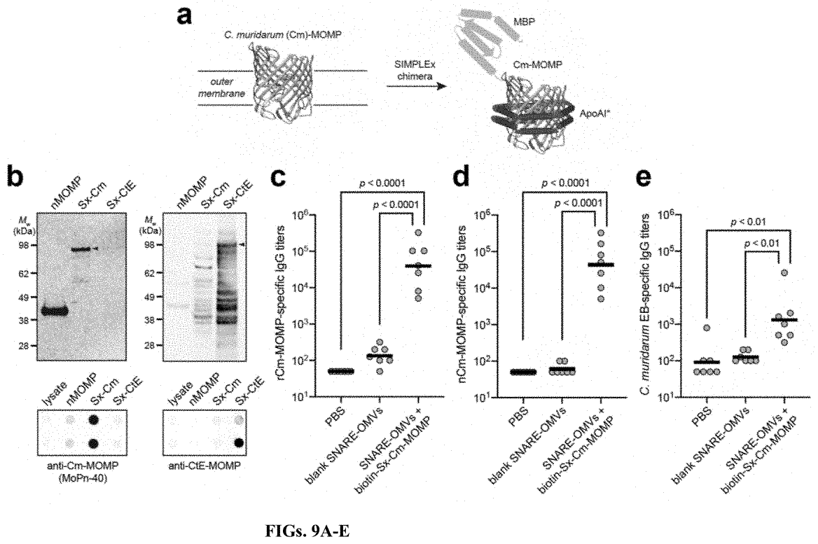

Encouraged by the immunostimulation observed for SNARE-OMVs remodeled with the model GFP antigen, the humoral immune response to SNARE-OMVs that were decorated with Cm-MOMP, a validated subunit vaccine candidate, were investigated next (de la Maza et al, “Chlamydia trachomatis Vaccines for Genital Infections: Where Are We and How Far is There to Go?” Expert Rev. Vaccines, 1-15 (2021) and Sun et al., “Protection Against an Intranasal Challenge by Vaccines Formulated with Native and Recombinant Preparations of the Chlamydia trachomatis Major Outer Membrane Protein,” Vaccine 27:5020-5025 (2009), which are hereby incorporated by reference in their entirety). Prior to immunization, the antigenicity of the Sx-Cm-MOMP construct (FIG. 9A) that was engineered as described above for soluble, high-level expression was first tested. Immunoblots of purified Sx-Cm-MOMP were probed with anti-Cm-MOMP-specific monoclonal antibody (mAb) MoPn-40, which was generated by inoculation of BALB/c mice with C. muridarum followed by isolation of hybridomas producing antibodies against Cm-MOMP (Pal et al., “Protection of Wild-Type and Severe Combined Immunodeficiency Mice Against an Intranasal Challenge by Passive Immunization with Monoclonal Antibodies to the Chlamydia trachomatis Mouse Pneumonitis Major Outer Membrane Protein,” Infect. Immun. 76:558-5587 (2008), which is hereby incorporated by reference in its entirety). It was observed that mAb MoPn-40 specifically recognized the water-soluble Sx-Cm-MOMP construct but not a SIMPLEx control construct comprised of a different MOMP from C. trachomatis serovar E (Sx-CtE-MOMP) in both denatured immunoblots and non-denatured dot blots (FIG. 9B), indicating that water-soluble Sx-Cm-MOMP retained conformational antigenicity.

Next, BALB/c mice were immunized s.c. with SNARE-OMVs decorated with biotinylated Sx-Cm-MOMP. When the resulting immune sera was analyzed for reactivity against either a recombinant or native preparation of Cm-MOMP (rCm-MOMP and nCm-MOMP, respectively) (Sun et al., “Protection Against an Intranasal Challenge by Vaccines Formulated with Native and Recombinant Preparations of the Chlamydia trachomatis Major Outer Membrane Protein,” Vaccine 27:5020-5025 (2009), which is hereby incorporated by reference in its entirety), strong cross-reaction to both antigens with total IgG titers that were significantly greater (p<0.0001) than the titers elicited by blank SNARE-OMVs and PBS control groups was observed (FIGS. 9C and 9D). It is also worth noting that the IgG responses triggered by Sx-Cm-MOMP docked on SNARE-OMVs were Chlamydia-specific as evidenced by the binding to C. muridarum elementary bodies (EBs), which was significantly above the binding measured for blank SNARE-OMVs and PBS control groups (FIG. 9E). As expected, antibody titers to rCm-MOMP and nCm-MOMP were similar while titers to EBs were lower. It should be pointed out that comparing titers between MOMP and EBs is not possible because the amount of MOMP present in EBs was not quantitated. Nonetheless, these data are significant because they demonstrate that the immune system of the mouse was able to recognize Cm-MOMP in the context of an OMV-tethered SIMPLEx construct. Taken together, these results confirm that dock-and-display of SIMPLEx-solubilized variants of membrane proteins on SNARE-OMVs is a unique approach for rapidly engineering vaccines based on difficult-to-obtain membrane-bound protein antigens without compromising antigenicity or immunogenicity.

### Discussion of Examples 1-5

In this study, a universal platform called AddVax for rapidly assembling antigens of interest on the surface of OMVs was developed. The method involves site-specific docking of biotinylated antigens to the exterior of ready-made OMVs displaying multiple copies of highly modular receptors called SNAREs, which are engineered by fusing an outer membrane scaffold domain to a biotin-binding domain. As shown herein, SNARE-OMVs can be readily adorned with virtually any antigen that is amenable to biotinylation including globular and membrane proteins, glycans and glycoconjugates, haptens, lipids, and short peptides. The ability to precisely and homogenously load OMVs with a molecularly diverse array of subunit antigens differentiates the AddVax method from previous covalent conjugation strategies that are largely restricted to protein and peptide antigens (Wu et al., “Sustained High-Titer Antibody Responses Induced by Conjugating a Malarial Vaccine Candidate to Outer-Membrane Protein Complex,” Proc. Natl. Acad. Sci. USA 103:18243-18248 (2006); Cheng et al., “Bioengineered Bacteria-Derived Outer Membrane Vesicles as a Versatile antigen Display Platform for Tumor Vaccination via Plug-and-Display Technology,” Nat. Commun. 12:2041 (2021); and van den Berg et al., “Display of Recombinant Proteins on Bacterial Outer Membrane Vesicles by Using Protein Ligation,” Appl. Environ. Microbiol. 84(8):e02567-17 (2018), which are hereby incorporated by reference in their entirety). Moreover, the dock-and-display approach described herein side-steps many of the challenges associated with display on OMVs using conventional genetic fusion and cellular expression technology, thereby opening the door to important vaccine subunit antigens such as malarial Pfs25 and Chlamydia Cm-MOMP that are refractory to soluble expression and outer membrane localization in E. coli (Lee et al., “Assessment of Pfs25 Expressed from Multiple Soluble Expression Platforms for use as Transmission-Blocking Vaccine Candidates,” Malar. J. 15:405 (2016); Hoelzle et al.,” Expression of the Major Outer Membrane Protein (MOMP) of Chlamydophila abortus, Chlamydophila pecorum, and Chlamydia suis in Escherichia coli Using an Arabinose-Inducible Plasmid Vector,” J. Vet. Med. B Infect. Dis. Vet. Public Health 50:383-389 (2003); Sun et al., “Protection Against an Intranasal Challenge by Vaccines Formulated with Native and Recombinant Preparations of the Chlamydia trachomatis Major Outer Membrane Protein,” Vaccine 27:5020-5025 (2009); Koehler et al., “Overexpression and Surface Localization of the Chlamydia trachomatis Major Outer Membrane Protein in Escherichia coli,” Mol Microbiol 6:1087-1094 (1992); and Wen et al., “Recombinant Expression of Chlamydia trachomatis Major Outer Membrane Protein in E. coli Outer Membrane as a Substrate for Vaccine Research,” BMC Microbiol 16:165 (2016), which are hereby incorporated by reference in their entirety). While the separate preparation of a biotinylated antigen adds an extra step, it affords an opportunity to generate protein antigens using different expression systems, which can be chosen based on their ability to promote high yields and desired conformations including post-translational modifications.

When injected in wild-type BALB/c mice, SNARE-OMV formulations displaying GFP or a water-soluble variant of Cm-MOMP were capable of triggering strong antigen-specific humoral responses that depended on the physical linkage between the antigen and the SNARE-OMV delivery vehicle. Importantly, the GFP-specific IgG titers elicited by GFP-studded SNARE-OMVs rivaled that of ClyA-GFP-containing OMVs generated by conventional cellular expression technology (Chen et al., “Delivery of Foreign Antigens by Engineered Outer Membrane Vesicle Vaccines,” Proc. Natl. Acad. Sci. USA 107:3099-3104 (2010), which is hereby incorporated by reference in its entirety). This ability of SNARE-OMVs to amplify the immunogenicity of GFP, a weakly immunogenic protein by itself (Chen et al., “Delivery of Foreign Antigens by Engineered Outer Membrane Vesicle Vaccines,” Proc. Natl. Acad. Sci. USA 107:3099-3104 (2010) and Koser et al., “Rabies Virus Nucleoprotein as a Carrier for Foreign Antigens,” Proc. Natl. Acad. Sci. USA 101:9405-9410 (2004), which are hereby incorporated by reference in their entirety), without the need for potentially reactogenic adjuvants indicates that the inbuilt adjuvanticity of OMVs is preserved in the context of our dock-and-display strategy. In the case of the validated subunit vaccine candidate, Cm-MOMP (de la Maza et al, “Chlamydia trachomatis Vaccines for Genital Infections: Where Are We and How Far is There to Go?” Expert Rev Vaccines, 1-15 (2021) and Sun et al., “Protection Against an Intranasal Challenge by Vaccines Formulated with Native and Recombinant Preparations of the Chlamydia trachomatis Major Outer Membrane Protein,” Vaccine 27:5020-5025 (2009), which are hereby incorporated by reference in their entirety), the potential of AddVax to be readily combined with SIMPLEx, a technology for solubilizing integral membrane proteins (Mizrachi et al., “Making Water-Soluble Integral Membrane Proteins in Vivo Using an Amphipathic Protein Fusion Strategy,” Nat. Commun. 6:6826 (2015) and Mizrachi et al., “A Water-Soluble DsbB Variant that Catalyzes Disulfide-Bond Formation in Vivo,” Nat. Chem. Biol. 13:1022-1028 (2017), which are hereby incorporated by reference in their entirety) was demonstrated, leading to an entirely new strategy for formulating difficult-to-obtain antigens without compromising immunogenicity.

Future adaptations of AddVax could also be pursued as needed, such as increasing antigen density with tandemly repeated biotin-binding modules or enabling multi-antigen display with SNAREs comprised of multiple orthogonal protein-ligand binding pairs. Along these lines, a trivalent protein scaffold containing three divergent cohesin domains for the position-specific docking of a three-enzyme cascade on the exterior of OMVs was previously engineered (Park et al., “Positional Assembly of Enzymes on Bacterial Outer Membrane Vesicles for Cascade Reactions,” PLoS One 9:e97103 (2014), which is hereby incorporated by reference in its entirety), which provides a conceptual starting point for next-generation SNARE-OMVs.

The AddVax technology is based on the extraordinarily high affinity of avidin for the small molecule biotin and was found to be compatible with a range of different biotin-binding modules including eMA, RA, and mSA^{S25H}. Although the binding affinity of the preferred biotin-binding domain, eMA, toward free biotin is measurably weaker than tetrameric SA (K_{d}=31×10⁻¹² M for eMA versus ˜10⁻¹⁴ M for SA), eMA is reported to have almost multimeric avidin-like binding stability toward biotin conjugates (Lee et al., “A Rhizavidin Monomer with Nearly Multimeric Avidin-Like Binding Stability Against Biotin Conjugates,” Angew Chem. Int. Ed. Engl. 55:3393-3397 (2016), which is hereby incorporated by reference in its entirety), making it an incredibly useful module for capturing diverse subunit antigens as shown here. Moreover, its small, monomeric design resulted in significantly better expression and OMV localization of the Lpp-OmpA-eMA SNARE compared to Lpp-OmpA-SA, which in turn resulted in far superior antigen capture. Another notable trait of eMA is its ability to be stored at −20° C. without visible aggregation or loss of binding function (Lee et al., “A Rhizavidin Monomer with Nearly Multimeric Avidin-Like Binding Stability Against Biotin Conjugates,” Angew Chem. Int. Ed. Engl. 55:3393-3397 (2016), which is hereby incorporated by reference in its entirety), which could prove useful in the future for long-term vaccine storage. It should be noted that the versatility of the avidin-biotin technology has been previously leveraged as a building material in other types of vaccine formulations, enabling the attachment of antigens onto virus-like particles (VLPs) (Chiba et al., “Multivalent Nanoparticle-Based Vaccines Protect Hamsters Against SARS-CoV-2 After a Single Immunization,” Commun. Biol. 4:597 (2021); Thrane et al., “A Novel Virus-Like Particle Based Vaccine Platform Displaying the Placental Malaria Antigen VAR2CSA,” PLoS ONE 10:e0143071 (2015); and Chackerian et al., “Conjugation of a Self-Antigen to Papillomavirus-Like Particles Allows for Efficient Induction of Protective Autoantibodies,” J. Clin. Invest. 108:415-423 (2001), which are hereby incorporated by reference in their entirety) and the self-assembly of macromolecular complexes comprised of vaccine antigens (Zhang et al., “Multiple Antigen-Presenting System (MAPS) to Induce Comprehensive B- and T-Cell Immunity,” Proc. Natl. Acad. Sci. USA 110:13564-13569 (2013); Leblanc et al., “VaxCelerate II: Rapid Development of a Self-Assembling Vaccine for Lassa Fever,” Human Vaccines & Immunotherapeutics 10:3022-3038 (2014), which are hereby incorporated by reference in their entirety). However, Applicant believe the study described herein is the first to repurpose avidin-biotin for antigen self-assembly and display on OMVs.

Overall, the AddVax platform enables creation of antigen-studded OMVs with the potential to impact many important facets of vaccine development. For example, the simplicity and modularity of vaccine self-assembly using AddVax enables rapid cycles of development and testing, which could be useful for evaluating large numbers and different combinations of pathogen-derived antigens for their ability to combat the most intractable diseases such as malaria or tuberculosis. Moreover, the fact that AddVax is based on an identical, easy-to-decorate SNARE-OMV scaffold that can be readily mass produced could shorten the time from development to manufacturing and accelerate regulatory review for each new vaccine candidate. The universal scaffold also affords the ability to share production costs across multiple antigens and diseases, which in combination with the favorable manufacturing economics of E. coli-based production, could help to meet the target of US $0.15 per human vaccine dose set by the Bill and Melinda Gates Foundation. In addition, pre-production of modular OMV scaffolds that can be stably stored at −20° C. and then only need to be mixed with good manufacturing practice (GMP)-grade biotinylated antigens could enable rapid responses to pathogen outbreaks or pandemics. One major remaining obstacle is the fact that OMVs derived from laboratory strains of E. coli have yet to enter the clinic. It should be noted, however, that OMVs/OMPCs from Neisseria meningitidis serogroup B are the basis of two licensed vaccines, PedvaxHIB® and Bexsero®, that are approved for use in humans (Vella et al., “Immunogenicity of a New Haemophilus influenzae Type B Conjugate Vaccine (Meningococcal Protein Conjugate) (PedvaxHIB),” Pediatrics 85:668-675 (1990) and Giuliani et al., “A Universal Vaccine for Serogroup B meningococcus,” Proc. Natl. Acad. Sci. USA 103:10834-10839 (2006), which are hereby incorporated by reference in their entirety). Hence, although more testing of SNARE-OMV vaccine candidates is clearly required, including broader immunogenicity testing and pathogen challenge studies, it is anticipated that clinical translation may not be far off.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A system for displaying antigens, said system comprising:
an outer membrane vesicle comprising a lipid bilayer and
a synthetic antigen receptor comprising an outer membrane scaffold protein fused to a biotin-binding protein, wherein the outer membrane scaffold protein is incorporated in the lipid bilayer and the biotin-binding protein is displayed outside the outer membrane vesicle.

2. The system according to claim 1, wherein the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

3. The system according to claim 1, wherein the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

4. The system according to claim 1, wherein the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}.

5. The system according to claim 1, wherein the synthetic antigen receptor is selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}.

6. The system according to claim 1 further comprising:
a peptide linker connecting the outer membrane scaffold protein and the biotin-binding protein.

7. A therapeutic composition comprising:
(i) an outer membrane vesicle comprising a lipid bilayer;
(ii) a synthetic antigen receptor comprising an outer membrane scaffold protein fused to a biotin-binding protein, wherein the outer membrane scaffold protein is incorporated in the lipid bilayer and the biotin-binding protein is displayed outside the outer membrane vesicle; and
(iii) a biotinylated antigen bound to the biotin-binding protein,
wherein the therapeutic composition is administered to the mammal under conditions effective to elicit an immune response.

8. The therapeutic composition according to claim 7 further comprising:
(iv) a pharmaceutically-acceptable carrier.

9. The therapeutic composition according to claim 7, wherein the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

10. The therapeutic composition according to claim 7, wherein the outer membrane scaffold protein is selected from the group consisting ofintimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

11. The therapeutic composition according to claim 7, wherein the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1,Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}.

12. The therapeutic composition according to claim 7, wherein the synthetic antigen receptor is selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}.

13. The therapeutic composition according to claim 7, wherein the synthetic antigen receptor is selected from the group consisting of Lpp-OmpA-eMA, Lpp-OmpA-RA, and Lpp-OmpA-mSA^{S25H}.

14. The therapeutic composition according to claim 7 further comprising:
a peptide linker connecting the outer membrane scaffold protein and the biotin binding protein.

15. The therapeutic composition according to claim 7, wherein the biotinylated antigen comprises a globular protein, a membrane protein, a glycan, a glycoconjugate, a hapten, a saccharide, a lipid, a peptide, a nucleic acid, or combinations thereof.

16. The therapeutic composition according to claim 15, wherein the biotinylated antigen is selected from the group consisting of a cancer antigen, a viral antigen, a parasitic antigen and a bacterial antigen.

17. The therapeutic composition according to claim 16, wherein the biotinylated antigen is a bacterial antigen selected from the group consisting of Chlamydia major outer membrane protein (MOMP) and Francisella tularensis SchuS4 O-antigen polysaccharide (FtO-PS).

18. The therapeutic composition according to claim 16, wherein the biotinylated antigen is a parasitic antigen and wherein the parasitic antigen is Plasmodium falciparum Pfs25 protein (Pfs25).

19. A nucleic acid construct encoding a system for displaying antigens comprising:
a first nucleic acid sequence encoding a synthetic antigen receptor comprising at least a portion of an outer membrane scaffold protein; and
a second nucleic acid sequence encoding a biotin-binding protein, wherein said first nucleic acid sequence is coupled to said second nucleic acid sequence.

20. The nucleic acid construct according to claim 19, wherein the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

21. The nucleic acid construct according to claim 19, wherein the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

22. The nucleic acid construct according to claim 19, wherein the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1, Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}.

23. The nucleic acid construct according to claim 19 further comprising:
a third nucleic acid sequence encoding a peptide linker, wherein said third nucleic acid sequence is positioned between said first nucleic acid sequence and said second nucleic acid sequence.

24. An expression vector for generating a system for displayhing antigens, the expression vector comprising:
the nucleic acid construct according to claim 19.

25. A method of eliciting an immune response in a subject, said method comprising:
administering a therapeutic composition comprising:
(i) an outer membrane vesicle comprising a lipid bilayer;
(ii) a synthetic antigen receptor comprising at least a portion of an outer membrane scaffold protein fused to a biotin-binding protein, wherein the at least a portion of the outer membrane scaffold protein is incorporated in the lipid bilayer and the biotin-binding protein is displayed outside the outer membrane vesicle; and
(iii) a biotinylated antigen bound to the biotin-binding protein, wherein the therapeutic composition is administered to the subject to elicit an immune response.

26. The method according to claim 25, wherein the therapeutic composition further comprises:
(iv) a pharmaceutically-acceptable carrier.

27. The method according to claim 25, wherein the outer membrane scaffold protein is selected from the group consisting of cytolysin (ClyA), Lpp-OmpA, the β domain of intimin (Int), β domain of hemoglobin-binding protease (Hbp), β domain of antigen-43 (Ag43), β domain of immunoglobulin A protease (IgAP), and the C-terminal domain of adhesin involved in diffuse adherence (AIDA-I).

28. The method according to claim 25, wherein the outer membrane scaffold protein is selected from the group consisting of intimin (1-659; SEQ ID NO:3), cytolysin (ClyA, SEQ ID NO:5), Lpp-OmpA (SEQ ID NO:7), HbpΔβ (1091-1377, SEQ ID NO:9), Ag43 (700-1039, SEQ ID NO:11), IgAP (1245-1532, SEQ ID NO:13), and AIDA-I (962-1286, SEQ ID NO:15).

29. The method according to claim 25, wherein the biotin-binding protein is selected from the group consisting of avidin, enhanced monoavidin (eMA), dimeric rhizavidin (RA), streptavidin (SA), Neutravidin, Bradavidin, Captavidin, Extravidin, NeutraLite, Tamavidin 1, Tamavidin 2, Avidin Related Proteins (AVR)1, AVR2, AVR3, AVR4, AVR5, AVR6, Bramavidin 1, Bramavidin 2, Burkavidin, Hoefavidin, Rhodavidin, Shwanavidin, Strongavidin, Xenavidin, Zebavidin, Beta6 avidins, Extended avidins, Metavidins, Legavidins, Animal avidins, Fungal avidins, Avidin-like proteins, Biotin-binding proteins, and monomeric streptavidin mSA^{S25H}.

30. The method according to claim 25, wherein the synthetic antigen receptor is selected from the group consisting of Int-eMA, ClyA-eMA, Lpp-OmpA-eMA, eMA-HbpΔβ, eMA-Ag43, eMA-IgAPβ, eMA-AIDA-Iβ, Lpp-OmpA-RA, and Lpp-OmpA-mSA^{S25H}.

31. The method according to claim 25, wherein the synthetic antigen receptor is selected from the group consisting of Lpp-OmpA-eMA, Lpp-OmpA-RA, or Lpp-OmpA-mSA^{S25H}.

32. The method according to claim 25, wherein the therapeutic composition further comprises:
a peptide linker connecting the at least a portion of the outer membrane scaffold protein and the biotin-binding protein.

33. The method according to claim 25, wherein the biotinylated antigen comprises a globular protein, a membrane protein, a glycan, a glycoconjugate, a hapten, a saccharide, a lipid, a peptide, a nucleic acid, or combinations thereof.

34. The method according to claim 25, wherein the biotinylated antigen is a selected to elicit an immune response against a human antigen.

35. The method according to claim 25, wherein the biotinylated antigen is selected to elicit an immune response against a viral antigen.

36. The method according to claim 25, wherein the biotinylated antigen is a selected to elicit an immune response against bacterial antigen.

37. The method according to claim 36, wherein the bacterial antigen is selected from the group consisting of Chlamydia major outer membrane protein (MOMP) and Francisella tularensis SchuS4 O-antigen polysaccharide (FtO-PS).

38. The method according to claim 25, wherein the biotinylated antigen is a parasitic antigen.

39. The method according to claim 38, wherein the parasitic antigen is Plasmodium falciparum Pfs25 protein (Pfs25).

40. The method according to claim 25, wherein the subject is a mammal.

41. The method according to claim 25, wherein the subject is a human.

42. The method according to claim 25, wherein said immune response is effective to treat a disease, disorder, or infection in the subject.

43. The method according to claim 25, wherein said immune response is effective to prevent a disease, disorder, or infection in the subject.
